(19) United States
(12) Patent Application Publication
(10) Pub. No.: US 2018/0220653 A1
(43) Pub. Date: Aug. 9, 2018

(54) Methods for Identifying Compounds that Directly Bind to Insect Transient Receptor Potential Channels

(71) Applicant: BASF SE, Ludwigshafen am Rhein (DE)

(72) Inventors: Alexandre Nesterov, Chapel Hill, NC (US); Ramani Kandasamy, Chapel Hill, NC (US); Wolfgang von Deyn, Neustadt (DE); Damian London, Wake Forest, NC (US); Katherine Lelito, Cary, NC (US); Vincent L Salgado, Durham, NC (US)

(73) Assignee: BASF SE, Ludwigshafen am Rhein (DE)

(21) Appl. No.: 15/737,170

(22) PCT Filed: Jun. 17, 2016
(86) PCT No.: PCT/EP2016/064078

§ 371 Date: Dec. 15, 2017

Related U.S. Application Data
(60) Provisional application No. 62/182,037, filed on Jun. 19, 2015.

(51) Int. Cl. A01N 43/54; A01N 43/707; A01N 43/90; G01N 33/68

(52) U.S. Cl. CPC A01N 43/54; A01N 43/707; G01N 2500/04; G01N 33/6872; G01N 2333/43552; A01N 43/90

(57) ABSTRACT

The present invention relates to a screening method for determining whether or not a candidate compound directly binds to an insect transient receptor potential V (TRPV) channel. The present invention further provides a method of identifying a compound that directly binds to a Nanchung protein, TRP and/or TRPV protein and/or channel using, a binding assay. The present invention further provides a method of classifying an insecticide compound by its mode of action using the binding assay and/or a functional assays.

### CROSS-REFERENCE TO RELATED APPLICATION

The present patent document claims the benefit of the filing date under 35 U.S.C. § 119(e) of Provisional U.S. Patent Application Ser. No. 62/182,037, filed Jun. 19, 2015, which is hereby incorporated by reference in its entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is PF78630_SEQLIST. The size of the text file is 619 KB, and the text file was created on Jun. 18, 2015.

### BACKGROUND

The present invention generally relates to methods for determining agents that bind directly to one or more Nanchung proteins, transient receptor potential (TRP) protein complexes and/or channels and/or transient receptor potential V (TRPV) protein complexes and/or channels. The present invention also relates to compositions and methods of insect control as well as mode of action classification for an insecticide composition comprising a compound capable of binding directly to the one or more Nanchung proteins, transient receptor potential (TRP) protein complexes and/or channels and/or transient receptor potential V (TRPV) protein complexes and/or channels. The present invention further relates to compositions and methods of insect control, as well as mode of action classification for an insecticide composition comprising a compound capable of directly binding to the one or more Nanchung proteins, transient receptor potential (TRP) protein complexes and/or channels and/or transient receptor potential V (TRPV) protein complexes and/or channels. The present invention even further relates to compositions and methods of insect control as well as mode of action classification for an insecticide composition comprising a compound capable of directly binding to the one or more Nanchung proteins, transient receptor potential (TRP) protein complexes and/or channels and/or transient receptor potential V (TRPV) protein complexes and/or channels wherein the compound is also capable of modulating the one or more TRP and/or TRPV channels.

Almost all field crops, plants, and commercial farming areas are susceptible to attacks by plant insect pests. Plant insect pests are a major factor in the loss of the world's commercially important agricultural crops resulting both, in economic hardship to farmers and nutritional deprivation for local populations in many parts of the world. In other words, insect pests cause great loses and damages to human agriculture, food supply, post-harvest storage, horticulture, animal health and public health.

While advances have been made in the control of these insects, these insects have been able to adopt and evade the control measures, resulting in resistant insect populations. As a result, there remains a need for better understanding of the mechanisms that underlie feeding and other behaviors of insect pests. Such knowledge would allow for the design of agents and strategies for intervening or preventing attacks on field crops, plants, and commercial farming areas by the insect pests.

Broad spectrum chemical pesticides and insecticides have been used extensively to control or eradicate plant insect pests of agricultural importance. There is, however, substantial interest in identifying and/or developing effective alternative insecticides. As such, effective methods for screening of the new insecticides would be desirable.

What are needed, then, are new methods that can be employed in screening for agents that modulate insect behavior, bind directly to one or more Nanchung proteins, transient receptor potential (TRP) and/or transient receptor potential V (TRPV) protein complexes and/or channels and in some cases, screening for agents that can act as insecticides.

### BRIEF SUMMARY OF THE INVENTION

In one embodiment, the present invention relates to a method for determining whether or not a candidate compound binds directly to one or more Nanchung proteins, transient receptor potential (TRP) and/or transient receptor potential V (TRPV) protein complexes and/or channels. The method may include providing one or more Nanchung proteins, transient receptor potential (TRP) and/or transient receptor potential V (TRPV) proteins, protein complexes and/or channels that may be located in a cell, derived from a cell, produced by a cell or produced in a cell-free system, contacting the one or more Nanchung proteins, TRP, and/or TRPV proteins, protein complexes and/or channels with a candidate compound, and assaying to determine the binding affinity of a candidate compound to the one or more Nanchung proteins, TRP, and/or TRPV proteins, protein complexes and/or channels, wherein the binding affinity of the candidate compound is indicative of the candidate compounds ability to directly bind to one or more Nanchung proteins, TRP, and/or TRPV proteins, protein complexes and/or channels.

For example, in certain embodiments, the binding assay step may include at least one of the following steps: (1) contacting the one or more Nanchung proteins, TRP, and/or TRPV proteins, protein complexes and/or channels with a compound, wherein the compound may be labeled or unlabeled; (2) measuring the concentration of the compound which is bound to the one or more Nanchung proteins, TRP, and/or TRPV proteins, protein complexes and/or channels; (3) calculating the equilibrium dissociation constant (K_{d}) for the one or more compounds; (4) wherein the K_{d} may be determined using one or more of the following methods: (I)(a) maximum binding (Bₘₐₓ) and (b) the concentration of labeled compound needed to reach 50% of maximum binding, (II) fluorescence polarization assay, (III) surface plasmon resonance, (IV) isothermal calorimetry, (V) Schild analysis; (5) contacting the one or more Nanchung proteins, TRP, and/or TRPV proteins, protein complexes and/or channels with a labeled compound having high affinity to the one or more Nanchung proteins, TRP, and/or TRPV proteins, protein complexes and/or channels; (6) contacting the one or more Nanchung proteins, TRP, and/or TRPV proteins, protein complexes and/or channels with a candidate compound in the presence of the labeled compound of step 1; (7) increasing the amount of the candidate compound available to contact the one or more Nanchung proteins, TRP, and/or TRPV proteins, protein complexes and/or channels until binding of the labeled compound is near 0%; (8) calculating the equilibrium inhibition constant (Kᵢ) and/or the equilibrium dissociation constant (K_{d}) for competition assay using one or more of the following methods: (I) (a) determining amount of bound labeled compound in the absence of the candidate compound and (b) determining concentration of the candidate compound that is required to reduce the amount of bound labeled compound by 50%, (II) fluorescent polarization assay, and/or (III) Schild analysis; (9) comparing the inhibition constant of the candidate compounds to other candidate compounds; (10) comparing the inhibition constant of the candidate compounds to the disassociation constant of the labeled compound.

In a second embodiment, the present invention relates to a method for determining the mode of action classification for the candidate compound according to the Insecticide Resistance Action Committee (IRAC) using one or more of the following: binding assay and/or functional assay. For example, in certain embodiments, a functional assay may be used to determine whether a candidate compound acts as a modulator of the one or more TRP and/or TRPV channels, and a binding assay may be used to determine if the candidate compound binds directly to the one or more Nanchung proteins, TRP and/or TRPV proteins, protein complexes, and/or channels and more particularly, to the same site on the one or more Nanchung proteins, TRP and/or TRPV proteins, protein complexes, and/or channels.

The functional assay may include at least one of the following steps: (1) detecting calcium ion mobilization in the first cell in response to the candidate compound, (2) detecting a membrane potential in the first cell in response to the candidate compound, (3) comparing calcium ion mobilization in the first cell in the absence of the candidate compound with calcium ion mobilization in the first cell in the presence of the candidate compound, (4) comparing a membrane potential in the first cell in the absence of the candidate compound with a membrane potential in the first cell in the presence of the candidate compound, (5) comparing calcium ion mobilization in the first cell in the presence of the candidate compound with calcium ion mobilization reference level indicative of no modulation of the TRPV channel, and/or (6) comparing a membrane potential in the first cell in the presence of the candidate compound with a membrane potential reference level indicative of no modulation of the TRPV channel. Preferably, the candidate compound may modulate the calcium ion mobilization or the membrane potential in the first cell by at least 20% relative to the reference level. The candidate compound may be a modulator that inhibits the activity of the insect TRPV channel. Alternatively, the candidate compound may be a modulator that activates the insect TRPV channel. The candidate compound may be a modulator that inhibits insect feeding behavior.

The binding assay may include at least one of the following steps: (1) contacting the one or more Nanchung proteins, TRP, and/or TRPV proteins, protein complexes and/or channels with a compound, wherein the compound may be labeled or unlabeled; (2) measuring the concentration of the compound which is bound to the one or more Nanchung proteins, TRP, and/or TRPV proteins, protein complexes and/or channels; (3) calculating the equilibrium disassociation constant (K_{d}) for the one or more compounds; (4) wherein the K_{d} may be determined using one or more of the following methods: (I)(a) maximum binding (Bₘₐₓ) and (b) the concentration of labeled compound needed to reach 50% of maximum binding, (II) fluorescence polarization assay, (III) surface plasmon resonance, (IV) isothermal calorimetry, (V) Schild analysis; (5) contacting the one or more Nanchung proteins, TRP, and/or TRPV proteins, protein complexes and/or channels with a labeled compound having high affinity to the one or more Nanchung proteins, TRP, and/or TRPV proteins, protein complexes and/or channels; (6) contacting the one or more one or more Nanchung proteins, TRP, and/or TRPV proteins, protein complexes and/or channels with a candidate compound in the presence of the labeled compound of step 1; (7) increasing the amount of the candidate compound available to contact the one or more Nanchung proteins, TRP, and/or TRPV proteins, protein complexes and/or channels until binding of the labeled compound is near 0%; (8) calculating the equilibrium inhibition constant (K) and/or the equilibrium dissociation constant (K_{d}) for competition assay using one or more of the following methods: (I) (a) determining amount of bound labeled compound in the absence of the candidate compound and (b) determining concentration of the candidate compound that is required to reduce the amount of bound labeled compound by 50%, (II) fluorescent polarization assay, and/or (III) Schild analysis; (9) comparing the inhibition constant of the candidate compounds to other candidate compounds; (10) comparing the inhibition constant of the candidate compounds to the disassociation constant of the labeled compound.

In the methods set forth herein, the one or more TRP channels, and/or TRPV channels may comprise one or more TRP proteins and/or one or more TRPV proteins in a complex. In the methods set forth herein, the one or more insect TRP channels, and/or TRPV channels may comprise one or more Nanchung proteins, Inactive proteins, TRP proteins, TRPV proteins, Nanchung and Inactive protein complexes, Nanchung and TRPV protein complexes, and/or Nanchung and TRP protein complexes. For clarity, the TRP and/or TRPV channels may also comprise a Nanchung protein and any other TRP channel proteins found in cellular membranes, inserted in artificial membranes and/or a cell-free system. It is to be understood that the Nanchung, TRP and/or TRPV proteins and/or protein complexes may be found in cellular membranes, in solution, or in a cell-free system. It is too be understood that the TRP and/or TRPV channels may be contained within one or more cellular membranes, one or more pieces of cellular membranes, produced in a cellular system, produced in a cell-free system, and/or from cultured cells in a petri dish, or from cells or tissues derived from experimental animals or experimental insects. For purposes of determining whether a candidate compound directly binds to the insect TRP and/or TRPV channels, the TRP and/or TRPV channels may comprise one or more Nanchung, TRP and/or TRPV protein complexes.

It is to be understood that Nanchung and Inactive proteins are both insect TRPV proteins. However, Nanchung may also form a complex with TRP proteins. The ratio of the Nanchung to Inactive proteins co-expressed in the TRP and/or TRPV complex is, preferably, about 3:1 to about 1:3, more preferably about 1:1. The ratio of the Nanchung to TRP proteins co-expressed in the TRP and/or TRPV complex is, preferably, about 3:1 to about 1:3, more preferably about 1:1. The candidate compound may be a small organic molecule, small inorganic molecule, polysaccharides, peptides, proteins, nucleic acids, an extract made from biological materials, and any combination thereof.

The method may further include one or more of these additional steps: providing one or more mammalian TRP channels; contacting the one or more mammalian TRP and/or TRPV channels with a labeled compound and/or a candidate compound, wherein the labeled compound may be the candidate compound or a separate labeled compound; assaying to determine binding affinity of the labeled compound and/or the candidate compound to the one or more mammalian TRP and/or TRPV channels, wherein little to no binding affinity to the one or more mammalian TRP and/or TRPV channel identifies the candidate compound as a selectively binding to the one or more Nanchung proteins, insect TRP channels and/or TRPV channels. The insect may be an agricultural/horticultural pest or a disease vector or a parasite.

In another embodiment, the methods set forth above may use one or more of the following compounds as the labeled compound: afidopyropen, pymetrozine, and/or N-deacetylated form of pyrifluquinazon.

In yet another embodiment, the present invention relates to a candidate compound selected by the methods described above. The candidate compound may be a modulator that inhibits or increases the activity of the insect TRPV channel. The candidate compound may be a modulator that inhibits a feeding behavior of an insect. Even further, the candidate compound may modulate and/or bind directly to the one or more Nanchung proteins, TRP and/or TRPV proteins, protein complexes and/or channels, and in particular to the same site on the one or more Nanchung proteins, TRP and/or TRPV proteins, protein complexes and/or channels. The candidate compound may further modulate and/or bind directly to one or more insect Nanchung proteins, insect TRP and/or insect TRPV proteins, protein complexes and/or channels, and in particular to the same site on the one or more Nanchung proteins, TRP and/or TRPV proteins, protein complexes and/or channels. The candidate compound may further modulate and/or bind directly to one or more arthropod Nanchung proteins, arthropod TRP and/or arthropod TRPV proteins, protein complexes and/or channels, and in particular to the same site on the one or more Nanchung proteins, TRP and/or TRPV proteins, protein complexes and/or channels. The candidate compound may further modulate and/or bind directly to one or more Mollusk Nanchung proteins, Mollusk TRP and/or Mollusk TRPV proteins, protein complexes and/or channels, and in particular to the same site on the one or more Nanchung proteins, TRP and/or TRPV proteins, protein complexes and/or channels. The candidate compound may further modulate and/or bind directly to one or more Nematode Nanchung proteins, Nematode TRP and/or Nematode TRPV protein complexes and/or channels, and in particular to the same site on the one or more Nanchung proteins, TRP and/or TRPV proteins, protein complexes and/or channels. The candidate compound may further modulate and/or bind directly to one or more pest Nanchung proteins, pest TRP and/or TRPV proteins protein complexes and/or channels. The candidate compound may further be unable to modulate and/or bind directly to one or more Mammalian Nanchung proteins, Mammalian TRP and/or Mammalian TRPV protein complexes and/or channels and/or other non-target/non-pest species.

In another embodiment, the present invention relates to a composition containing the candidate compound identified by one or more of the methods set forth herein.

In another embodiment, the present invention relates to a method of insect control that includes applying to an insect or to an insects habitat, feeding source, and/or environment/habitat a composition comprising the candidate compound selected by one or more of the methods described herein or a compound selected by one or more of the methods described above. The compound may be an inhibitor of an insect TRP and/or TRPV channel. The compound may be an activator of an insect TRP and/or TRPV channel. The compound may bind directly to the insect Nanchung protein, TRP and/or TRPV channel. The insect may be an agricultural/horticultural pest or a disease vector or a parasite.

### BRIEF DESCRIPTION OF THE DRAWINGS

FIG. 1 depicts molecular structures of afidopyropen, pymetrozine (PM), pyrifluquinazon (PFQ) and its N-deacetylated form (dPFQ).

FIG. 2 depicts the effects of afidopyorpen and pymetrozine (PM) in American grasshoppers (Schistocerca Americana)

FIG. 3 depict activation of Ca²⁺ mobilization by afidopyropen, pymetrozine (PM), and N-deacetylated form of pyrifluquinazon (dPFQ) in mammalian cells co-expressing Nan and lay, but not in parental hamster cells (cells are cells prior to infection with the genes or proteins of interest), nor in cells expressing Nanchung or Inactive alone.

FIG. 4 depicts graphs demonstrating that afidopyropen, pymetrozine (PM), and N-deacetylated form of pyrifluquinazon (dPFQ) evoke membrane depolarization response in mammalian cells co-expressing Nanchung and Inactive, but not in parental hamster cells, nor in cells expressing Nanchung or Inactive alone.

FIG. 4a depicts graphs demonstrating that N-deacetylated form of pyrifluquinazon (dPFQ) evokes current in frog oocytes co-expressing Nanchung and Inactive, but not in oocytes expressing Nanchung or Inactive alone.

FIG. 5 depicts dose response relationships of calcium responses evoked by afidopyropen, pymetrozine (PM), and N-deacetylated form of pyrifluquinazon (dPFQ) in cells co-expressing Nanchung and Inactive.

FIG. 6 depicts afidopyropen not activating mammalian TRRV4 channel.

FIG. 7 depicts specific binding of [³H]-afidopyropen to membranes co-expressing Nan and lay.

FIG. 8A depicts [³H]-afidopyropen saturation binding to Nan protein and increased binding affinity in the presence of Iav

FIG. 8B depicts Scatchard transformation of the saturation binding identified in FIG. 8A.

FIG. 9A. Both pymetrozine (PM) and N-deacetylated form of pyrifluquinazon (dPFQ) compete with [³H]-afidopyropen for binding to Nan+Iav membranes.

FIG. 9B. Both pymetrozine (PM) and N-deacetylated form of pyrifluquinazon (dPFQ) compete with [³H]-afidopyropen from binding to Nan only membranes.

[SN1][SL2]FIGS. 10-36 correspond to SEQ ID NOS: 1-27, respectively, and provide the nucleotide sequences of a Nanchung (Nan) gene from different species having the accession number specified in the Figure.

FIGS. 37-63 correspond to SEQ ID NOS: 54-80, respectively, and provide the amino acid sequences of a Nanchung (Nan) protein having the accession numbers specified in the Figure.

FIGS. 64-89 correspond to SEQ ID NOS: 28-53, respectively, and provide the nucleotide sequences of an Inactive (Iav) gene from different species having the accession numbers specified in the Figure.

FIGS. 90-115 correspond to SEQ ID NOS: 81-106, respectively, and provide the amino acid sequence of an Inactive (Iav) protein from different species having the accession numbers specified in the Figure.

FIG. 116 depicts a sequence alignment of the Nan proteins (SEQ ID NOS 59, 60, 63, 58, 57, 56, 54, 74, 73, 75, 66, 70, 110, 68, and 111, residues 1-448 and 560-900 of SEQ ID NO: 64, and SEQ ID NOS 76, 79, 62, 77, 72, 78, 71, 80, 65, and 61, all respectively, in order of appearance).

FIG. 117 depicts sequence alignment of the Iav proteins (SEQ ID NOS 84 and 85, residues 1-916, 954-1102, and 1167-1216 of SEQ ID NO: 86, and SEQ ID NOS 83, 82, 81, 101, 104, 100, 99, 92, 98, 93, 90, 89, 94, 95, 88, 102, 112, 103, 105, 106, 96, 87, and 91, all respectively, in order of appearance).

FIG. 118 depicts an alignment tree for Nan proteins.

FIG. 119 depicts an alignment tree for Iav proteins.

### DETAILED DESCRIPTION OF THE DRAWINGS AND THE PRESENTLY PREFERRED EMBODIMENTS

### I. General Considerations

The transient receptor potential (TRP) channels constitute a large and important class of channels involved in modulating cellular homeostasis. The present invention provides methods and compositions that identify or include one or more compounds that bind to and/or modulate at least one TRP family member, including, but not limited to a TRPV channel in an insect.

Specifically, the present invention provides methods for identifying candidate compounds capable of directly binding to one or more insect Nanchung proteins, TRP and/or TRPV proteins, protein complexes and/or channels and/or modulating one or more insect TRP and/or TRPV channel. Modulating an insect TRP and/or TRPV channel may modulate calcium homeostasis, sodium homeostasis, intracellular calcium levels, membrane polarization (resting membrane potential), and/or cation levels in a cell. Compounds that can modulate one or more insect TRP and/or TRPV channels are useful in many aspects including, but not limited to, insect control. Knowing that a compound binds directly to an insect TRP and/or TRPV channel is critical for insect management planning and preventing the development of resistant insect populations.

The present invention further identifies afidopyropen, PM, and/or dPFQ as compounds having high affinity to Nanchung protein, TRP, and/or TRPV proteins, protein complexes, and/or channels, such that one or more of the compounds above may be used in the methods presented herein as a labeled compound for purposes of the competition binding assay set forth herein.

The present invention also provides compositions comprising the candidate compounds identified herein for use as insecticides for treating pests. The present invention further comprises classifying the candidate compounds identified herein by their mode of action.

Without being bound by theory, the modulation of an insect TRP and/or TRPV elicits a signaling pathway that brings forth sensory neuron modulation which may increase or decrease feeding behavior of an insect, and such modulation (e.g., activation) of an insect TRP and/or TRPV channel in an insect may lead to a decrease in feeding behavior of such insect leading to the insect's death by starvation. Thus, again without being bound by theory, it is believed that compounds that modulate (e.g., activate) an insect TRP and/or TRPV channel may be used as insecticides.

TRP channels have been classified into at least seven groups: TRPC (canonical), TRPV (vanilloid), TRPM (melastatin), TRPP (polycystins), TRPML (mucolipins), TRPA (ankyrin), and TRPN (no mechanosensor potential C), which is not found in humans.

The TRPC group can be divided into 4 subfamilies (TRPC1, TRPC4,5, TRPC3,6,7 and TRPC2) based on sequence homology and functional similarities.

Currently, the mammalian TRPV family has 6 members. TRPV5 and TRPV6 are more closely related to each other than to TRPV1, TRPV2, TRPV3, or TRPV4. TRPA1 is most closely related to TRPV3, and is more closely related to TRPV1 and TRPV2 than to TRPV5 and TRPV6. The insects have only two members of TRPV family: Nanchung (Nan), and Inactive (Iav), which are most closely related to mammalian TRPV5 and TRPV6. The TRPV is expressed in a great number of organisms, including, e.g., insects (Drosophila, Tribolium, Pediculus, Culex, and Anopheles), crustaceans (Daphnia pulex) nematodes (Caenorhabditis elegans), molluscs (Aplysia californica, Lottia gigantea) and mammals (humans, mice, rats, monkeys and chimpanzee.

Mollusks, nemotodes, Arthropod, and more particularly insect, TRPV channels are represented by Nanchung and/or Inactive proteins (or homologues of thereof). Nanchung and Inactive proteins form a complex (Nesterov et al. 2015), and are implicated in insect hearing (Matsuura et al., 2009). However, Nanchung may form a complex with other TRP proteins

The TRPM family has 8 members. Constituents include the following: the founding member TRPM1 (Melastatin or LTRPC1), TRPM3 (KIAA1616 or LTRPC3), TRPM7 (TRP-PLIK, ChaK(1), LTRPC7), TRPM6 (ChaK2), TRPM2 (TRPC7 or LTRPC2), TRPM8 (Trp-p8 or CMR1), TRPM5 (Mtr1 or LTRPC5), and TRPM4 (FLJ20041 or LTRPC4).

The sole mammalian member of the TRPA family is TRPA1, whereas insects contain at least four members of this family: TRPA1, Painless, Pyrexia, and Waterwitch.

The TRPML family consists of the mucolipins, which include TRPML1 (mucolipins 1), TRPML2 (mucolipins 2), and TRPML3 (mucolipin3).

The TRPP family consists of two groups of channels: those predicted to have six transmembrane domains and those that have 11. TRPP2 (PKD2), TRPP3 (PKD2L1), TRPP5 (PKD2L2) are all predicted to have six transmembrane domains. TRPP1 (PKD1, PC1), PKD-REJ and PKD-1L1 are all thought to have 11 transmembrane domains. Insects have only one member of TRPP family, which is absent in some species.

The inventors have discovered that afidopyropen binds directly to the Nanchung protein alone or in complex with other TRP and/or TRPV proteins. Furthermore, the inventors have developed methods of identifying additional compounds that bind directly to the insect Nanchung protein, TRPV protein complex or related insect TRP protein complex having a Nanchung protein incorporated in its complex. Even further the inventors have identified that afidopyropen is a compound that is useful for identifying candidate compounds capable of binding directly to the insect Nanchung protein, TRPV protein complex or related insect TRP protein complex having a Nanchung protein incorporated in its complex. Identification of such compounds may lead to the identification of suitable chemical compounds that can function as insecticides and be useful in a pest management program.

Even further the inventors discovered that some compounds have increased binding affinity when both the Nanchung protein and the Inactive protein were present in the protein complex. Accordingly, it may be beneficial to identify compounds that directly bind to one or more TRP and or TRPV protein complexes comprising one or more Nanchung proteins. Additionally, it may be beneficial to identify compounds that bind directly to one or more TRP protein complexes comprising one or more Nanchung proteins and one or more Inactive proteins. It may also be beneficial to identify compounds that bind directly to one or more Nanchung proteins, and/or TRP and/or TRPV protein complexes and or channels. It may be beneficial to use the identified compounds as insecticides. It may be further beneficial to identify compound that directly bind to one or more Nanchung proteins, and/or TRP and/or TRPV proteins, protein complexes and/or channels and that may be found to modulate the TRP and/or TRPV channels of pest species while not binding directly to and/or modulating the one or more Nanchung proteins, and/or TRP and/or TRPV proteins, protein complexes and/or channels of mammalians and/or other non-pest species.

### II. Definitions

Unless defined otherwise, all technical and scientific terms used herein have the same meaning as commonly understood to one of ordinary skill in the art. Although any methods, devices, and materials similar or equivalent to those described herein can be used in the practice or testing of the presently disclosed subject matter, representative methods, devices, and materials are now described.

The terms “a,” “an,” and “the” refer to “one or more” when used in this application, including the claims. Thus, for example, reference to “a cell” (e.g., “a mammalian cell”) includes a plurality of such cells (e.g., a plurality of mammalian cells in culture).

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The terms “insect,” “insect pest,” or “plant insect pest” refer to any one of the numerous usually small arthropod animals of the class Insecta that are known to associate with plants and which, as a result of that association, causes a detrimental effect on the plant's health and vigor. The term plant as used herein encompasses whole plants and parts of plants such as roots, stems, leaves and seed, as well as cells and tissues within the plants or plant parts. The terms “insect,” “insect pest,” or “plant insect pest” are used interchangeably throughout the instant application.

The terms “pests” and/or “pest species” refer to any one of numerous usually small arthropod animals of the class Insecta that are known to associate with plants and which, as a result of that association, causes a detrimental effect on the plant's health and vigor. Additionally, the terms refer to include mollusk, nematodes, other arthropod classes that are known to associate with plants and which, as a result of that association, causes a detrimental effect on the plant's health and vigor. The terms further includes arthropods, gastropods and nematodes including but not limited to: insects from the order of Lepidoptera, for example Achroia grisella, Acleris spp. such as A. fimbriana, A. gloverana, A. variana; Acrolepiopsis assectella, Acronicta major, Adoxophyes spp. such as A. cyrtosema, A. orana; Aedia leucomelas, Agrotis spp. such as A. exclamationis, A. fucosa, A. ipsilon, A. orthogoma, A. segetum, A. subterranea; Alabama argillacea, Aleurodicus dispersus, Alsophila pometaria, Ampelophaga rubiginosa, Amyelois transitella, Anacampsis sarcitella, Anagasta kuehniella, Anarsia lineatella, Anisota senatoria, Antheraea pernyi, Anticarsia (=Thermesia) spp. such as A. gemmatalis; Apamea spp, Aproaerema modicella, Archips spp. such as A. argyrospila, A. fuscocupreanus, A. rosana, A. xyloseanus; Argyresthia conjugella, Argyroploce spp., Argyrotaenia spp. such as A. velutinana; Athetis mindara, Austroasca viridigrisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp. such as C. murinana, C. podana; Cactoblastis cactorum, Cadra cautella, Calingo braziliensis, Caloptilis theivora, Capua reticulana, Carposina spp. such as C. niponensis, C. sasakii; Cephus spp, Chaetocnema aridula, Cheimatobia brumata, Chilo spp. such as C. Indicus, C. suppressalis, C. partellus; Choreutis pariana, Choristoneura spp. such as C. conflictana, C. fumiferana, C. longicellana, C. murinana, C. occidentalis, C. rosaceana; Chrysodeixis (=Pseudoplusia) spp. such as C. eriosoma, C. includens; Cirphis unipuncta, Clysia ambiguella, Cnaphalocerus spp., Cnaphalocrocis medinalis, Cnephasia spp., Cochylis hospes, Coleophora spp., Colias eurytheme, Conopomorpha spp., Conotrachelus spp., Copitarsia spp., Corcyra cephalonica, Crambus caliginosellus, Crambus teterrellus, Crocidosema (=Epinotia) aporema, Cydalima (=Diaphania) perspectalis, Cydia (=Carpocapsa) spp. such as C. pomonella, C. latiferreana; Dalaca noctuides, Datana integerrima, Dasychira pinicola, Dendrolimus spp. such as D. pini, D. spectabilis, D. sibiricus; Desmia funeralis, Diaphania spp. such as D. nitidalis, D. hyalinata; Diatraea grandiosella, Diatraea saccharalis, Diphthera festiva, Earias spp. such as E. insulana, E. vittella; Ecdytolopha aurantianu, Egira (=Xylomyges) curialis, Elasmopalpus lignosellus, Eldana saccharina, Endopiza viteana, Ennomos subsignaria, Eoreuma loftini, Ephestia spp. such as E. cautella, E. elutella, E. kuehniella; Epinotia aporema, Epiphyas postvittana, Erannis tiliaria, Erionota thrax, Etiella spp., Eulia spp., Eupoecilia ambiguella, Euproctis chrysorrhoea, Euxoa spp., Evetria bouliana, Faronta albilinea, Feltia spp. such as F. subterranean; Galleria mellonella, Gracillaria spp., Grapholita spp. such as G. funebrana, G. molesta, G. inopinata; Halysidota spp., Harrisina americana, Hedylepta spp., Helicoverpa spp. such as H. armigera (=Heliothis armigera), H. zea (=Heliothis zea); Heliothis spp. such as H. assulta, H. subflexa, H. virescens; Hellula spp. such as H. undalis, H. rogatalis; Helocoverpa gelotopoeon, Hemileuca oliviae, Herpetogramma licarsisalis, Hibernia defoliaria, Hofinannophila pseudospretella, Homoeosoma electellum, Homona magnanima, Hypena scabra, Hyphantria cunea, Hyponomeuta padella, Hyponomeuta malinellus, Kakivoria flavofasciata, Keiferia lycopersicella, Lambdina fiscellaria fiscellaria, Lambdina fiscellaria lugubrosa, Lamprosema indicata, Laspeyresia molesta, Leguminivora glycinivorella, Lerodea eufala, Leucinodes orbonalis, Leucoma salicis, Leucoptera spp. such as L. coffeella, L. scitella; Leuminivora lycinivorella, Lithocolletis blancardella, Lithophane antennata, Llattia octo (=Amyna axis), Lobesia botrana, Lophocampa spp., Loxagrotis albicosta, Loxostege spp. such as L. sticticalis, L. cereralis; Lymantria spp. such as L. dispar, L. monacha; Lyonetia clerkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quinquemaculata, M. sexta; Marasmia spp, Marmara spp., Maruca testulalis, Megalopyge lanata, Melanchra picta, Melanitis leda, Mocis spp. such as M. lapites, M. repanda; Mocis latipes, Monochroa fragariae, Mythimna separata, Nemapogon cloacella, Neoleucinodes elegantalis, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp. such as O. nubilalis; Oulema oryzae, Paleacrita vernata, Panolis flammea, Parnara spp., Papaipema nebris, Papilio cresphontes, Paramyelois transitella, Paranthrene regalis, Paysandisia archon, Pectinophora spp. such as P. gossypiella; Peridroma saucia, Perileucoptera spp., such as P. coffeella; Phalera bucephala, Phryganidia californica, Phthorimaea spp. such as P. operculella; Phyllocnistis citrella, Phyllonorycter spp. such as P. blancardella, P. crataegella, P. issikii, P. ringoniella; Pieris spp. such as P. brassicae, P. rapae, P. napi; Pilocrocis tripunctata, Plathypena scabra, Platynota spp. such as P. flavedana, P. idaeusalis, P. stultana; Platyptilia carduidactyla, Plebejus argus, Plodia interpunctella, Plusia spp, Plutella maculipennis, Plutella xylostella, Pontia protodica, Prays spp., Prodenia spp., Proxenus lepigone, Pseudaletia spp. such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp. such as S. incertulas, S. innotata; Scotia segetum, Sesamia spp. such as S. inferens, Seudyra subflava, Sitotroga cerealella, Sparganothis pilleriana, Spilonota lechriaspis, S. ocellana, Spodoptera (=Lamphygma) spp. such as S. eridania, S. exigua, S. frugiperda, S. latisfascia, S. littoralis, S. litura, S. omithogalli; Stigmella spp., Stomopteryx subsecivella, Strymon bazochii, Sylepta derogata, Synanthedon spp. such as S. exitiosa, Tecia solanivora, Telehin licus. Thaumatopoea pityocampa, Thaumatotibia (=Cryptophlebia) leucotreta, Thaumetopoea pityocampa, Thecla spp., Theresimima ampelophaga, Thyrinteina spp, Tildenia inconspicuella, Tinea spp. such as T. cloacella, T. pellionella; Tineola bisselliella, Tortrix spp. such as T. viridana; Trichophaga tapetzella, Trichoplusia spp. such as T. ni; Tuta (=Scrobipalpula) absoluta, Udea spp. such as U. rubigalis, U. rubigalis; Virachola spp, Yponomeuta padella, and Zeiraphera canadensis; insects from the order of Coleoptera, for example Acalymma vittatum, Acanthoscehdes obtectus, Adoretus spp., Agelastica alni, Agrilus spp. such as A. anxius, A. planipennis, A. sinuatus; Agriotes spp. such as A. fuscicollis, A. lineatus, A. obscurus; Alphitobius diaperinus, Amphimallus solstitialis, Anisandrus dispar, Anisoplia austriaca, Anobium punctatum, Anomala corpulenta, Anomala rufocuprea, Anoplophora spp. such as A. glabripennis; Anthonomus spp. such as A. eugenii, A. grandis, A. pomorum; Anthrenus spp., Aphthona euphoridae, Apion spp., Apogonia spp., Athous haemorrhoidalis, Atomaria spp. such as A. linearis; Attagenus spp., Aulacophora femoralis, Blastophagus piniperda, Blitophaga undata, Bruchidius obtectus, Bruchus spp. such as B. lentis, B. pisorum, B. rufimanus; Byctiscus betulae, Callidiellum rufipenne, Callopistria floridensis, Callosobruchus chinensis, Cameraria ohridella, Cassida nebulosa, Cerotoma trifurcata, Cetonia aurata, Ceuthorhynchus spp. such as C. assimilis, C. napi; Chaetocnema tibialis, Cleonus mendicus, Conoderus spp. such as C. vespertinus; Conotrachelus nenuphar, Cosmopolites spp., Costelytra zealandica, Crioceris asparagi, Cryptolestes ferrugineus, Cryptorhynchus lapathi, Ctenicera spp. such as C. destructor; Curculio spp., Cylindrocopturus spp., Cyclocephala spp, Dactylispa balyi, Dectes texanus, Dermestes spp., Diabrotica spp. such as D. undecimpunctata, D. speciosa, D. longicornis, D. semipunctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. similaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp. such as H. brunneipennis, H. postica; Hypomeces squamosus, Hypothenemus spp., Ips typographus, Lachnosterna consanguinea, Lasioderma serricorne, Latheticus oryzae, Lathridius spp., Lema spp. such as L. bilineata, L. melanopus; Leptinotarsa spp. such as L. decemlineata; Leptispa pygmaea, Limonius californicus, Lissorhoptrus oryzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus; Melolontha spp. such as M. hippocastani, M. melolontha; Metamasius hemipterus, Microtheca spp, Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp. such as P. brassicae, P. cochleariae; Phoracantha recurva, Phyllobius pyri, Phyllopertha horticola, Phyllophaga spp. such as P. helleri; Phyllotreta spp. such as P. chrysocephala, P. nemorum, P. striolata, P. vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. billineatus, R. ferrugineus, R. palmarum, R. phoenicis, R. vulneratus; Saperda candida, Scolytus schevyrewi, Scyphophorus acupunctatus, Sitona lineatus, Sitophilus spp. such as S. granaria, S. oryzae, S. zeamais; Sphenophorus spp. such as S. levis; Stegobium paniceum, Sternechus spp. such as S. subsignatus; Strophomorphus ctenotus, Symphyletes spp., Tanymecus spp., Tenebrio molitor, Tenebrioides mauretanicus, Tribolium spp. such as T. castaneum; Trogoderma spp., Tychius spp., Xylotrechus spp. such as X. pyrrhoderus; and, Zabrus spp. such as Z. tenebrioides; insects from the order of Diptera for example Aedes spp. such as A. aegypti, A. albopictus, A. vexans; Anastrepha ludens, Anopheles spp. such as A. albimanus, A. crucians, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria; Chrysops atlanticus, Chrysops discalis, Chrysops silacea, Cochliomyia spp. such as C. hominivorax; Contarinia spp. such as C. sorghicola; Cordylobia anthropophaga, Culex spp. such as C. nigripalpus, C. pipiens, C. quinquefasciatus, C. tarsalis, C. tritaeniorhynchus; Culicoides furens, Culiseta inornata, Culiseta melanura, Cuterebra spp., Dacus cucurbitae, Dacus oleae, Dasineura brassicae, Dasineura oxycoccana, Delia spp. such as D. antique, D. coarctata, D. platura, D. radicum; Dermatobia hominis, Drosophila spp. such as D. suzukii, Fannia spp. such as F. canicularis; Gastraphilus spp. such as G. intestinalis; Geomyza tipunctata, Glossina spp. such as G. fuscipes, G. morsitans, G. palpalis, G. tachinoides; Haematobia irritans, Haplodiplosis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii; Lucilia spp. such as L. caprina, L. cuprina, L. sericata; Lycoria pectoralis, Mansonia titillanus, Mayetiola spp. such as M. destructor; Musca spp. such as M. autumnalis, M. domestica; Muscina stabulans, Oestrus spp. such as O. ovis; Opomyza florum, Oscinella spp. such as O. frit; Orseolia oryzae, Pegomya hysocyami, Phlebotomus argentipes, Phorbia spp. such as P. antiqua, P. brassicae, P. coarctata; Phytomyza gymnostoma, Prosimulium mixtum, Psila rosae, Psorophora columbiae, Psorophora discolor, Rhagoletis spp. such as R. cerasi, R. cingulate, R. indifferens, R. mendax, R. pomonella; Rivellia quadrifasciata, Sarcophaga spp. such as S. haemorrhoidalis; Simulium vittatum, Sitodiplosis mosellana, Stomoxys spp. such as S. calcitrans; Tabanus spp. such as T. atratus, T. bovinus, T. lineola, T. similis; Tannia spp., Thecodiplosis japonensis, Tipula oleracea, Tipula paludosa, and Wohlfahrtia spp.; insects from the order of Thysanoptera for example, Baliothrips biformis, Dichromothrips corbetti, Dichromothrips ssp., Echinothrips americanus, Enneothrips flavens, Frankliniella spp. such as F. fusca, F. occidentalis, F. tritici; Heliothrips spp., Hercinothrips femoralis, Kakothrips spp., Microcephalothrips abdominalis, Neohydatothrips samayunkur, Pezothrips kellyanus, Rhipiphorothrips cruentatus, Scirtothrips spp. such as S. citri, S. dorsalis, S. perseae; Stenchaetothrips spp, Taeniothrips cardamoni, Taeniothrips inconsequens, Thrips spp. such as T. imagines, T. hawaiiensis, T. oryzae, T. palmi, T. parvispinus, T. tabaci; insects from the order of Hemiptera for example, Acizzia jamatonica, Acrosternum spp. such as A. hilare; Acyrthosipon spp. such as A. onobrychis, A. pisum; Adelges laricis, Adelges tsugae, Adelphocoris spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrixus spp., Amrasca spp., Anasa tristis, Antestiopsis spp., Anuraphis cardui, Aonidiella spp., Aphanostigma pini, Aphidula nasturtii, Aphis spp. such as A. craccivora, A. fabae, A. forbesi, A. gossypii, A. grossulariae, A. maidiradicis, A. pomi, A. sambuci, A. schneideri, A. spiraecola; Arboridia apicalis, Arilus critatus, Aspidiella spp., Aspidiotus spp., Atanus spp., Aulacaspis yasumatsui, Aulacorthum solani, Bactericera cockerelli (Paratrioza cockerelli), Bemisia spp. such as B. argentifolii, B. tabaci (Aleurodes tabaci); Blissus spp. such as B. leucopterus; Brachycaudus spp. such as B. cardui, B. helichrysi, B. persicae, B. prunicola; Brachycolus spp., Brachycorynella asparagi, Brevicoryne brassicae, Cacopsylla spp. such as C. fulguralis, C. pyricola (Psylla pini); Calligypona marginata, Calocoris spp., Campylomma livida, Capitophorus horni, Carneocephala fulgida, Cavelerius spp., Ceraplastes spp., Ceratovacuna lanigera, Ceroplastes ceriferus, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as C. hemipterus, C. lectularius; Coccomytilus halli, Coccus spp. such as C. hesperidum, C. pseudomagnoliarum; Corythucha arcuata, Creontiades dilutus, Cryptomyzus ribis, Chrysomphalus aonidum, Cryptomyzus ribis, Ctenarytaina spatulata, Cyrtopeltis notatus, Dalbulus spp., Dasynus piperis, Dialeurodes spp. such as D. citrifolii; Dalbulus maidis, Diaphorina spp. such as D. citri; Diaspis spp. such as D. bromeliae; Dichelops furcatus, Diconocoris hewetti, Doralis spp., Dreyfusia nordmannianae, Dreyfusia piceae, Drosicha spp., Dysaphis spp. such as D. plantaginea, D. pyri, D. radicola; Dysaulacorthum pseudosolani, Dysdercus spp. such as D. cingulatus, D. intermedius; Dysmicoccus spp., Edessa spp, Geocoris spp, Empoasca spp. such as E. fabae, E. solana; Epidiaspis leperii, Eriosoma spp. such as E. lanigerum, E. pyricola; Erythroneura spp., Eurygaster spp. such as E. integriceps; Euscelis bilobatus, Euschistus spp. such as E. heros, E. impictiventris, E. servus; Fiorinia theae, Geococcus coffeae, Glycaspis brimblecombei, Halyomorpha spp. such as H. halys; Heliopeltis spp., Homalodisca vitripennis (=H. coagulata), Horcias nobilellus, Hyalopterus pruni, Hyperomyzus lactucae, Icerya spp. such as I. purchase; Idiocerus spp., Idioscopus spp., Laodelphax striatellus, Lecanium spp., Lecanoideus floccissimus, Lepidosaphes spp. such as L. ulmi; Leptocorisa spp., Leptoglossus phyllopus, Lipaphis erysimi, Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbiae; Macrosteles quadrilineatus, Mahanarva fimbriolata, Megacopta cribraria, Megoura viciae, Melanaphis pyrarius, Melanaphis sacchari, Melanocallis (=Tinocallis) caryaefoliae, Metcafiella spp., Metopolophium dirhodum, Monellia costalis, Monelliopsis pecanis, Myzocallis coryli, Murgantia spp, Myzus spp. such as M. ascalonicus, M. cerasi, M. nicotianae, M. persicae, M. varians; Nasonovia ribis-nigri, Neotoxoptera formosana, Neomegalotomus spp, Nephotettix spp. such as N. malayanus, N. nigropictus, N. parvus, N. virescens; Nezara spp. such as N. viridula; Nilaparvata lugens, Nysius huttoni, Oebalus spp. such as O. pugnax; Oncometopia spp., Orthezia praelonga, Oxycaraenus hyalinipennis, Parabemisia myricae, Parlatoria spp., Parthenolecanium spp. such as P. corni, P. persicae; Pemphigus spp. such as P. bursarius, P. populivenae; Peregrinus maidis, Perkinsiella saccharicida, Phenacoccus spp. such as P. aceris, P. gossypii; Phloeomyzus passerinii, Phorodon humuli, Phylloxera spp. such as P. devastatrix, Piesma quadrata, Piezodorus spp. such as P. guildinii; Pinnaspis aspidistrae, Planococcus spp. such as P. citri, P. ficus; Prosapia bicincta, Protopulvinaria pyriformis, Psallus seriatus, Pseudacysta persea, Pseudaulacaspis pentagona, Pseudococcus spp. such as P. comstocki; Psylla spp. such as P. mali; Pteromalus spp., Pulvinaria amygdali, Pyrilla spp., Quadraspidiotus spp., such as Q. perniciosus; Quesada gigas, Rastrococcus spp., Reduvius senilis, Rhizoecus americanus, Rhodnius spp., Rhopalomyzus ascalonicus, Rhopalosiphum spp. such as R. pseudobrassicas, R. insertum, R. maidis, R. padi; Sagatodes spp., Sahlbergella singularis, Saissetia spp., Sappaphis mala, Sappaphis mali, Scaptocoris spp, Scaphoides titanus, Schizaphis graminum, Schizoneura lanuginosa, Scotinophora spp., Selenaspidus articulatus, Sitobion avenae, Sogata spp., Sogatella furcifera, Solubea insularis, Spissistilus festinus (=Stictocephala festina); Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomaspis spp., Toxoptera spp. such as T. aurantii; Trialeurodes spp. such as T. abutilonea, T. ricini, T. vaporariorum; Triatoma spp., Trioza spp., Typhlocyba spp., Unaspis spp. such as U. citri, U. yanonensis; and Viteus vitifolii; insects from the order Hymenoptera for example Acanthomyops interjectus, Athalia rosae, Atta spp such as A. capiguara, A. cephalotes, A. cephalotes, A. laevigata, A. robusta, A. sexdens, A. texana, Bombus spp., Brachymyrmex spp., Camponotus spp such as C. floridanus, C. pennsylvanicus, C. modoc; Cardiocondyla nuda, Chalibion sp, Crematogaster spp., Dasymutilla occidentalis, Diprion spp., Dolichovespula maculata, Dorymyrmex spp, Dryocosmus kuriphilus, Formica spp, Hoplocampa spp. such as H. minuta, H. testudinea; Iridomyrmex humilis, Lasius spp. such as L. niger, Linepithema humile, Liometopum spp, Leptocybe invasa, Monomorium spp such as M. pharaonis, Monomorium, Nylandria fulva, Pachycondyla chinensis, Paratrechina longicomis, Paravespula spp such as P. germanica, P. pennsylvanica, P. vulgaris; Pheidole spp such as P. megacephala; Pogonomyrmex spp such as P. barbatus, P. califomicus, Polistes rubiginosa, Prenolepis impairs, Pseudomyrmex gracilis, Schelipron spp, Sirex cyaneus, Solenopsis spp such as S. geminata, S. invicta, S. molesta, S. richteri, S. xyloni, Sphecius speciosus, Sphex spp, Tapinoma spp such as T. melanocephalum, T. sessile; Tetramorium spp such as T. caespitum, T. bicarinatum, Vespa spp. such as V. crabro; Vespula spp such as V. squamosal; Wasmannia auropunctata, Xylocopa sp.; insects from the order Orthoptera for example Acheta domesticus, Calliptamus italicus, Chortoicetes terminifera, Ceuthophilus spp, Diastrammena asynamora, Dociostaurus maroccanus, Gryllotalpa spp such as G. africana, G. gryllotalpa; Gryllus spp, Hieroglyphus daganensis, Kraussaria angulifera, Locusta spp. such as L. migratoria, L. pardalina; Melanoplus spp such as M. bivittatus, M. femurrubrum, M. mexicanus, M. sanguinipes, M. spretus; Nomadacris septemfasciata, Oedaleus senegalensis, Scapteriscus spp, Schistocerca spp such as S. americana, S. gregaria, Stemopelmatus spp, Tachycines asynamorus, and Zonozerus variegatus; pests from the Class Arachnida for example Acari, e.g. of the families Argasidae, Ixodidae and Sarcoptidae, such as Amblyomma spp. (e.g. A. americanum, A. variegatum, A. maculatum), Argas spp. such as A. persicu), Boophilus spp. such as B. annulatus, B. decoloratus, B. microplus, Dermacentor spp such as D. silvarum, D. andersoni, D. variabilis, Hyalomma spp. such as H. truncatum, Ixodes spp. such as I. ricinus, I. rubicundus, I. scapularis, I. holocyclus, I. pacificus, Rhipicephalus sanguineus, Ornithodorus spp. such as O. moubata, O. hermsi, O. turicata), Ornithonyssus bacoti, Otobius megnini, Dermanyssus gallinae, Psoroptes spp such as P. ovis, Rhipicephalus spp such as R. sanguineus, R. appendiculatus, Rhipicephalus evertsi), Rhizoglyphus spp; Sarcoptes spp. such as S. Scabiei; and Family Eriophyidae including Aceria spp such as A. sheldoni, A. anthocoptes, Acallitus spp; Aculops spp. such as A. lycopersici, A. pelekassi; Aculus spp such as A. schlechtendali; Colomerus vitis, Epitrimerus pyri, Phyllocoptruta oleivora; Eriophytes ribis and Eriophyes spp such as Eriophyes sheldoni; Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp. such as B. phoenicis; Family Tetranychidae including Eotetranychus spp., Eutetranychus spp., Oligonychus spp., Petrobia latens, Tetranychus spp such as T. cinnabarinus, T. evansi, T. kanzawai, T, pacificus, T. phaseulus, T. telarius and T. urticae; Bryobia praetiosa; Panonychus spp. such as P. ulmi, P. citri; Metatetranychus spp. and Oligonychus spp. such as O. pratensis, O. perseae), Vasates lycopersici; Raoiella indica, Family Carpoglyphidae including Carpoglyphus spp; Penthaleidae spp such as Halotydeus destructor; Family Demodicidae with species such a Demodex spp; Family Trombicidea including Trombicula spp; Family Macronyssidae including Ornothonyssus spp; Family Pyemotidae including Pyemotes tritici; Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa; pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. schachtii, H. trifolii; Seed gall nematodes, Anguina spp.; Stem and foliar nematodes, Aphelenchoides spp. such as A. besseyi; Sting nematodes, Belonolaimus spp. such as B. longicaudatus; Pine nematodes, Bursaphelenchus spp. such as B. lignicolus, B. xylophilus; Ring nematodes, Criconema spp.; Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Heliocotylenchus multicinctus; Sheath and sheathoid nematodes, Hemicycliophora spp. and Hemicriconemoides spp.; Hirshmanniella spp.; Lance nematodes, Hoploaimus spp.; False rootknot nematodes, Nacobbus spp.; Needle nematodes, Longidorus spp. such as L. elongatus; Lesion nematodes, Pratylenchus spp. such as P. brachyurus, P. neglectus, P. penetrans, P. curvitatus, P. goodeyi; Burrowing nematodes, Radopholus spp. such as R. similis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. primitivus; Paratrichodorus spp. such as P. minor; Stunt nematodes, Tylenchorhynchus spp. such as T. claytoni, T. dubius; Citrus nematodes, Tylenchulus spp. such as T. semipenetrans; Dagger nematodes, Xiphinema spp.; and other plant parasitic nematode species; insects from the order Isoptera for example Calotermes flavicollis, Coptotermes spp such as C. formosanus, C. gestroi, C. acinaciformis; Cornitermes cumulans, Cryptotermes spp such as C. brevis, C. cavifrons; Globitermes sulfureus, Heterotermes spp such as H. aureus, H. longiceps, H. tenuis; Leucotermes flavipes, Odontotermes spp., Incisitermes spp such as I. minor, I. Snyder; Marginitermes hubbardi, Mastotermes spp such as M. darwiniensis Neocapritermes spp such as N. opacus, N. parvus; Neotermes spp, Procornitermes spp, Zootermopsis spp such as Z. angusticollis, Z. nevadensis, Reticulitermes spp. such as R. hesperus, R. tibialis, R. speratus, R. flavipes, R. grassei, R. lucifugus, R. santonensis, R. virginicus; Termes natalensis; insects from the order Blattaria for example Blatta spp such as B. orientalis, B. lateralis; Blattella spp such as B. asahinae, B. germanica; Leucophaea maderae, Panchlora nivea, Periplaneta spp such as P. americana, P. australasiae, P. brunnea, P. fuligginosa, P. japonica; Supella longipalpa, Parcoblatta pennsylvanica, Eurycotis floridana, Pycnoscelus surinamensis; insects from the order Siphonoptera for example Cediopsylla simples, Ceratophyllus spp., Ctenocephalides spp such as C. felis, C. canis, Xenopsylla cheopis, Pulex irritans, Trichodectes canis, Tunga penetrans, and Nosopsyllus fasciatus; insects from the order Thysanura for example Lepisma saccharina, Ctenolepisma urbana, and Thermobia domestica; pests from the class Chilopoda for example Geophilus spp., Scutigera spp. such as Scutigera coleoptrata; pests from the class Diplopoda for example Blaniulus guttulatus, Julus spp, Narceus spp.; pests from the class Symphyla for example Scutigerella immaculata; insects from the order Dermaptera, for example Forficula auricularia; insects from the order Collembola, for example Onychiurus spp. such as Onychiurus armatus; pests from the order Isopoda for example, Armadillidium vulgare, Oniscus asellus, Porcellio scaber; insects from the order Phthiraptera, for example Damalinia spp., Pediculus spp. such as Pediculus humanus capitis, Pediculus humanus corporis, Pediculus humanus humanus; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp. Examples of further pest species which may be controlled by the compounds and/or compositions identified herein include: from the Phylum Mollusca, class Bivalvia, for example, Dreissena spp.; class Gastropoda, for example, Anion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti.

The terms “non-pest” and/or “non-target” species refers to all other animals that would not be classified as “pests” and/or “pest species” as defined above.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides, conservatively modified variants thereof, complementary sequences, and degenerate codon substitutions that have similar binding properties as the reference nucleic acid and are metabolized in a manner similar to naturally occurring nucleotides.

The terms “nucleic acid” or “nucleic acid molecule” can also be used interchangeably with gene, open reading frame (ORF), cDNA, and mRNA encoded by a gene, nucleic acid molecule, nucleic acid fragment, nucleic acid segment, or polynucleotide.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

The term “gene delivery” or “gene transfer” refers to methods or systems for reliably introducing foreign DNA into target cells and include injection, transduction, transfection, transformation and electroporation. Such methods can result in transient or long term expression of genes.

The term “transduction” refers to the delivery of a DNA molecule to a recipient cell either in vivo or in vitro, via a replication-defective viral vector, such as, e.g., adenoviral vector.

The term “transfection” is used to refer to the uptake of foreign DNA by a mammalian cell. A cell has been “transfected” when exogenous DNA has been introduced across the cell plasma membrane. Transfection can be used to introduce one or more exogenous DNA moieties, such as a plasmid vector and other nucleic acid molecules, into suitable cells. The term refers to both stable and transient uptake of the genetic material.

The term “transformation” and/or “transfection” refers to a process for introducing heterologous DNA into a cell. Transformed cells are understood to encompass not only the end product of a transformation/transfection process, but also transgenic progeny thereof.

The term “injection” refers to a process for introducing heterologous DNA or RNA into a cell using a syringe and a needle.

The term “vector” refers to any genetic element, such as a plasmid, phage, transposon, cosmid, chromosome, virus, virion, etc., which is capable of replication when associated with the proper control elements, such as a helper virus, and which can transfer gene sequences between cells. Thus, the term includes cloning and expression vehicles, as well as replication-defective viral vectors.

The terms “expression vector” and “expression cassette” refer to a nucleic acid molecule capable of directing expression of a particular nucleotide sequence in an appropriate host cell, typically comprising a promoter operatively linked to the nucleotide sequence of interest which is operatively linked to termination signals. It also typically comprises sequences required for proper translation of the nucleotide sequence. The coding region usually encodes a polypeptide(s) of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression. Typically, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant, the promoter can also be specific to a particular tissue, organ, or stage of development.

The term “adenovirus” refers to a vector derived from an adenovirus serotype. Adenoviruses are double-stranded DNA viruses capable of infecting broad range of mammalian cells, both dividing and non-dividing. The adenovirus DNA is linear of approximately 36,000 bp. The most widely used adenoviral vectors for gene delivery are the replication-deficient vectors, in which the E1 and E3 regions of the adenoviral genome are deleted. E1-deleted viruses are propagated in complementing cells, such as HEK-293, which provide E1-encoded proteins in trans. E1/E3-deleted adenoviruses can accommodate up to 7.5 kb of foreign DNA.

The classical method of incorporating foreign DNA into adenovirus genome involves homologous recombination in mammalian cells. More efficient method, as described in the present examples involves homologous recombination in Escherichia coli between a large plasmid containing most of the adenovirus genome and a small shuttle plasmid. The shuttle plasmid contains the expression cassette flanked by sequences homologous to the region to be targeted in the viral genome. The recombinant Ad genome is then linearized by restriction digestion and used to transfect E1-complementing mammalian cells to produce viral particles (Douglas J T., Methods Mol Biol. 2004; V246:3-14) To produce adenoviruses containing toxic gene, special adenoviral vectors have been developed where expression of foreign gene is suppressed during virus production. One of these systems, named, SpeAd Ad-teto system is offered by, for example, Welgen, Inc.

The term “tetracycline-controlled transactivator” (tTA) refers to a fusion protein used to control nucleic acid expression in the presence or absence of doxyclycline, tetracycline and related compounds. The tTA includes a Tet repressor (TetR) fused to any domain capable of activating transcription. The tTA may include a TetR fused to a C-terminal portion of adenovirus.

The term “operably linked” refers to two nucleic acid sequences that are related physically or functionally. For example, a promoter or regulatory DNA sequence is said to be “operably linked to” a DNA sequence that encodes an RNA or a polypeptide if the two sequences are situated such that the regulatory DNA sequence will affect the expression level of the coding or structural DNA sequence. A promoter is also said to be operably linked to a nucleotide sequence if when an RNA polymerase binds to the promoter under conditions sufficient for transcription, the nucleotide sequence is transcribed.

The term “isolated,” when applied to a nucleic acid or polypeptide, denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high performance liquid chromatography. A polypeptide that is the predominant species present in a preparation is substantially isolated. The term “isolated” denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 99% pure.

The terms “label” and “labeled” refer to the attachment of a moiety, capable of detection by spectroscopic, radiologic, or other methods, to a molecule. Thus, the terms “label” or “labeled” refer to incorporation or attachment, optionally covalently or non-covalently, of a detectable marker into a molecule, such as a biomolecule. Various methods of labeling biomolecules are known in the art and can be used. Examples of labels for biomolecules include, but are not limited to, the following: radioisotopes, fluorescent labels, heavy atoms, enzymatic labels or reporter genes, chemiluminescent groups, and biotinyl groups. In some embodiments, labels are attached by spacer arms of various lengths to reduce potential steric hindrance. Fluorescent probe that can be utilized include, but are not limited to fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3, 3.5, 5, 5.5, and 7; phycoerythrin; phycoerythrin-Cy conjugates; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives (e.g., hydroxycoumarin, aminocoumarin, and methoxycoumarin); pyrenes; pyridyloxazole derivatives; dapoxyl dyes; Cascade Blue and Yellow dyes; benzofuran isothiocyanates; sodium tetrafluorophenols; 4,4-difluoro-4-bora-3a,4a-diaza-s-indacene; Alexa fluors (e.g., 350, 430, 488, 532, 546, 555, 568, 594, 633, 647, 660, 680, 700, and 750); isotope of hydrogen (e.g., tritium isotope, 3H), isotope of carbon (e.g., 14C) green fluorescent protein; yellow fluorescent protein or red fluorescent protein. The peak excitation and emission wavelengths will vary for these compounds and selection of a particular fluorescent probe for a particular application can be made in part based on excitation and/or emission wavelengths.

As used herein, the terms “candidate compound” or “test compound” refer to the collection of compounds that are to be screened for their ability to bind to Nanchung proteins, Nanchung and TRP protein complexes, Nanchung and Iav protein complexes, and/or modulate insect TRPV channels and/or insect TRP channels expressing a Nanchung protein. The candidate compounds may encompass numerous classes of chemical molecules, e.g., small organic or inorganic molecules, polysaccharides, biological macromolecules, e.g., peptides, proteins, peptide analogs and derivatives, peptidomimetics, nucleic acids, nucleic acid analogs and derivatives, an extract made from biological materials such as bacteria, plants, fungi, or animal cells or tissues, naturally occurring or synthetic compositions, or combinations thereof. Generally, the candidate compounds can have a molecular weight of about 50 to 500,000, but is not limited thereto.

As used herein, the term “small molecule” refers to a compound that is “natural product-like,” however, the term “small molecule” is not limited to a “natural product-like” compound. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

In some embodiments, the candidate compound may be a synthetic molecule. The term “synthetic molecule” refers to a molecule that does not occur in nature.

In certain embodiments, the candidate compound may be a naturally-occurring molecule. Such a naturally-occurring molecule may be used in a purified or unpurified form, i.e., as obtained from the biological source. The term “naturally occurring” refers to an entity (e.g., a cell, biomolecule, etc.) that is found in nature as distinct from being artificially produced by man. For example, a polypeptide or nucleotide sequence that is present in an organism in its natural state, which has not been intentionally modified or isolated by man in the laboratory, is naturally occurring. As such, a polypeptide or nucleotide sequence is considered “non-naturally occurring” if it is encoded by or present within a recombinant molecule, even if the amino acid or nucleic acid sequence is identical to an amino acid or nucleic acid sequence found in nature.

Depending upon the particular embodiment being practiced, the candidate compounds may be provided free in solution, or may be attached to a carrier, or a solid support, e.g., beads. A number of suitable solid supports may be employed for immobilization of the candidate compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, the candidate compounds may be screened individually, or in groups. Group screening is particularly useful where hit rates for effective candidate compounds are expected to be low such that one would not expect more than one positive result for a given group.

There are millions of possible candidate compounds. Methods for developing small molecule, polymeric and genome based libraries are known and described, for example, in Ding, et al. J. Am. Chem. Soc. 124: 1594-1596 (2002) and Lynn, et al., J. Am. Chem. Soc. 123: 8155-8156 (2001). A number of small molecule libraries are known in the art and commercially available. Commercially available compound libraries can be obtained from, e.g., ArQule, Pharmacopia, graffinity, Panvera, Vitas-M Lab, Biomol International and Oxford. These libraries can be screened using the screening methods described herein. Chemical compound libraries such as those from of 10,000 compounds and 86,000 compounds from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

The term “modulate” refers to an increase, decrease, or other alteration of any, or all, chemical and/or biological activities and/or properties of a biomolecule, such as the TRPV (e.g., insect TRPV) channel of the present invention. The term “modulation” as used herein thus refers to both upregulation (i.e., activation or stimulation) and downregulation (i.e., inhibition or suppression) of such an activity or property. As would be understood by one of ordinary skill in the art, a modulation of a chemical and/or biological activity and/or property of a biomolecule, such as TRPV channel, can result from an increase or decrease in the expression of the biomolecule in a cell. Accordingly, the terms “modulate” and grammatical variants thereof are intended to encompass both direct modulation (e.g., inhibition of a chemical and/or biological activity and/or property of a polypeptide via binding of an inhibitor to the polypeptide) as well as indirect modulation (e.g., upregulation or downregulation of expression of a protein, such as a TRPV channel or inhibition or stimulation of a biomolecule that acts together with a biomolecule of the presently disclosed subject matter to produce a biological effect).

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term TRP channel refers to a channel and/or a protein complex formed from one or more of the following: Nanchung proteins, TRP proteins and/or Inactive protein. It is to be understood that the TRPV channel and/or TRPV protein complex is a type of TRP channel and/or TRP protein complex. Nanchung proteins and Inactive proteins are a type of TRPV proteins which are a type of TRP proteins. TRP proteins may be found in insect cellular membranes; mammalian cellular membranes; derived from a free cell system; produced by a cell culture; and/or produced by cells, including but not limited to a frog egg, insect cell, and/or mammalian cell, that have been genetically modified (including either or both DNA and/or RNA) to include the genes coding for one or more of the Nanchung proteins, TRP proteins and/or the Inactive proteins. It is to be understood that the TRP proteins disclosed herein may be mammalian TRP proteins and/or arthropod TRP proteins and/or Nematode TRP proteins and/or Mollusk TRP proteins. It is to be further understood that Nanchung and Inactive proteins may be found in arthropods, including, but not limited to insects, as well as Mollusks and/or Nematodes. It is to be understood that TRP and/or TRPV channels may be formed from one or more TRP and/or TRPV protein complexes; from two or more TRP and/or TRPV protein complexes; from three or more TRP and/or TRPV protein complexes, and/or from four or more TRP and/or TRPV protein complexes.

The term “binding affinity” refers to the ability of compounds to form a bond with a receptor. For purposes of this invention, an equilibrium constant is used to determine the binding affinity of one or more compounds with the one or more Nanchung proteins, TRP channels and or TRPV channels. A dissociation constant (Kd) is a specific type of equilibrium constant measured in a labeled compound saturation binding assay. It is the molar concentration of labeled compound that binds to half the receptor sites at equilibrium. The lower the dissociation constant the higher the binding affinity of the candidate compound for the Nanchung protein, a Nanchung TRP protein complex, and or a Nanchung Inactive protein complex. An inhibition constant (Ki) is a specific type of equilibrium constant that is measured using competition binding of an unlabeled compound with a labeled compound. It is the molar concentration of the competing unlabeled compound that would occupy 50% of the receptors at equilibrium, if no labeled compound was present. It can be calculated using the Cheng and Prusoff equation: Kᵢ=IC₅₀/(1+[L]/K_{d}) where IC₅₀ is the molar concentration of competing unlabeled compound which reduces the specific binding of a labeled compound by 50%. In this equation [L] is the molar concentration of a labeled compound in the assay; K_{d} is equilibrium dissociation constant for a labeled compound. In addition to saturation binding assay, equilibrium dissociation constant, Kd can be measured in multiple ways, including but not limited to:
1. Using the association and dissociation rates constants where Kd=Koff/Kon
2. Using a fluorescence polarization assay
3. Using surface plasmon resonance
4. Using isothermal calorimetry
5. Using Schild analysis

Surface plasmon resonance and isothermal calorimetry do not require labeled compound to measure K_{d}. In addition, for the competition assay, Kᵢ and/or K_{d} (for competition assay) may be calculated using one or more of the following: Cheng and Prusoff equation, fluorescence polarization, and/or Schild analysis. It is to be understood that the K_{d} and Kᵢ values calculated for a compound should be similar, but due to experimental error the values may have less than a two-fold difference between the two values or less than a three-fold difference between the two-values depending upon the extent of the experimental error.

The term “species specific binding” for purposes of this application refers to preferential binding of a compound to insect TRP and/or TRPV proteins versus vertebrate TRP and/or TRPV proteins.

The term “high affinity” and/or “high binding affinity” refers to a compound whose equilibrium dissociation constant (K_{d}) and/or equilibrium inhibition constant (Kᵢ) is calculated as being less than or equal to 3×10⁻⁷ M, less than or equal to 3×10⁻⁸ M, and/or less than or equal to 30×10⁻¹² M.

The lower the K_{d} or Kᵢ the higher the binding affinity for the compound.

The term “mode of action” refers to the classification of a compound and/or a composition according to the Insecticide Resistance Action Committee (IRAC) into the appropriate group based on the compound and/or composition (a) exhibiting the same function as other insecticides in that group and (b) binding directly to the same site on particular proteins, protein complexes and/or protein channels as other compounds and/or compositions in that group.

### III. Screening Methods

The inventors have discovered that insect TRP and/or TRPV channels may be used to identify compounds that block insect feeding behavior and thus be useful as insecticides. As such, in one embodiment, the present invention relates to a method for determining whether or not a candidate compound is a modulator of an insect TRP and/or TRPV channel. It is to be understood herein that insect TRP channels comprise one or more Nanchung proteins in a complex with one or more TRP proteins such that they form an ion channel. It is to be further understood that insect TRPV channels comprise one or more Nanchung proteins in a complex with one or more TRPV proteins, including but not limited to Inactive proteins, such that they form an ion channel. The inventors have further discovered methods of identifying compounds that bind directly to one or more Nanchung proteins, TRP protein complexes and/or TRPV protein complexes. Furthermore the inventors have determined that a compound, afidopyropen, binds directly to one or more Nanchung proteins, TRP and/or TRPV protein complexes and/or channels and that acts as a modulator of one or more TRP and/or TRPV protein channels. It is to be understood that Arthropod, Mollusk and/or Nematode TRP and/or TRPV channels comprise one or more Nanchung proteins in a complex with one or more TRP and/or TRPV proteins such that they form an ion channel.

The methods identified herein includes providing one or more Nanchung proteins, TRP and/or TRPV proteins, protein complexes and/or channels; contacting the first cell with a candidate compound; and assaying for modulation of the insect TRP and/or TRPV channel, wherein the modulation of the insect TRP and/or TRPV channel identifies the candidate compound as a modulator of the insect TRP and/or TRPV channel. Modulation of the TRP and/or TRPV channel may be assayed using conventional in vitro and in vivo methods well known to the skilled artisan. For example, in certain embodiments, the assaying step may include (1) detecting calcium ion mobilization in the first cell in response to the candidate compound, (2) detecting a membrane potential in the first cell in response to the candidate compound, (3) comparing calcium ion mobilization in the first cell in the absence of the candidate compound with calcium ion mobilization in the first cell in the presence of the candidate compound. For example, calcium response may be measured by assessment of the uptake of Ca²⁺ or by using fluorescent dyes, such as Fluo-4 or Fura-2 or fluorescent proteins, such as Cameleon, (4) comparing a membrane potential in the first cell in the absence of the candidate compound with a membrane potential in the first cell in the presence of the candidate compound, (5) comparing calcium ion mobilization in the first cell in the presence of the candidate compound with calcium ion mobilization reference level indicative of no modulation of the TRPV channel, and/or (6) comparing a membrane potential in the first cell in the presence of the candidate compound with a membrane potential reference level indicative of no modulation of the TRPV channel, (7) comparing ion currents in the first cell in the absence of the candidate compound with a ion currents in the first cell in the presence of the candidate compound using standard electrophysiology methods, such as voltage clamp or patch clamp. It is to be understood that this same assay could be run on TRP and/or TRPV channels from vertebrate, arthropod, mollusks, nematodes or any other organisms. Such a test would allow one to determine whether or not the candidate compound worked as a modulator is those systems and thus, help one determine the potential environmental impact of using the candidate compound as an insecticide.

Preferably, the candidate compound may modulate the calcium ion mobilization or the membrane potential, or ion currents in the first cell by at least 10% relative to the reference level; more preferably at least 20% relative to the reference level; more preferably at least 30% relative to the reference level; more preferably at least 40% relative to the reference level; more preferably at least 50% relative to the reference level; more preferably at least 60% relative to the reference level; more preferably at least 70% relative to the reference level; more preferably at least 75% relative to the reference level; more preferably at least 80% relative to the reference level; more preferably at least 85% relative to the reference level; more preferably at least 90% relative to the reference level; and more preferably at least 95% relative to the reference level.

In addition, activation of TRPV channel may be assayed using a variety of other conventional assays that measure changes in ion fluxes including, but not limited to, patch clamp techniques, measurement of whole cell currents, radiolabeled ion flux assays, and fluorescence assays using voltage sensitive dyes (Zheng at al., 2004).

Modulation of an insect TRPV channel may also be assessed using a variety of other in vitro and in vivo assays to determine functional, chemical, and physical effects, e.g., measuring the binding of the insect TRPV to other molecules, including peptides, small organic molecules, and lipids; measuring insect TRPV protein and/or mRNA levels, or measuring other aspects of insect TRPV polypeptides, e.g., transcription levels, or physiological changes.

The inventors have further determined that it is beneficial to identify compounds that bind directly to one or more Nanchung proteins and/or TRP and/or TRPV protein complexes and/or channels and thus be useful as insecticides. As such, in one embodiment, the present invention relates to a method for determining whether or not a candidate compound directly binds in a species specific manner to one or more Nanchung proteins and/or TRP and/or TRPV protein complexes and/or channels. There are two variations of binding assays, a saturation assay and a competition assay. The saturation assay may include at least one of the following steps: (1) contacting the one or more Nanchung proteins, TRP, and/or TRPV protein complexes and/or channels with a compound, wherein the compound may be labeled or unlabeled; (2) measuring the concentration of the labeled compound and/or unlabeled compound which is bound to the one or more Nanchung proteins, TRP, and/or TRPV protein complexes and/or channels; (3) calculating the equilibrium disassociation constant (K_{d}) for the one or more labeled compounds and/or unlabeled compound (4) wherein the K_{d} may be determined using a (a) maximum binding (Bₘₐₓ) and (b) the concentration of the compound needed to reach 50% of maximum binding.

The competition assay may include one or more of the following steps: (1) contacting the one or more Nanchung proteins, TRP, and/or TRPV proteins, protein complexes and/or channels with a compound, wherein the compound may be labeled or unlabeled; (2) measuring the concentration of the compound which is bound to the one or more Nanchung proteins, TRP, and/or TRPV proteins, protein complexes and/or channels; (3) calculating the equilibrium disassociation constant (K_{d}) for the one or more compounds; (4) wherein the K_{d} may be determined using one or more of the following methods: (I)(a) maximum binding (Bₘₐₓ) and (b) the concentration of labeled compound needed to reach 50% of maximum binding, (II) fluorescence polarization assay, (III) surface plasmon resonance, (IV) isothermal calorimetry, (V) Schild analysis; (5) contacting the one or more Nanchung proteins, TRP, and/or TRPV proteins, protein complexes and/or channels with a labeled compound having high affinity to the one or more Nanchung proteins, TRP, and/or TRPV proteins, protein complexes and/or channels; (6) contacting the one or more one or more Nanchung proteins, TRP, and/or TRPV proteins, protein complexes and/or channels with a candidate compound in the presence of the labeled compound of step 1; (7) increasing the amount of the candidate compound available to contact the one or more Nanchung proteins, TRP, and/or TRPV proteins, protein complexes and/or channels until binding of the labeled compound is near 0%; (8) calculating the equilibrium inhibition constant (K) and/or the equilibrium dissociation constant (K_{d}) for competition assay using one or more of the following methods: (I) (a) determining amount of bound labeled compound in the absence of the candidate compound and (b) determining concentration of the candidate compound that is required to reduce the amount of bound labeled compound by 50%, (II) fluorescent polarization assay, and/or (III) Schild analysis; (9) comparing the inhibition constant of the candidate compounds to other candidate compounds; (10) comparing the inhibition constant of the candidate compounds to the disassociation constant of the labeled compound.

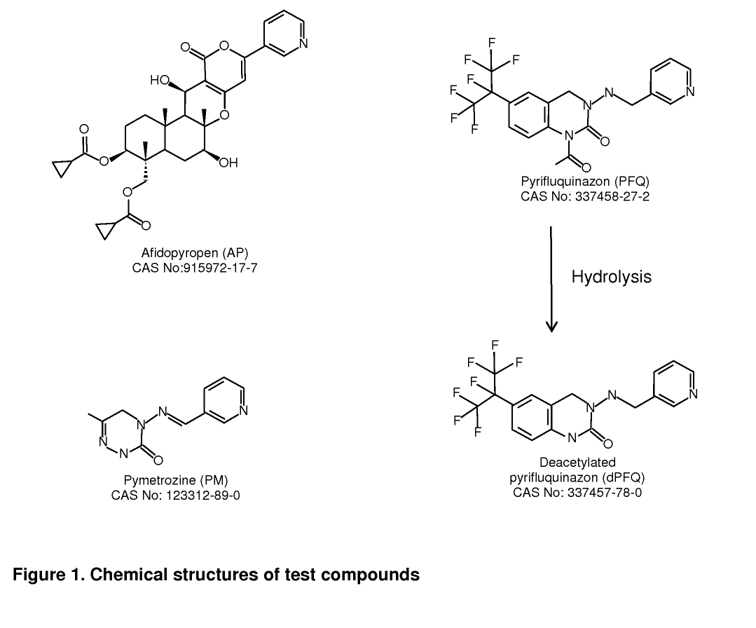

Binding to a one or more Nanchung proteins, TRP protein complexes and/or channels and/or TRPV protein complexes and/or channels may be assessed using a variety of in vitro and in vivo assays to determine whether the candidate compound binds directly to the one or more Nanchung proteins, TRP protein complexes and/or channels and/or TRPV protein complexes and/or channels. Identification of molecular targets of insecticides is essential for assessing their spectra, health and environmental impacts, as well as for insect resistance management. Pymetrozine (PM) and pyrifluquinazon (PFQ) are two structurally-related commercial insecticides (chemical structures are shown on FIG. 1) which act by disrupting coordination and feeding of plant-sucking insects such as aphids and whiteflies (Casida and Durkin, 2013; Maienfisch, 2012). Pyrifluquinazon can spontaneously de-acetylate in aqueous solutions. The de-acetylated form of pyrifluquinazon (dPFQ) was shown to be significantly more potent than the parent compound, suggesting that pyrifluquinazon is a pro-drug, which is activated through de-acetylation (Nesterov et al., 2015). dPFQ was, therefore, used in the present invention, however it is to be understood that pyrifluquinazon may be used as well.

Afidopyropen is a new insecticide compound with a previously unknown mode of action. Although afidopyropen and Pymetrozine have significantly different chemical structures (FIG. 1), they have similar spectrum of insects they are toxic to, and produce similar behavioral symptoms, including feeding inhibition in aphids (Leichter et al., 2013). However, whether afidopyropen and Pymetrozine bind to the same location and thus would be classified as having the same mode of action was unknown.

TRP and/or TRPV (Transient Receptor Potential) proteins form hetero- and homotetrameric cation channels in various cell types (Venkatachalam and Montell, 2007). Pymetrozine and pyrifluquinazon disturbed Drosophila coordination and hearing by overstimulating, and ultimately silencing TRPV channels in chordotonal organs (Nesterov et al., 2015), which are serially arranged stretch receptors (Kavlie and Albert, 2013) which sense relative position of body parts in insects (Kamikouchi et al., 2009; Sun et al., 2009). While Pymetrozine and pyrifluquinazon were found to bind to the Nanchung protein alone, the modulation effect of the Pymetrozine and pyrifluquinazon required simultaneous presence of two TRPV proteins, Nanchung (Nan) and Inactive (Iav), which co-occur in chordotonal neurons and form functional ion channels. (Nesterov et al., 2015, PCT/US2014/071489, which is hereby incorporated by reference in its entirety.)

The current invention shows that afidopyropen, Pymetrozine and de-acetylated pyrifluquinazon, modulate TRP channels directly, by directly binding to the same site on these protein channels. Furthermore, the current invention identifies methods of determining whether or not a candidate compound binds directly to the same site on these proteins, protein complexes and/or protein channels. Determining the binding location and affinities for candidate compounds assists with the classification of a compounds mode of action and allows for better insect management and control that may result in decreased risk of developing resistant insect populations.

The current invention also found that there was increased binding affinity of Pymetrozine, de-acetylated pyrifluquinazon, and afidopyropen in the presence of both the Nanchung protein and the Inactive protein. The current invention also found that afidopyropen exhibited modulation activity only in the presence of both Nanchung and Inactive proteins, protein complexes and/or channels. The current invention also found that afidopyropen exhibited modulation activity in the presence of both Nanchung and TRP proteins, protein complexes and/or channels. Furthermore the current invention identified afidopyropen as having a high binding affinity to the Nanchung protein, a Nanchung-TRP protein complex, a Nanchung-TRP protein channel, a Nanchung-TRPV protein complex, Nanchung-TRPV protein channel, a Nanchung-Inactive protein complex, and a Nanchung-Inactive protein channel. Accordingly, the current invention identifies a labeled compound having a high binding affinity to one or more Nanchung proteins, TRP and/or TRPV proteins, protein complexes and/or protein channels, such as afidopyropen, pymetrozine and/or de-acetylated pyrifluquinazon, may be used to calculate the binding affinity of a candidate compound using the competition assay as described herein.

As noted previously, the candidate compound may be a small organic molecule, small inorganic molecule, polysaccharides, peptides, proteins, nucleic acids, an extract made from biological materials such as bacteria, plants, fungi, animal cells, animal tissues, and any combination thereof.

Generally, a candidate compound can be tested at any concentration that can modulate the activity of a TRP and/or TRPV channel, and/or exhibit binding to TRP and/or TRPV proteins. In some embodiments, the candidate compound may be tested at a concentration in the range of about 0.01 nM to about 1000 mM. In certain other embodiments, the compound may be tested in the range of about 100 μM to about 1000 μM. In certain further embodiments, the candidate compound may be tested at greater than 0.05 mM, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.0 mM, or 2 mM. Other ranges and concentrations of the candidate compound may also be suitable.

In some embodiments, the candidate compound may be tested at two or more different concentrations. Preferably the highest concentration tested is at least 2×, at least 3×, at least 4×, at least 5×, at least 6×, at least 7×, at least 8×, at least 9×, at least 10×, at least 15×, at least 20×, at least 25×, at least 50×, at least 75×, at least 100×, at least 200×, at least 250× higher than the lowest concentration employed. For example, the candidate compound may be tested at 0.1 mM, 0.5 mM, and 1 mM, where the highest concentration is at least 10× the concentration of the lowest concentration employed.

Generally, the one or more Nanchung proteins, TRP and/or TRPV protein complexes and/or channels may be contacted with a candidate compound for any suitable length of time before measuring the binding affinity and/or activity of the insect TRP and/or TRPV channel and/or proteins. For example, the insect TRPV channel may be contacted with a candidate compound for at least 5 seconds, at least 10 seconds, at least 15 seconds, at least 30 seconds, at least 45 seconds, at least 1 minute, at least 2 minutes, at least 3 minutes, at least 4 minutes, at least 5 minutes, at least 10 minutes, at least 15 minutes, at least 30 minutes, at least 60 minutes, at least 2 hours, at least 3 hours or more before activity and/or binding of the insect TRP and/or TRPV is measured. In some embodiments, activity may be measured at the instant when the insect TRPV is contacted with a candidate compound.

In some embodiments, activity of insect TRPV channel may be measured over a period of time as well as binding of the candidate compound to the one or more Nanchung proteins, TRP and/or TRPV protein complexes and/or channels. For example, activity may be measured for a period of at least 5 seconds, at least 10 seconds, at least 15 seconds, at least 30 seconds, at least 45 seconds, at least 1 minute, at least 2 minutes, at least 3 minutes, at least 4 minutes, at least 5 minutes, at least 10 minutes, at least 15 minutes, at least 30 minutes, at least 60 minutes, at least 2 hours, at least 3 hours, at least 4 hours or more. The measurement period can start before the one or more Nanchung proteins, TRP and/or TRPV protein complexes and/or channels is contacted with a candidate compound, at the instant when the one or more Nanchung proteins, TRP and/or TRPV protein complexes and/or channels is first contacted with a candidate compound or start after a period of time after the one or more Nanchung proteins, TRP and/or TRPV protein complexes and/or channels is first contacted with a candidate compound. The one or more Nanchung proteins, TRP and/or TRPV protein complexes and/or channels may be continuously contacted with the candidate compound while activity and/or binding is measured.

In some embodiments, the candidate compound has an effective concentration EC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM for activating and/or binding an insect-specific TRPV channel.

In some embodiments, the candidate compound has an IC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM for inhibiting and/or binding an insect-specific TRPV channel.

In certain embodiments, the candidate compound is a modulator that inhibits the activity of the TRP and/or TRPV channel.

In certain other embodiments, the candidate compound is a modulator that activates the TRP and/or TRPV channel of insects and/or other pests.

Preferably, the candidate compound is a modulator that inhibits an insect feeding behavior.

In certain embodiments the candidate compound binds directly to the Nanchung protein

In certain other embodiments the candidate compound binds directly to a complex consisting of the one or more Nanchung proteins and one or more TRP proteins.

Preferably the candidate compound binds directly to the one or more Nanchung and Inactive protein complexes.

In certain embodiments, to determine whether a candidate compound preferentially modulates and/or binds to the insect-specific TRPV and/or TRP channel relative to a mammalian-specific TRPV and/or TRP channel, the method may further include providing a second cell expressing a mammalian TRPV and/or TRP channel. The second cell may be contacted with the candidate compound and modulation of and/or binding to the mammalian TRPV and/or TRP channel may be assayed by the methods known in the art, which were also described herein. Following the assaying steps the modulation of and/or binding to the insect TRPV and/or TRP channel may be compared with the modulation of and/or binding to the mammalian TRPV and/or TRP channel and the candidate compound that displays an increased modulation of and/or binding to the insect-specific TRPV and/or TRP channel relative to the mammalian TRPV and/or TRP channel identifies the candidate compound as a selective modulator of and/or binder of the insect TRPV and/or TRP channel. It is to be understood that this method may be employed to determine preferential binding between other species and/or binding to pest species and not to non-pest species as well.

By the term “preferential modulation” is meant that activity or other property of insect-specific TRPV and/or TRP channel is modulated by at least 5%, at least 10%, at least 20%, at least 30%, at least 40%, at least 50%, at least 60%, at least 70%, at least 80%, at least 90%, at least 95%, at least 100%, or more relative to a reference level or the mammalian-specific TRPV and/or TRP channel.

By the term “preferential binding” is meant that the equilibrium binding constant (K_{d} or Kᵢ) of the insect-specific TRPV and/or TRP channel is decreased by at least 5%, at least 10%, at least 20%, at least 30%, at least 40%, at least 50%, at least 60%, at least 70%, at least 80%, at least 90%, at least 95%, at least 100%, or more relative to a reference level or the mammalian-specific TRPV and/or TRP channel.

In certain other embodiments, non-selective modulators and/or binders of the TRPV and/or TRP channel may be identified. The term “non-selective” in connection with a modulator and/or binder of the TRPV and/or TRP proteins, protein complexes and/or channels means that a compound can modulate and/or bind directly to one or more TRPV and/or TRP proteins, protein complexes and/or channels in different organisms, e.g., a compound may modulate both, arthropod, such as insect, and mammalian TRPV and/or TRP protein complexes and/or channels.

In certain other embodiments, the candidate compound is an insect-specific TRP channel modulator and/or binder and does not modulate the activity of and/or bind to the mammalian-specific TRP, e.g., the tested compound has no significant effect on the mammalian-specific TRPV channel.

In certain embodiments, the one or more Nanchung protein, TRPV and/or TRP protein complex and/or channel is in a biological cell. In some embodiments, the mammalian TRPV and/or TRP channel is in a biological cell. The term “biological cell” or “cell” as used herein has its commonly understood meaning. Inside a cell, the Nanchung protein, TRPV and/or TRP channel may be expressed from an endogenous gene in the cell or from at least one vector that is transfected into the cell. It is to be understood that either the cultured cell, or a cell obtained directly from animal, such as frog oocyte, or whole tissue may be used as a source of the Nanchung, TRP and/or TRPV proteins and/or channels. Preferably, the TRPV channel proteins, Nanchung and Inactive proteins are co-expressed in the cell. The Nanchung and Inactive proteins may be co-expressed at varying rations, preferably, the ratio of the Nanchung to Inactive proteins co-expressed in the first cell is about 3:1 to about 1:3, more preferably about 1:1. It is to be understood that the Nanchung protein may be combined with other TRP channel proteins and that binding to the Nanchung protein in this scenario will have similar effects for purposes of the candidate compound acting as an insecticide. The Nanchung and TRP proteins may be co-expressed at varying rations, preferably, the ratio of the Nanchung to TRP proteins co-expressed in the first cell is about 3:1 to about 1:3, more preferably about 1:1.

In certain embodiments, the one or more Nanchung proteins TRPV and/or TRP protein complexes and/or channels are formed in a cell-free system. The term “cell-free system” as used herein means an in vitro system used to study biological reactions that happen within cells while reducing the complex interactions found in a whole cell. Subcellular fractions can be isolated by ultracentrifugation to provide molecular machinery that can be used in reactions in the absence of many of the other cellular components. Cell-free biosystems can be prepared by mixing one or more of the following: ribosomes; and/or DNA and/or RNA encoding for one or more proteins of interest; membranes; as well as a one or more enzymes and/or coenzymes. Cell-free biosystems have several advantages suitable in industrial applications. In vitro biosystems can be easily controlled. Cell-free systems provide an alternative choice for fast protein synthesis and very high product yields are usually accomplished without the formation of by-products or synthesis of cell mass.

In certain embodiments a cell genetically modified to contain the genes coding for one or more of the proteins Nanchung, TRP and/or Inactive may be used to produce the TRP and/or TRPV channel complex for purposes of the methods described herein.

In certain embodiments a nucleic acid coding for one or more of the proteins Nanchung, TRP and/or Inactive may be introduced into frog oocytes or other cells (parental cells) to produce the TRP and/or TRPV channel complex for purposes of the methods described herein.

The insect TRPV and/or TRP protein complex and/or channel used in the screening assay of the present invention may be any insect TRPV and/or TRP protein complex and/or channel or homolog or a conservative variant thereof. For example, the TRPV and/or TRP protein complex and/or channel may be a Drosophila TRPV and/or other member of TRP channel superfamily. Other sources of TRPV and/or TRP protein complex and/or channel may also be suitable for use according to the methods of the present invention.

Insect TRP and/or TRPV channels are thought to be associated with one or more Nanchung proteins and may also include one or more Inactive proteins. The mammalian TRP and/or TRPV channels used in the screening assay may be any mammalian TRP or homolog thereof, preferably a TRPV channel similar to an insect TRPV channel.

In some embodiments, the Nanchung nucleic acid sequences may be selected from the group consisting of a) a polynucleotide molecule comprising a nucleic acid molecule having a sequence selected from the group consisting of SEQ ID NOS: 1-27 (FIGS. 10-36); b) a polynucleotide molecule having at least about 70% sequence identity to the sequence of SEQ ID NOS: 1-27 (FIGS. 10-36); and c) a fragment of the polynucleotide molecule of a) or b). Such fragments can be a UTR, a core promoter, an intron, an enhancer, a cis-element, or any other regulatory element.

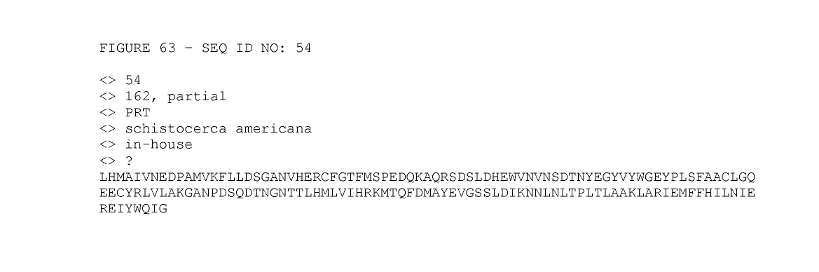

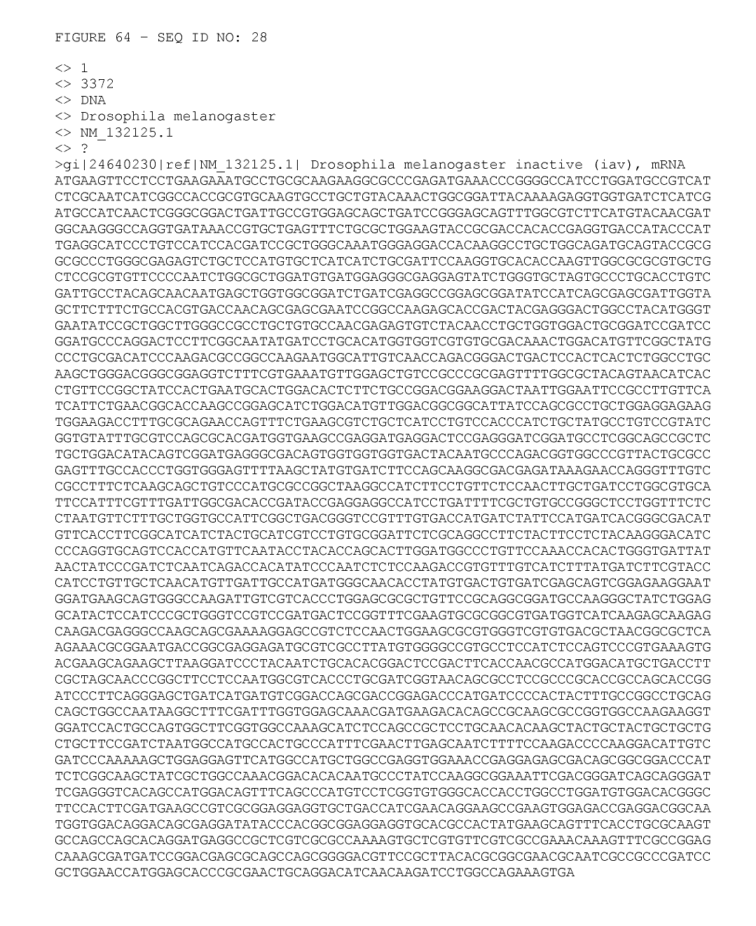

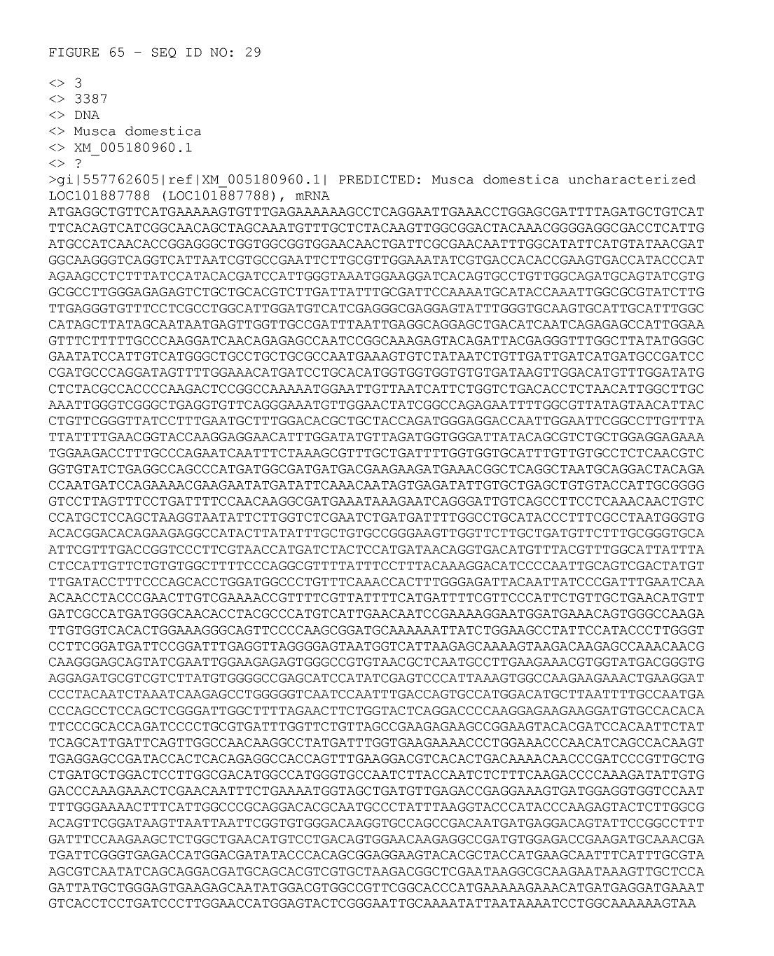

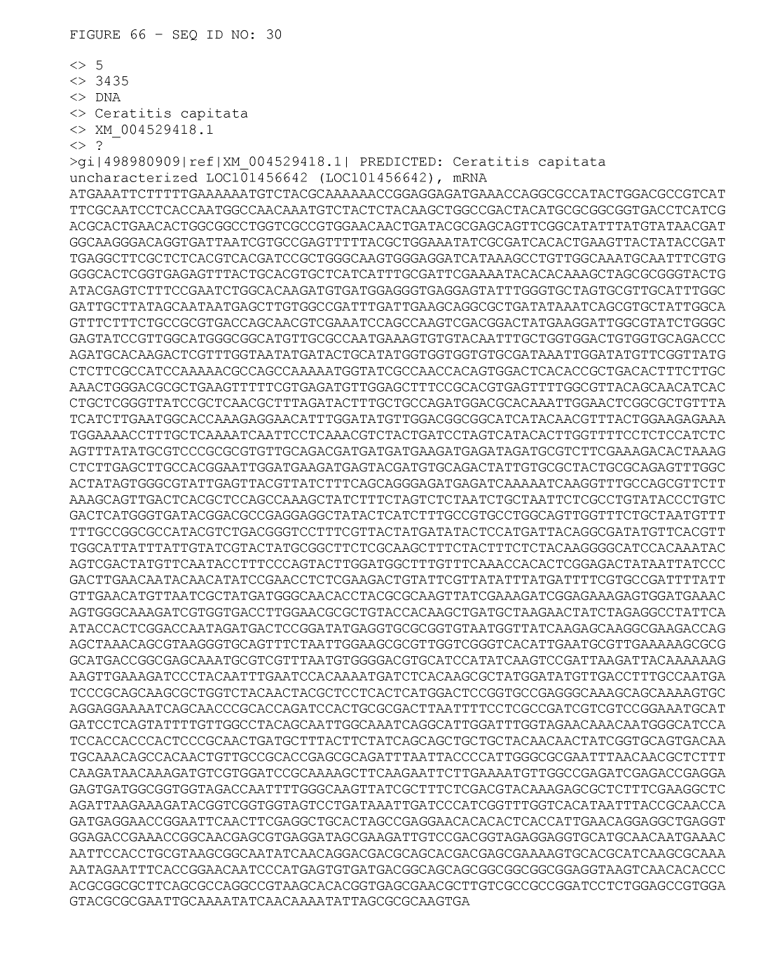

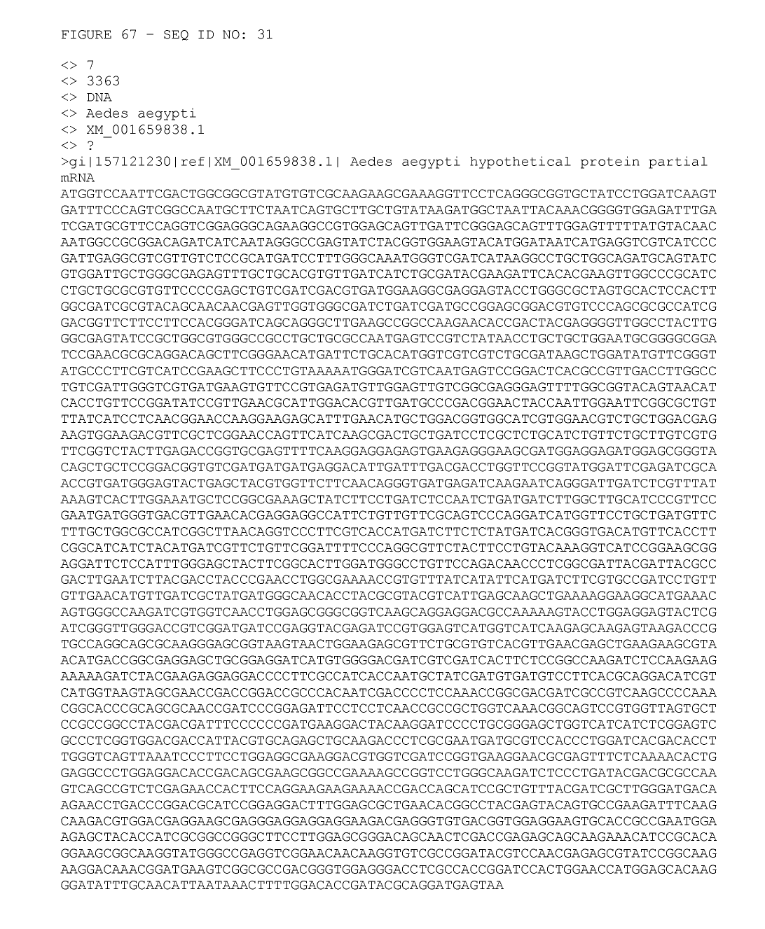

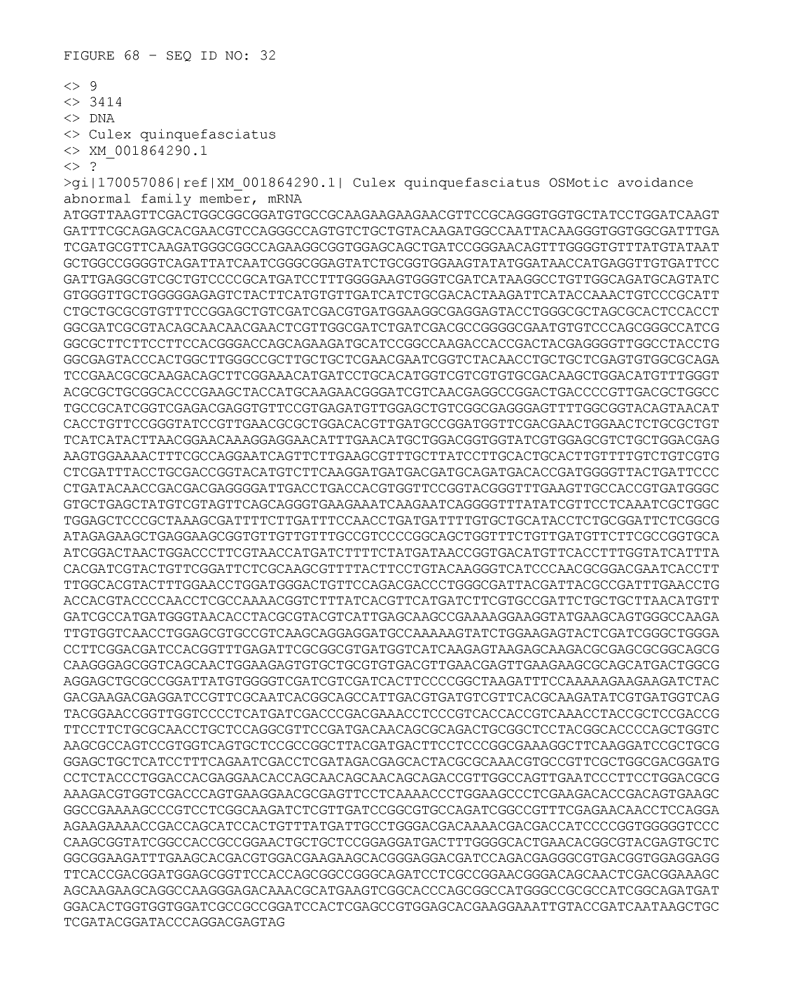

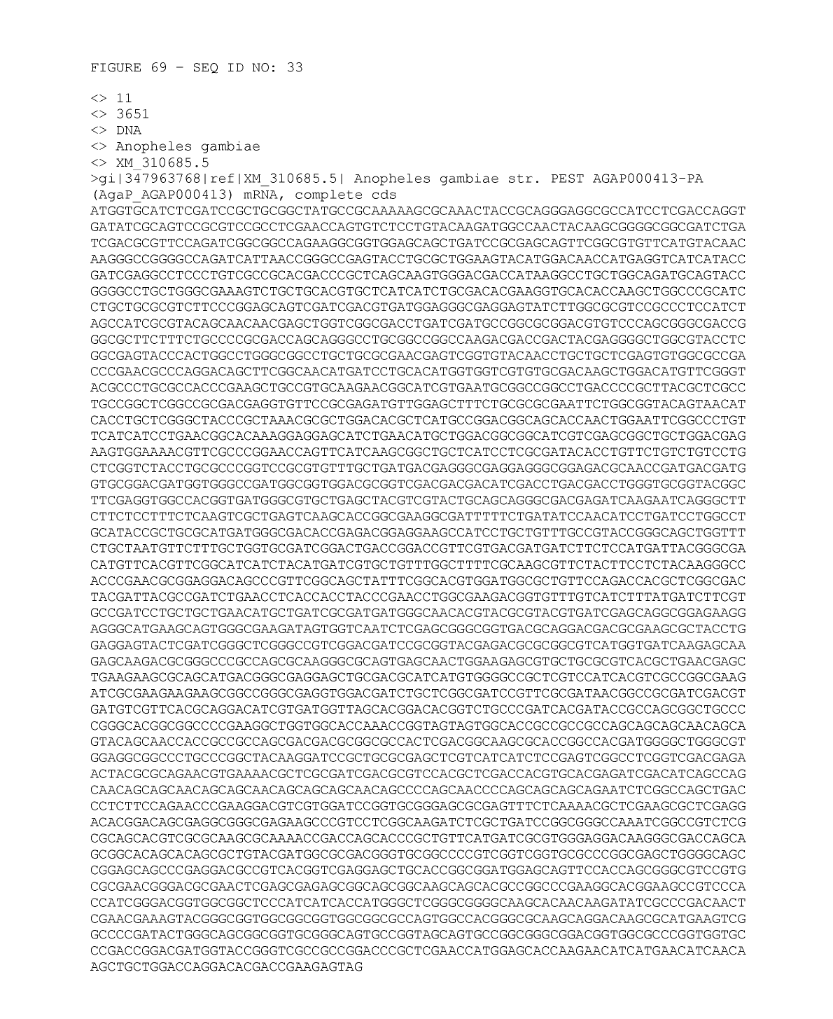

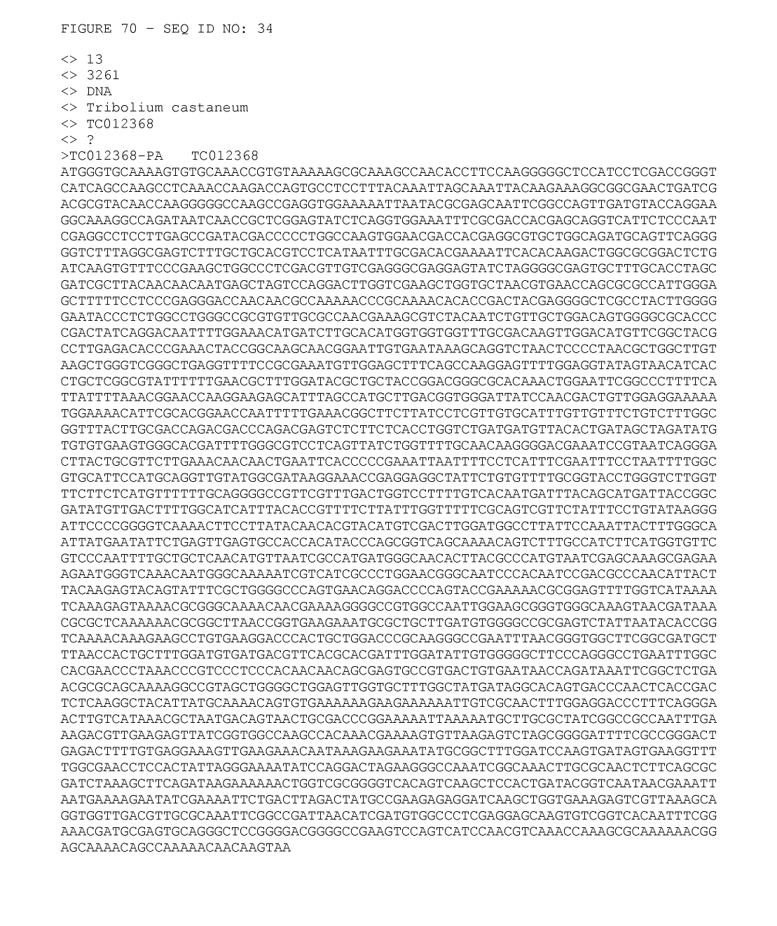

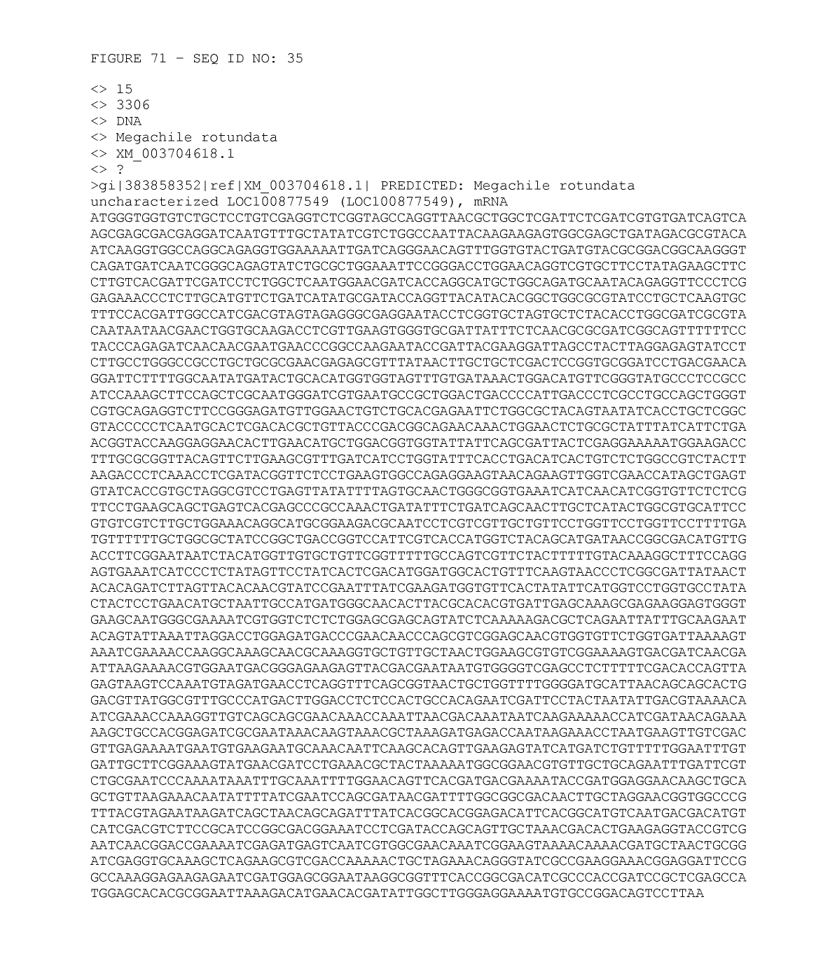

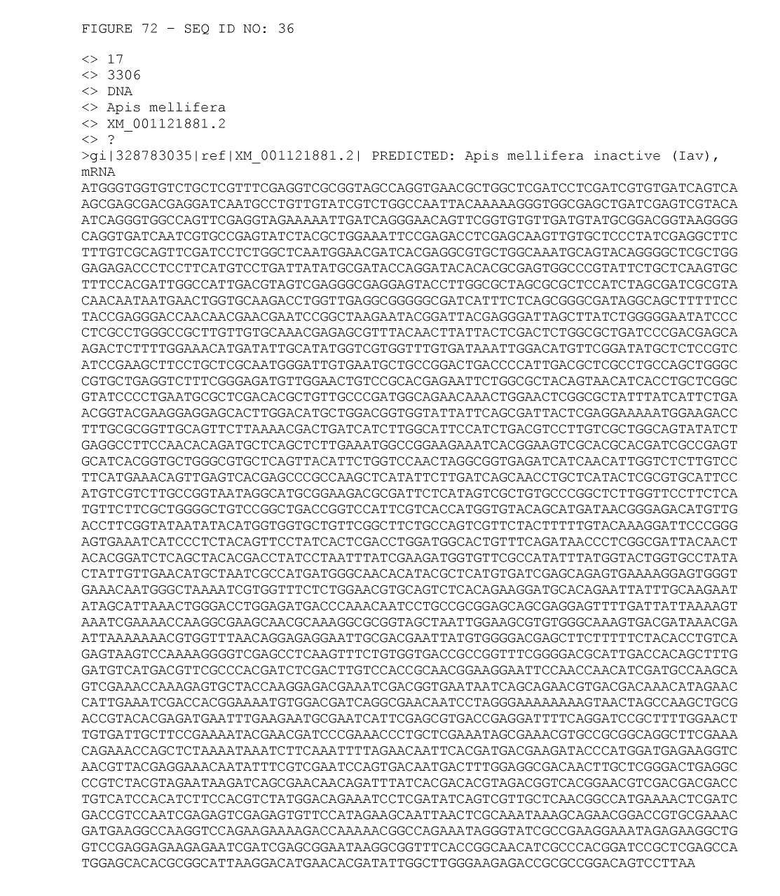

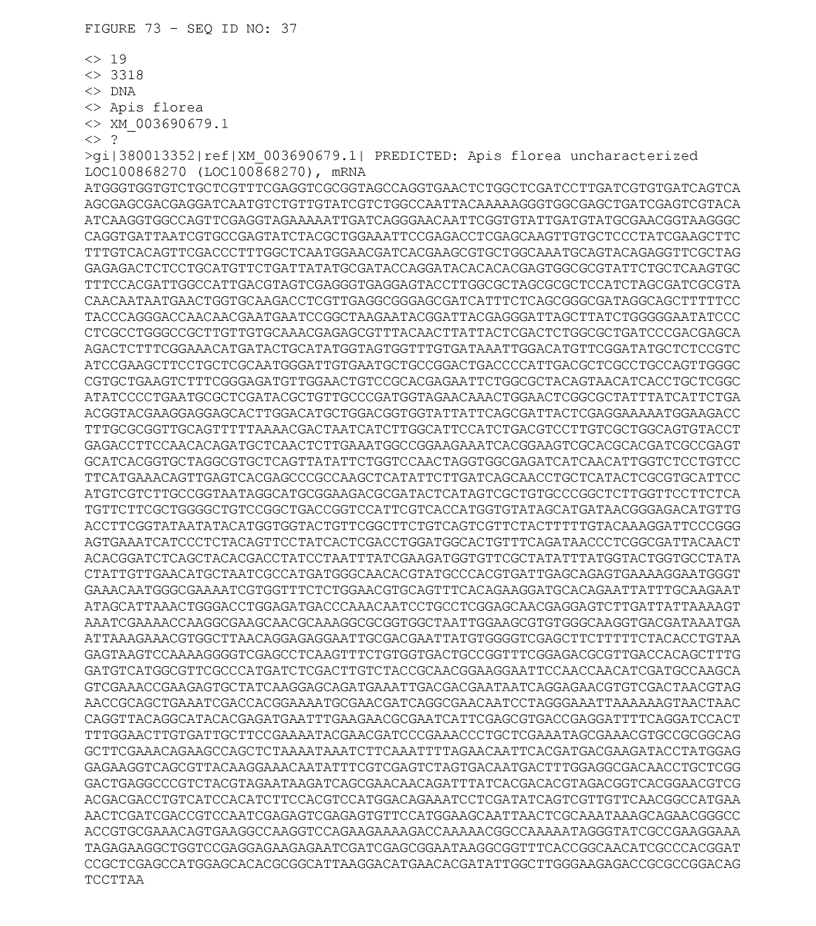

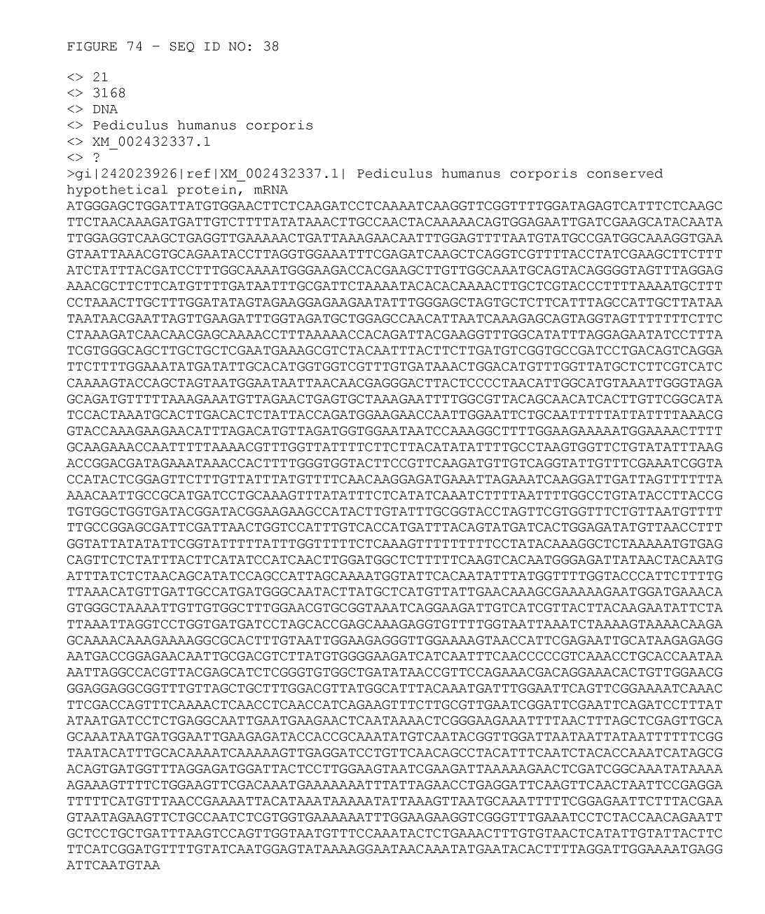

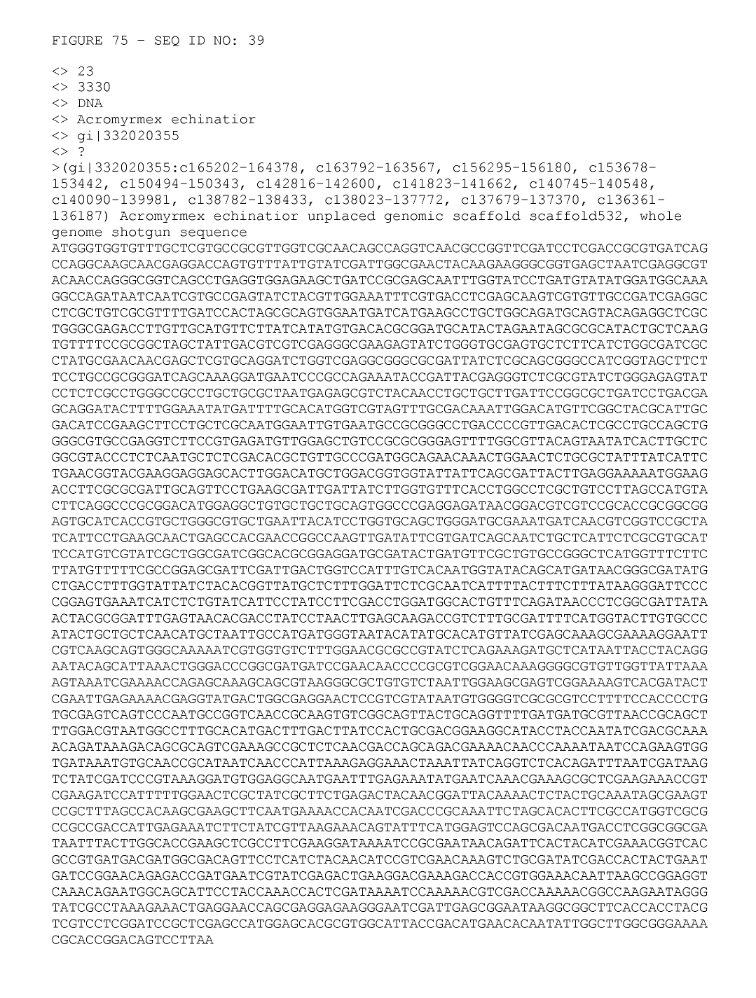

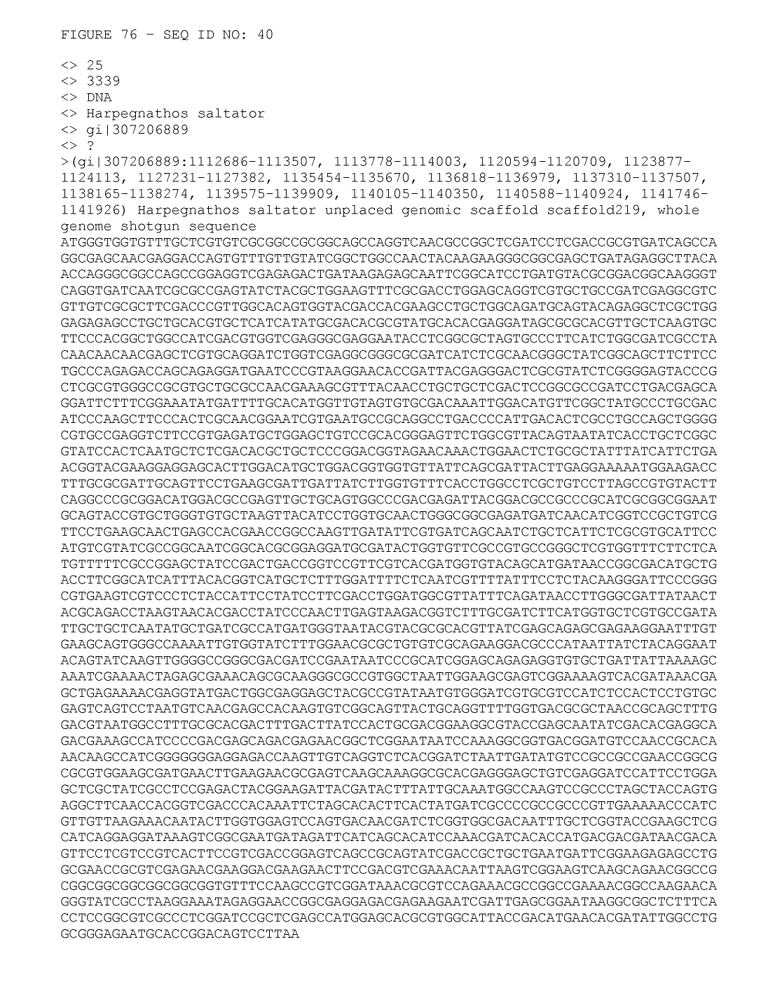

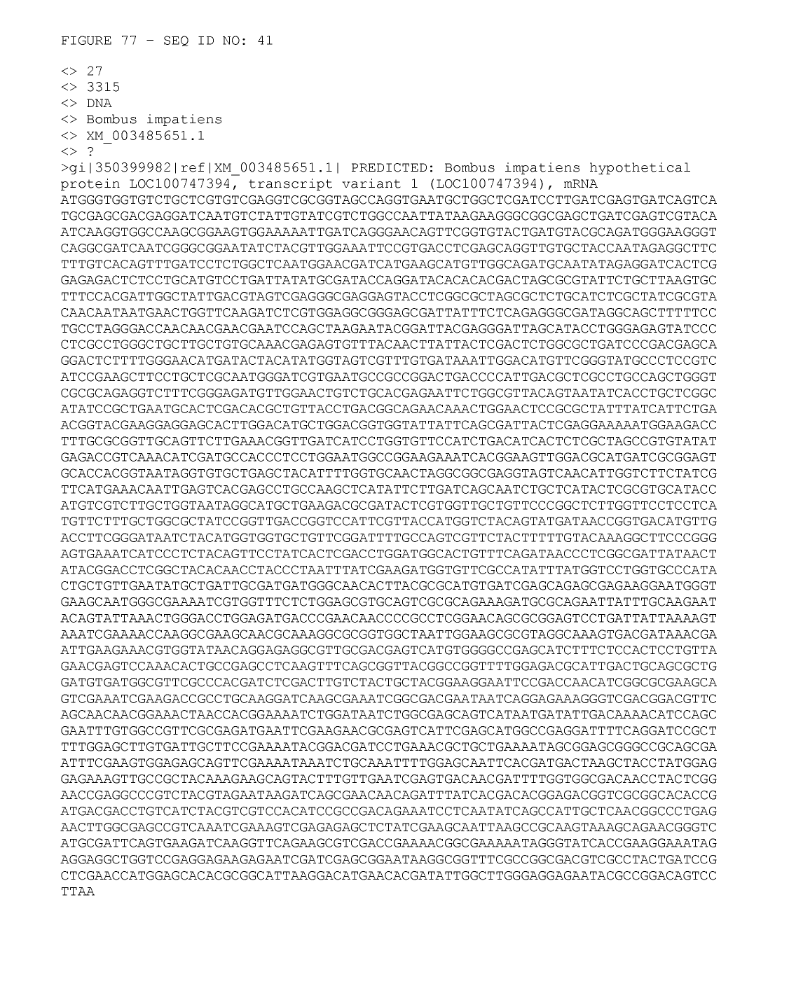

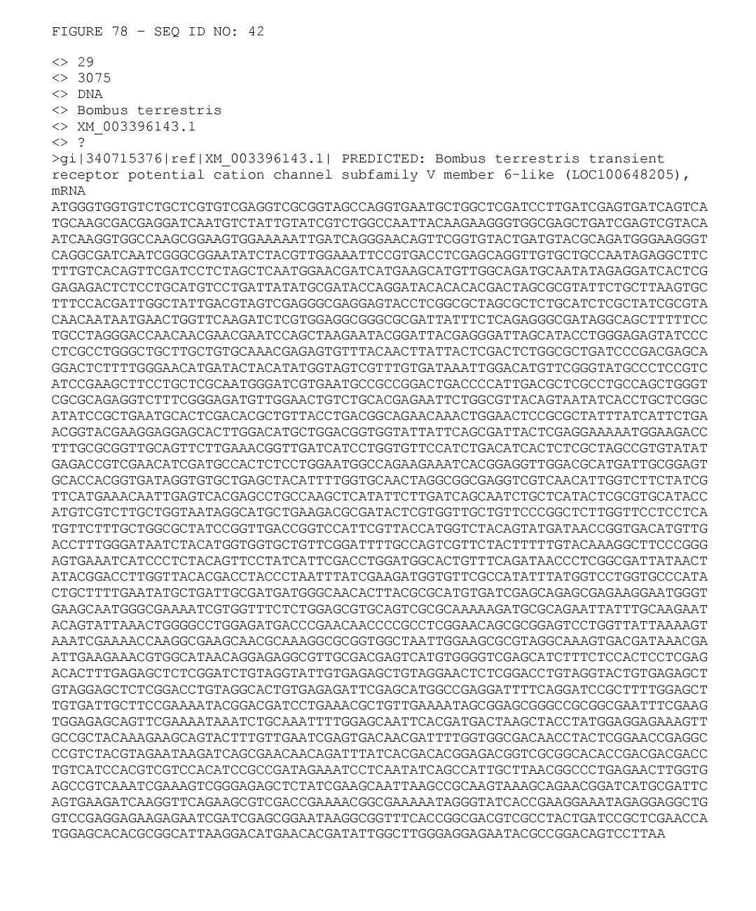

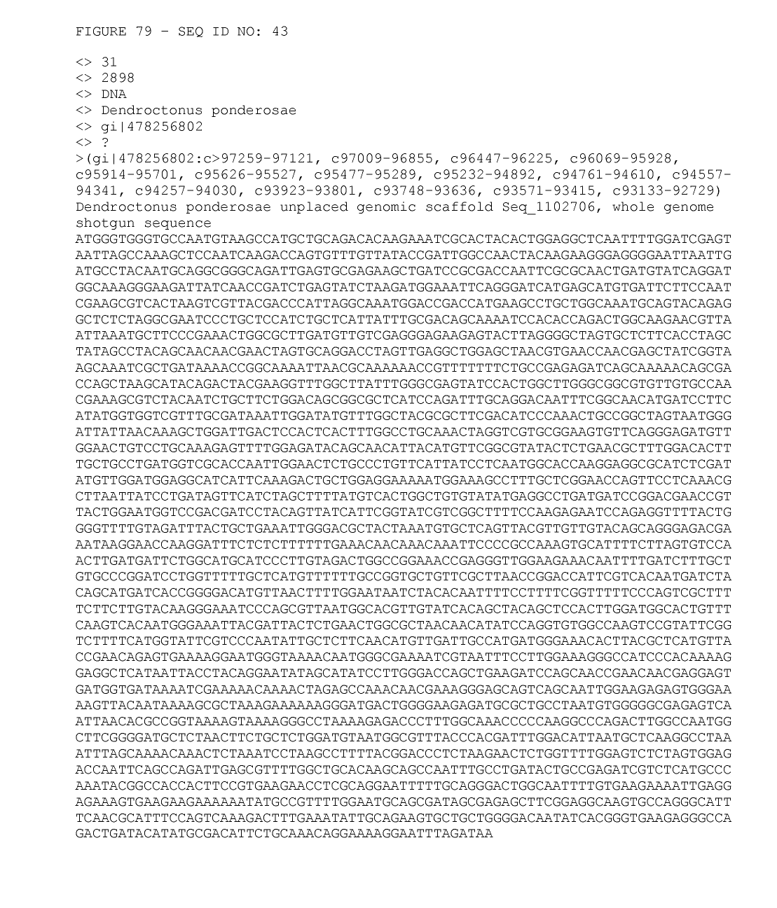

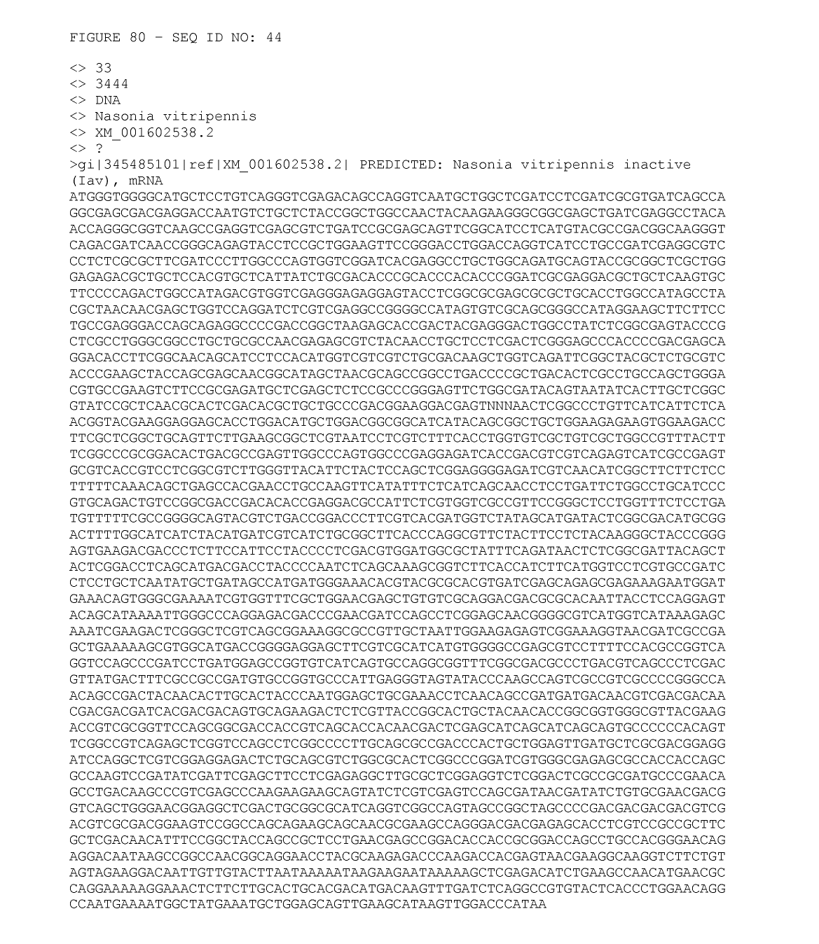

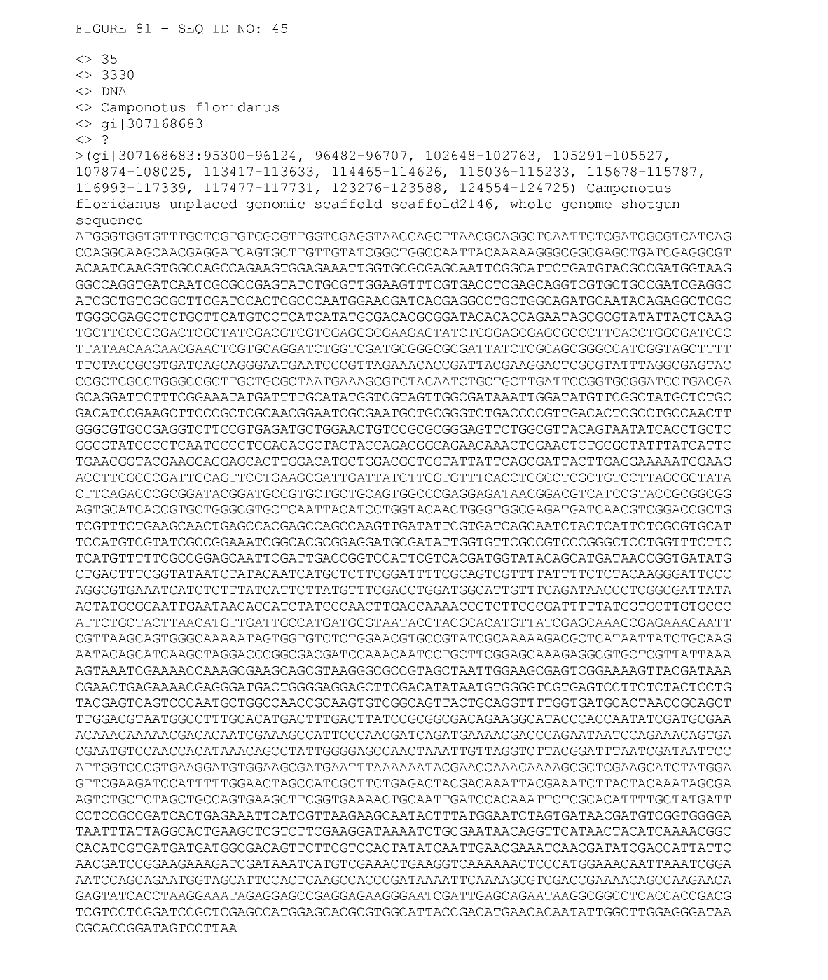

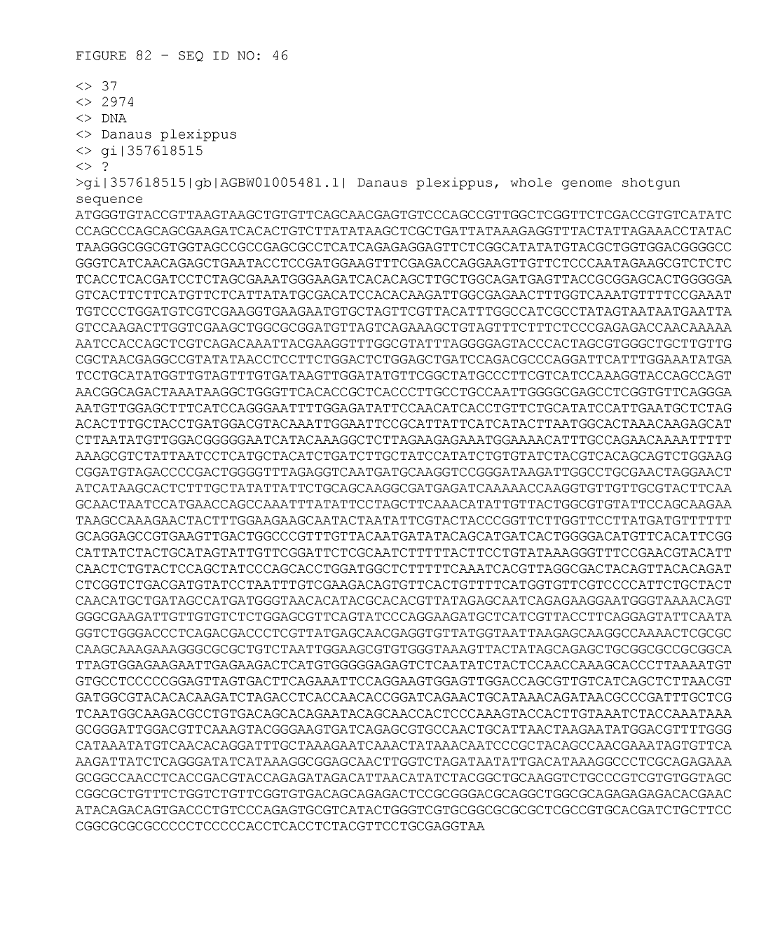

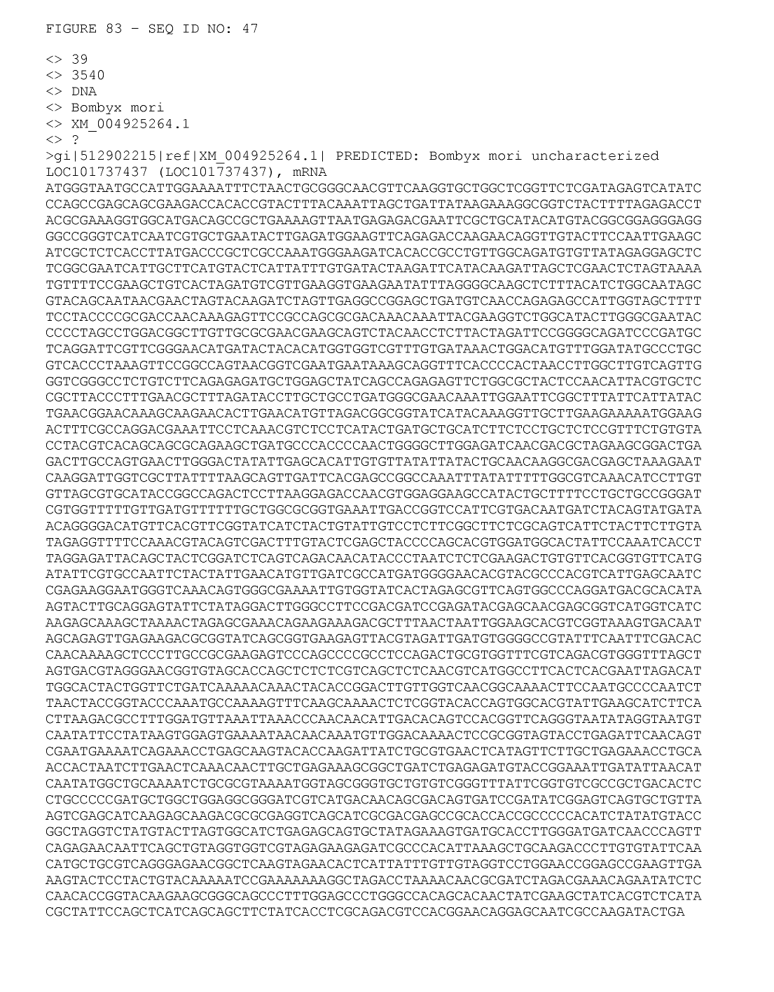

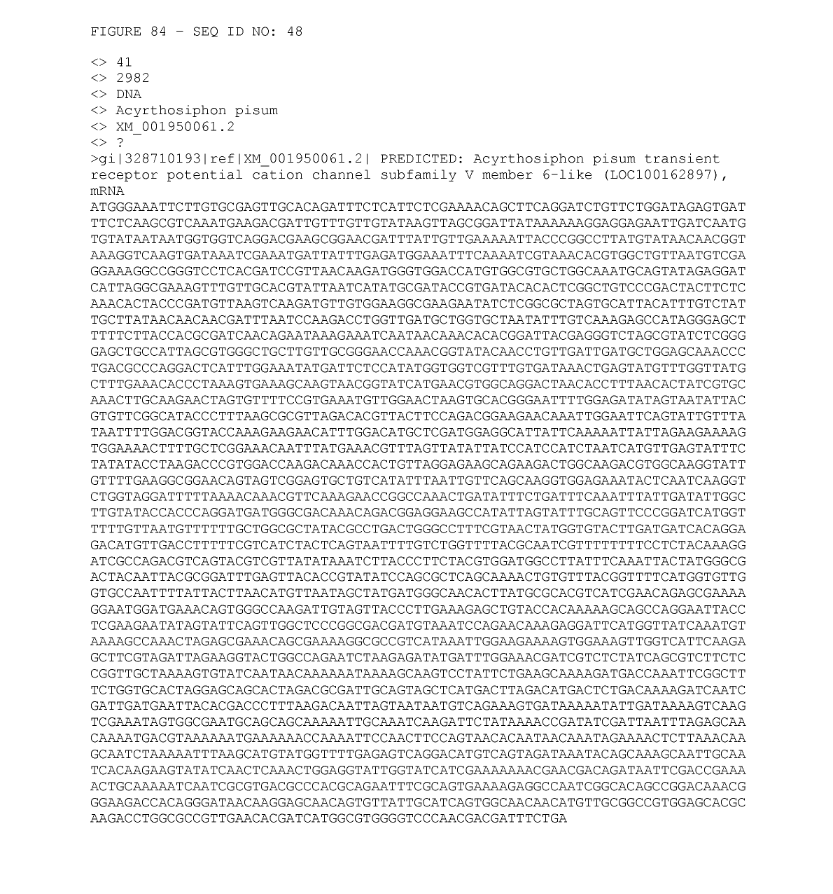

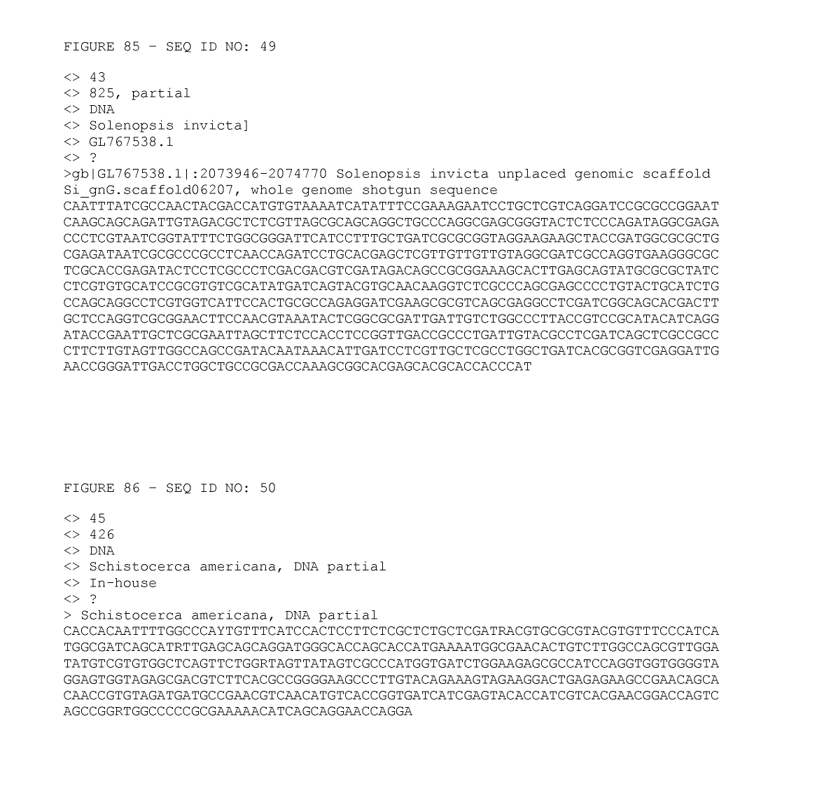

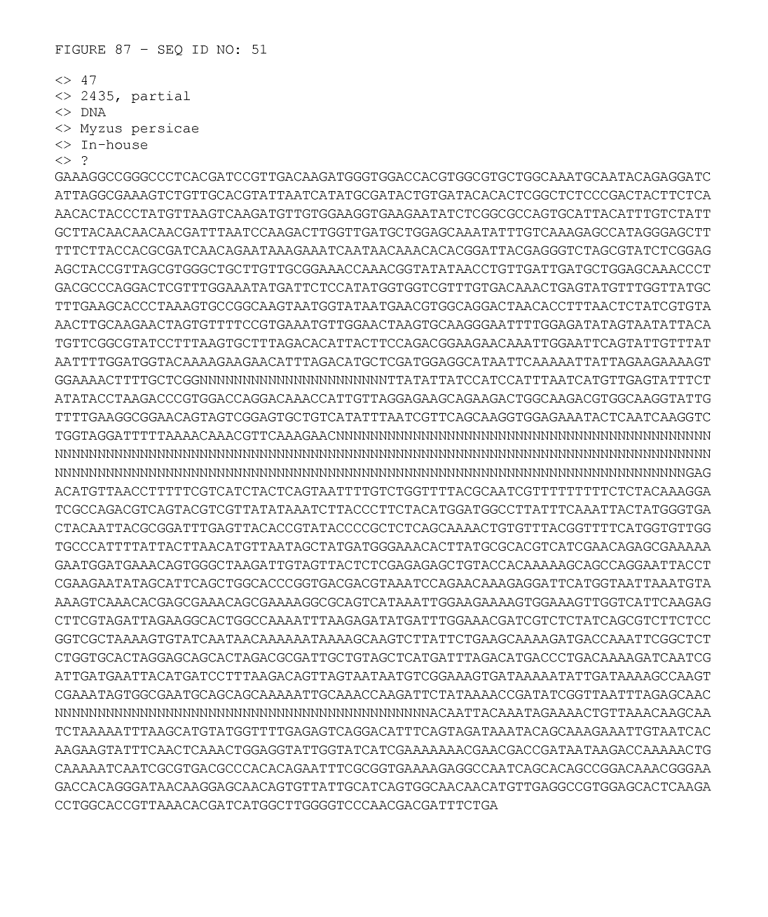

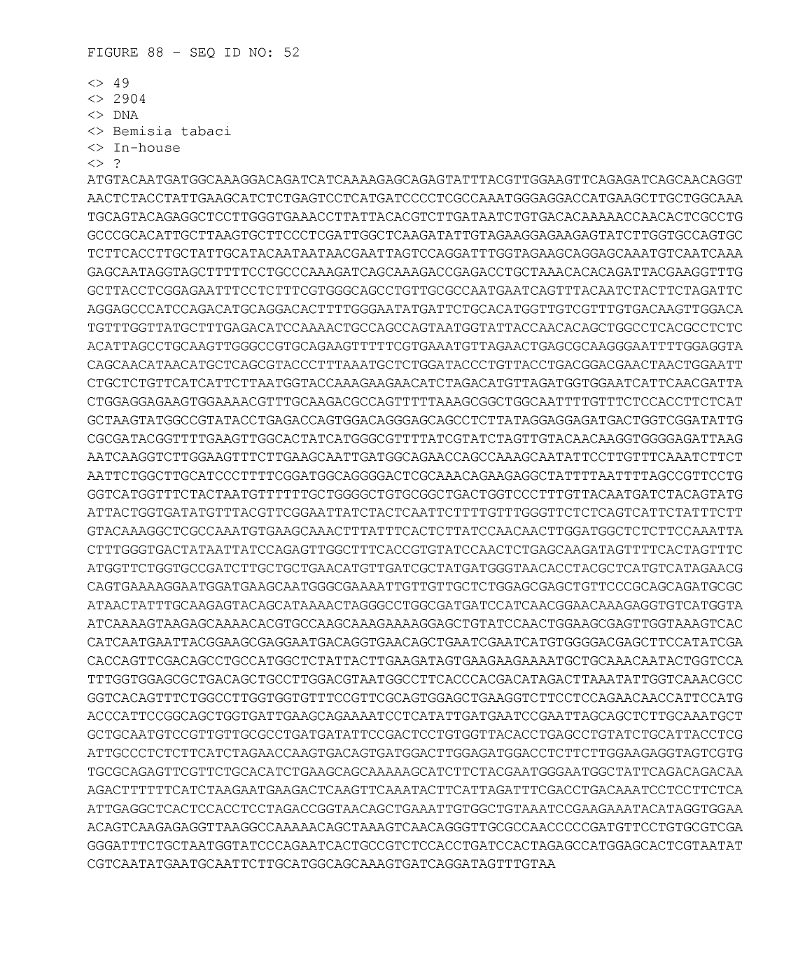

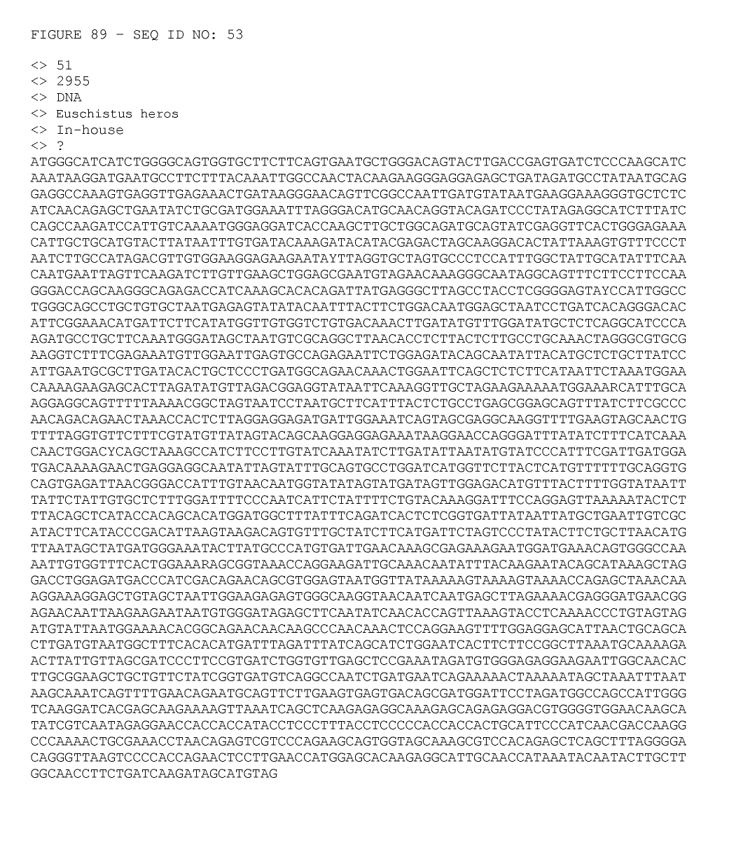

In some embodiments, the Inactive nucleic acid sequences may be selected from the group consisting of a) a polynucleotide molecule comprising a nucleic acid molecule having a sequence selected from the group consisting of SEQ ID NOS: 28-53 (FIGS. 64-89); b) a polynucleotide molecule having at least about 70% sequence identity to the sequence of SEQ ID NOS: 28-53 (FIGS. 64-89); and c) a fragment of the polynucleotide molecule of a) or b). Such fragments can be a UTR, a core promoter, an intron, an enhancer, a cis-element, or any other regulatory element.

Human, mouse, rat, or hamster TRPV1, TRPV2, TRPV3, TRPV4, TRPV5 or TRPV6

The disclosed polynucleotides are capable of providing for expression of Nanchung and Inactive proteins in the host cells.

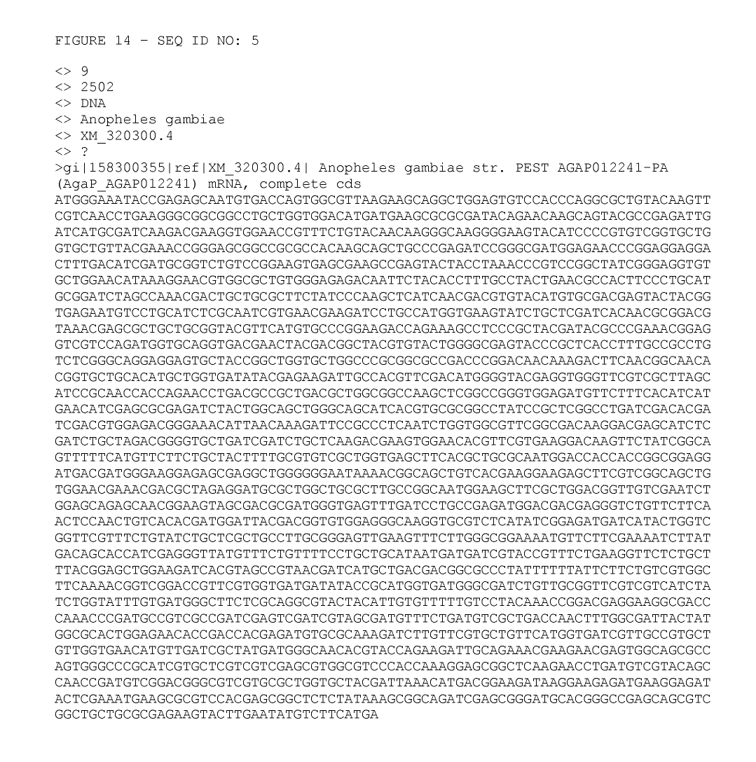

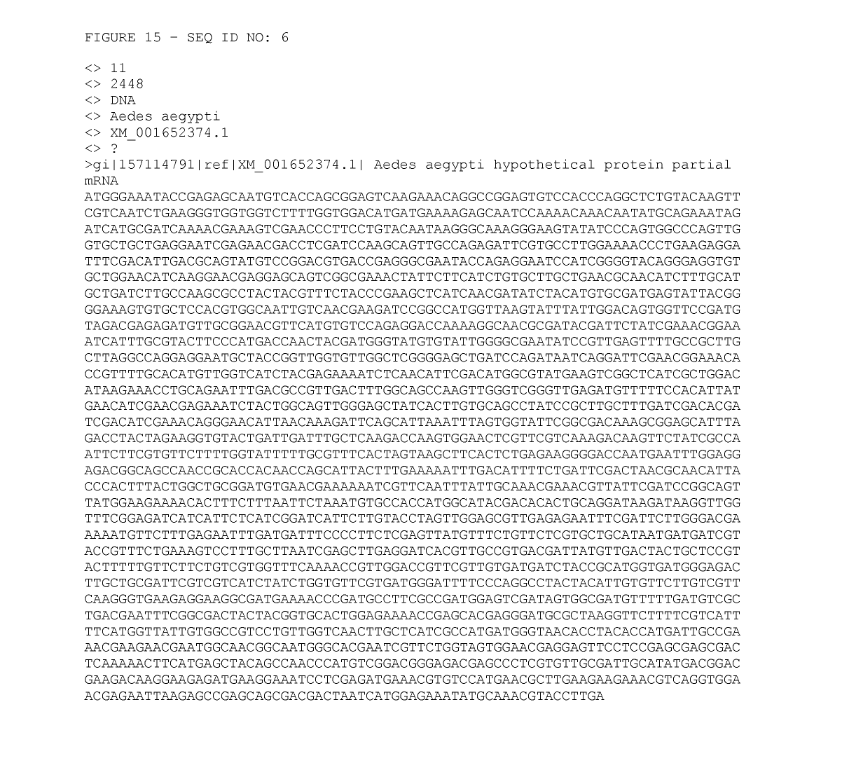

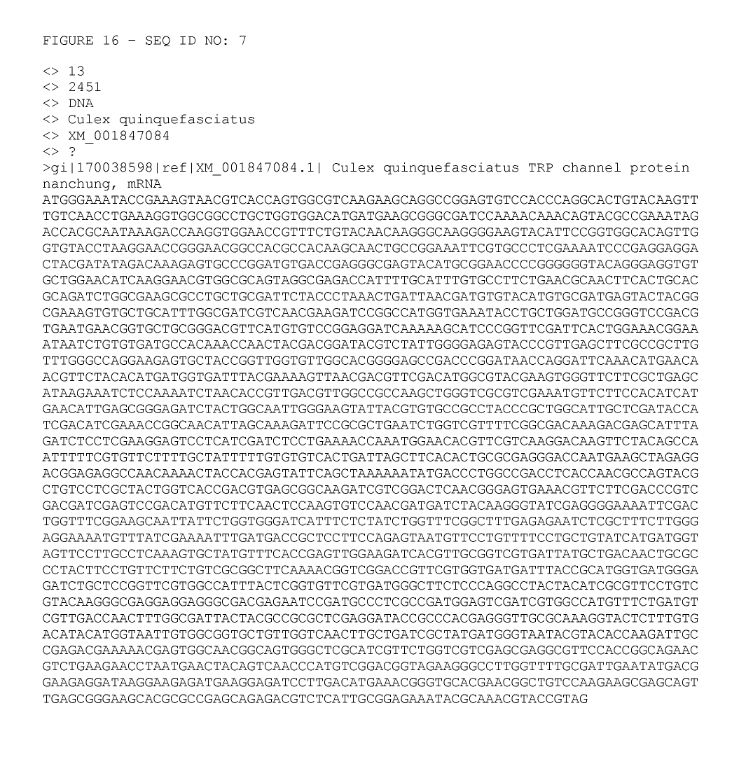

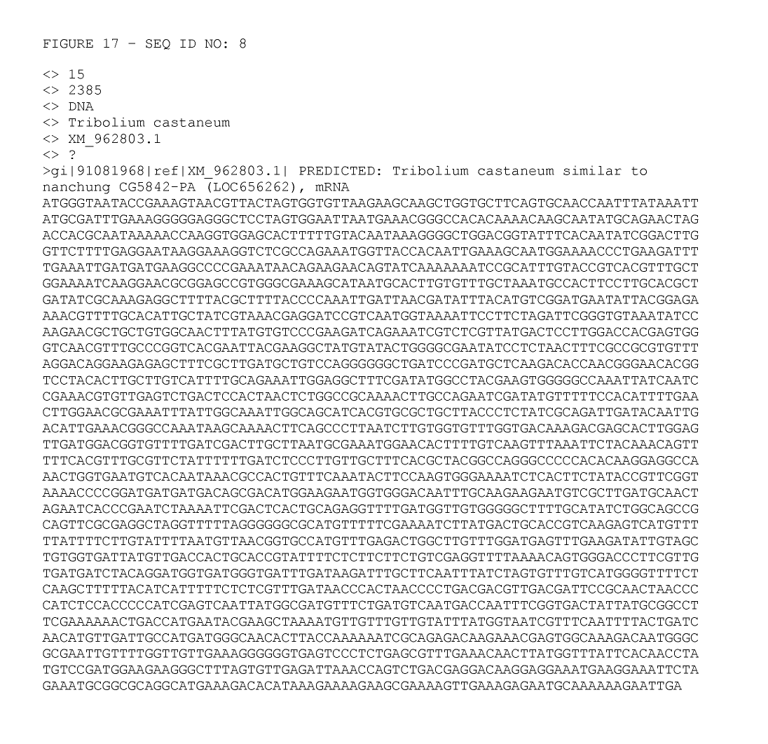

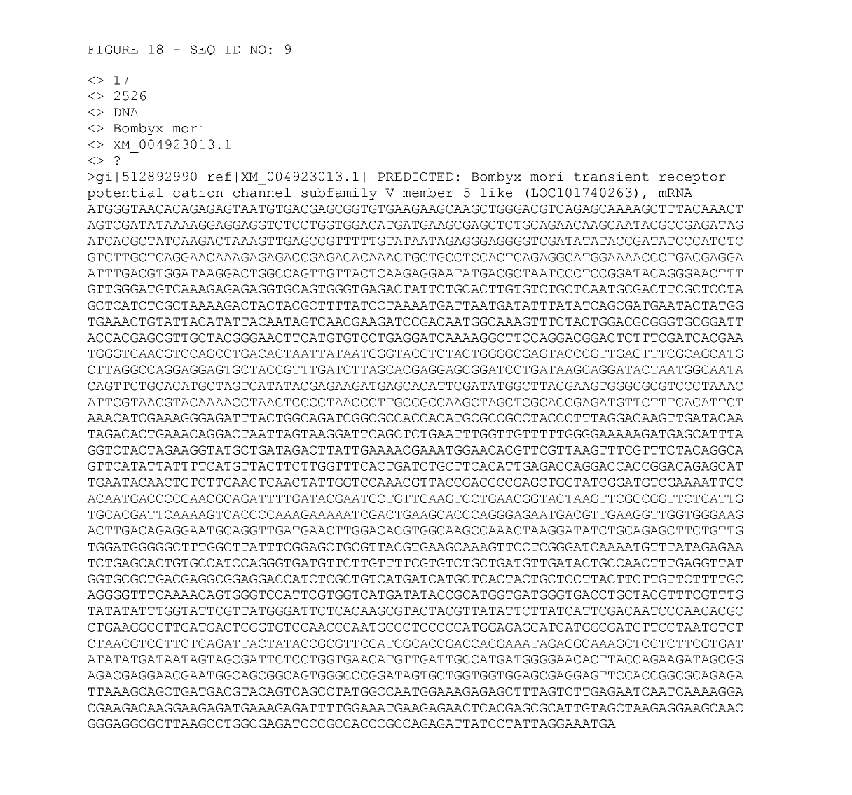

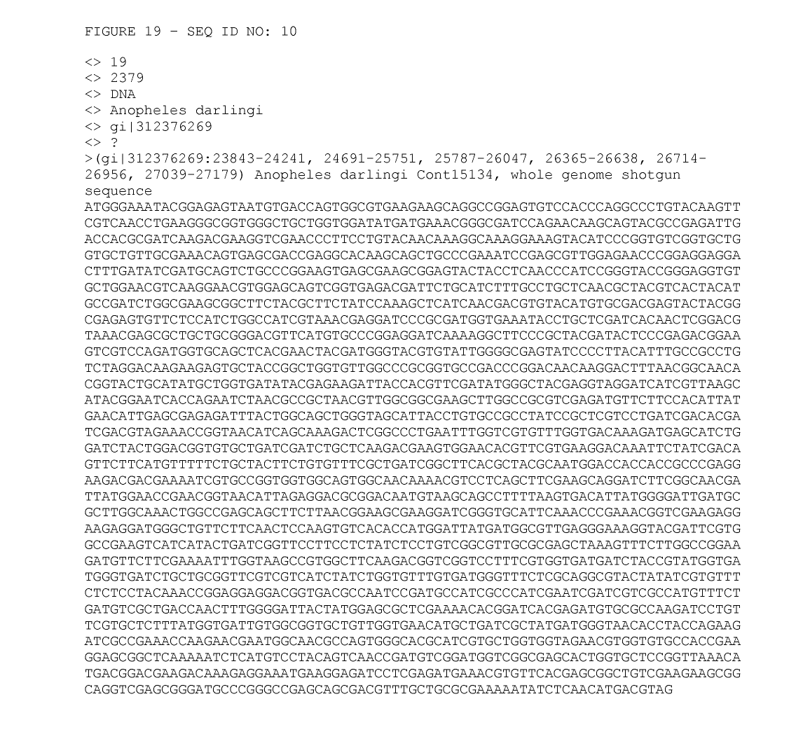

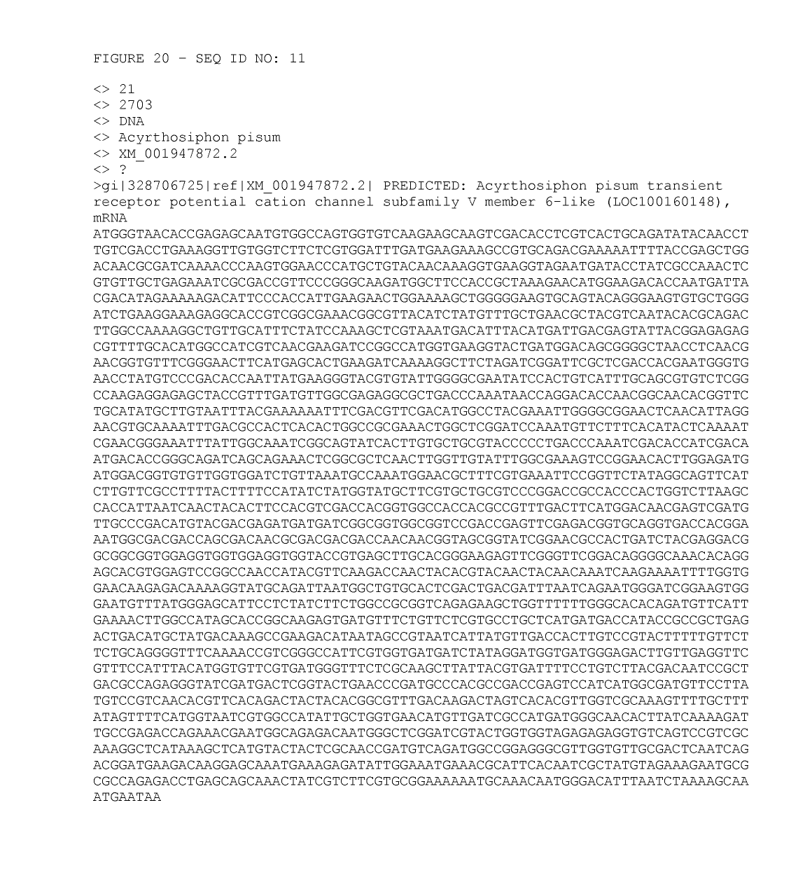

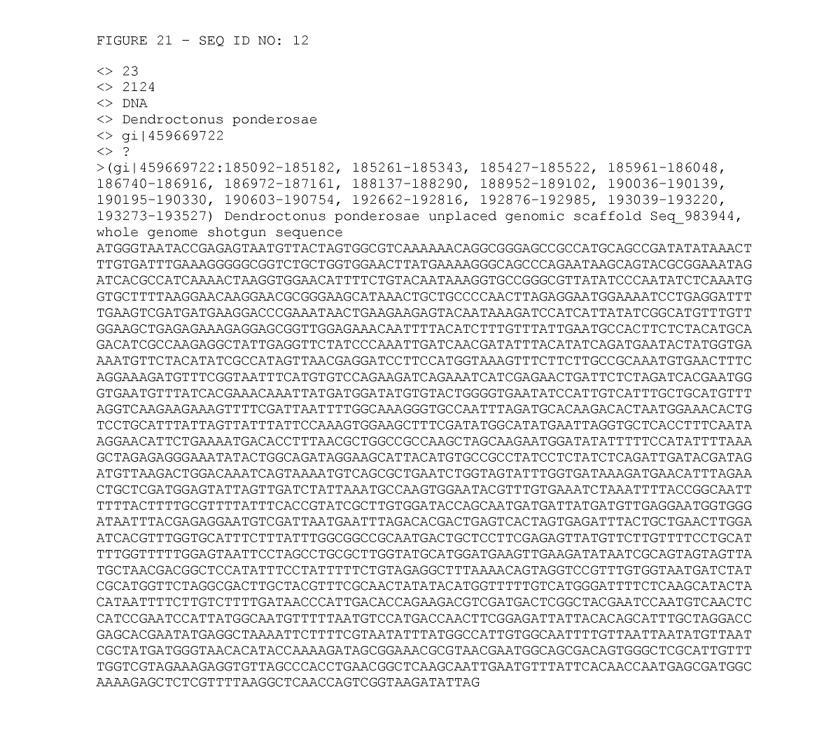

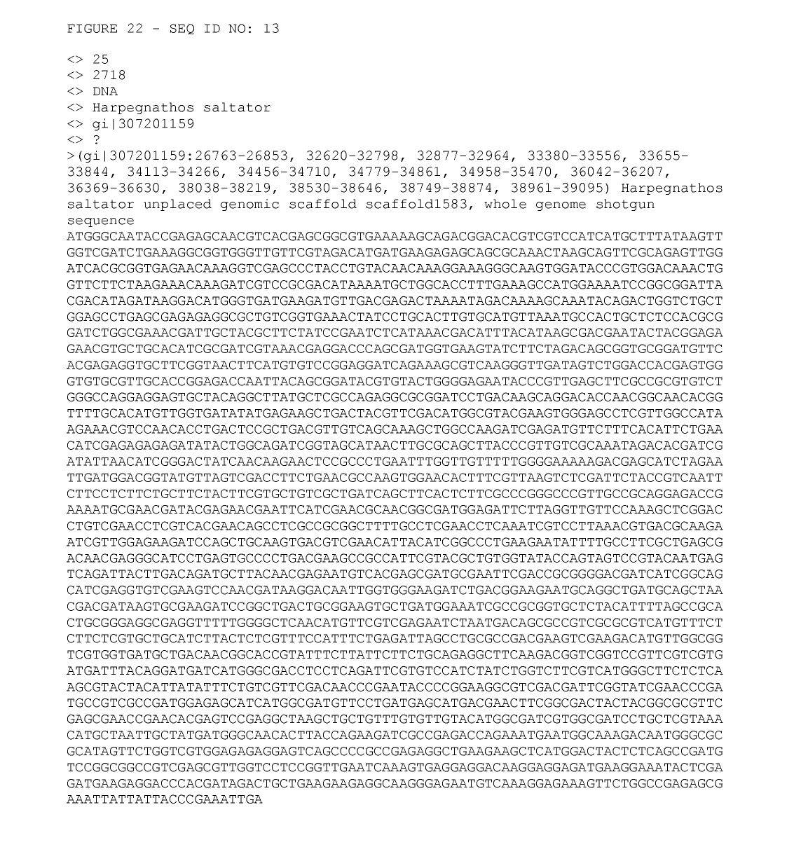

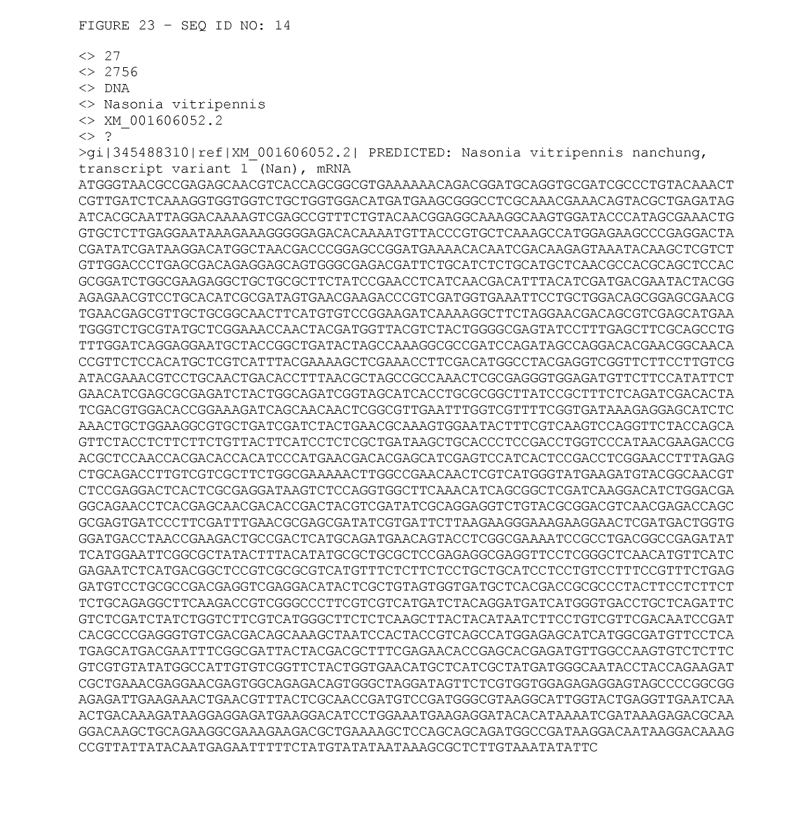

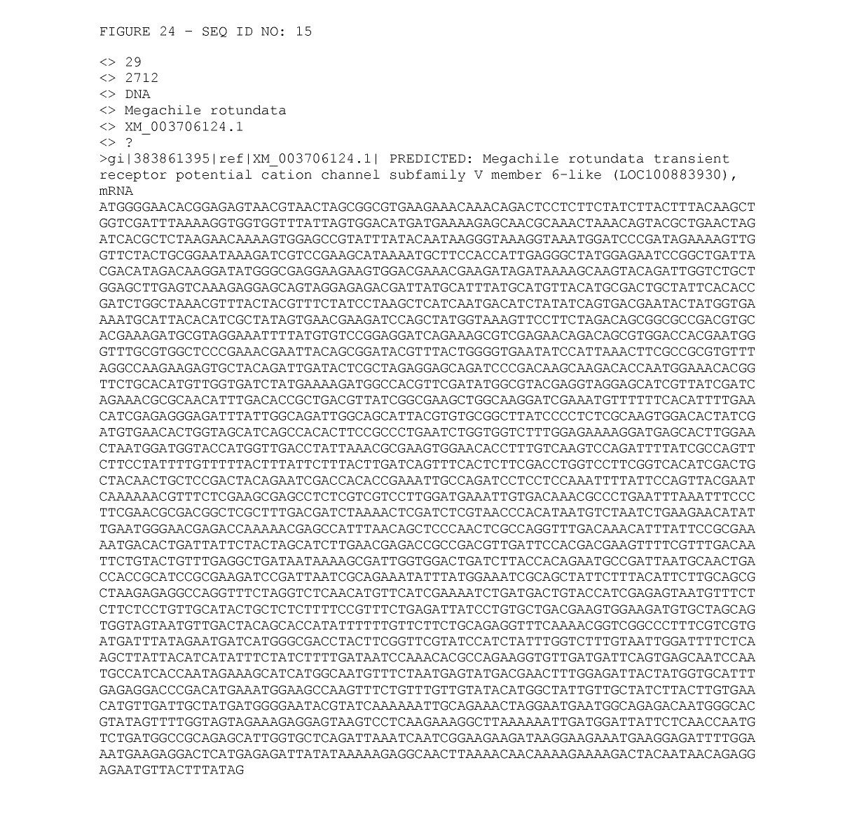

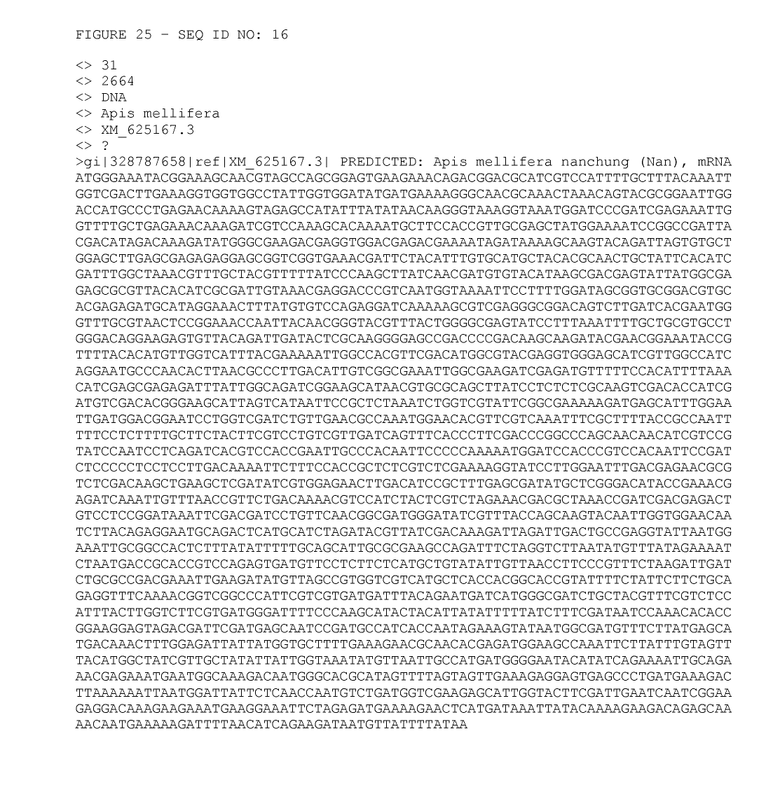

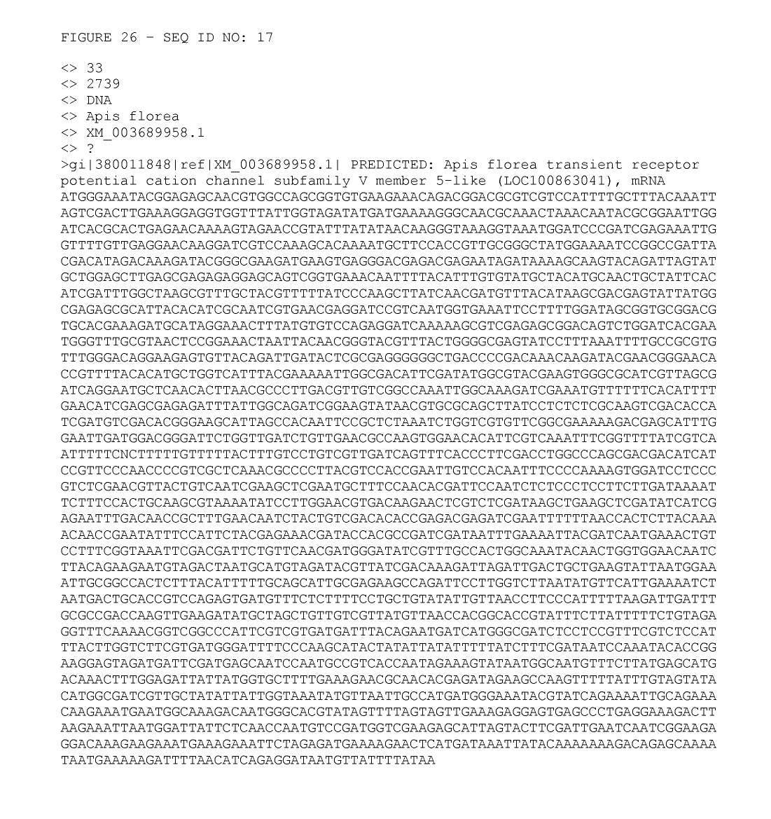

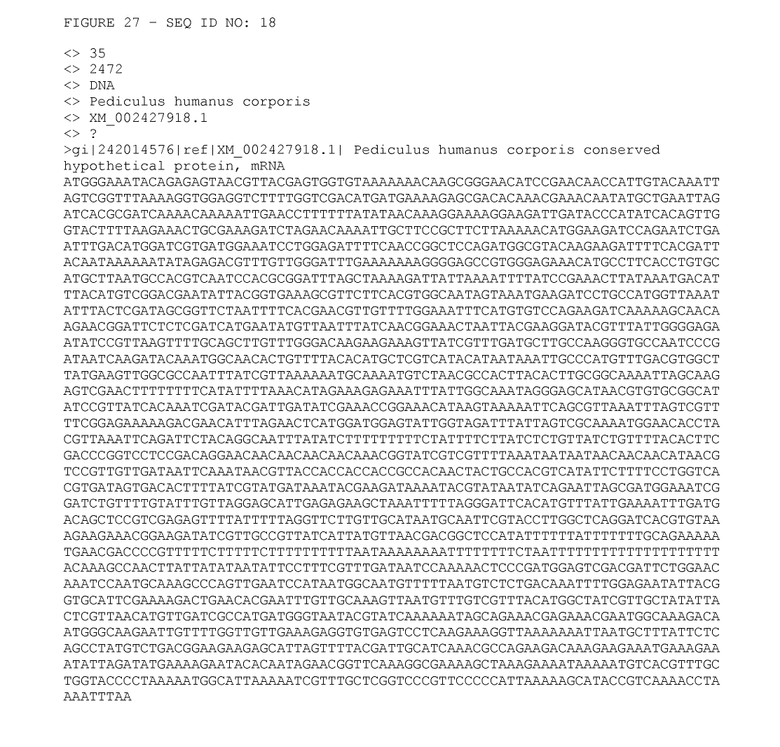

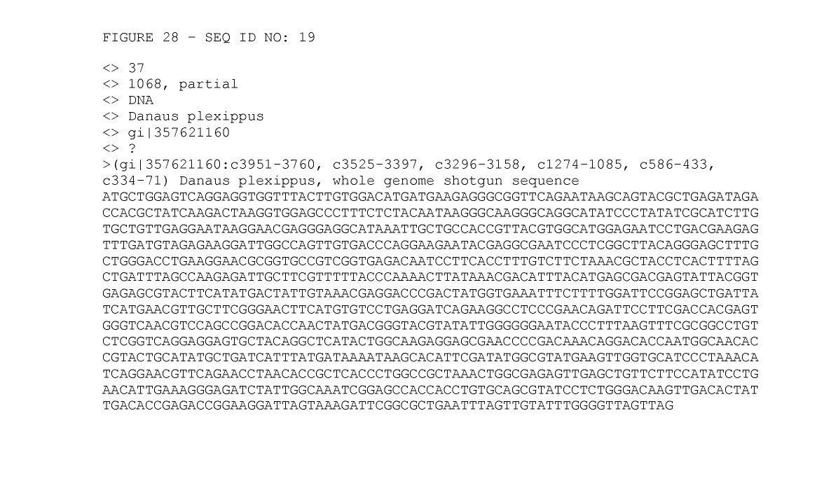

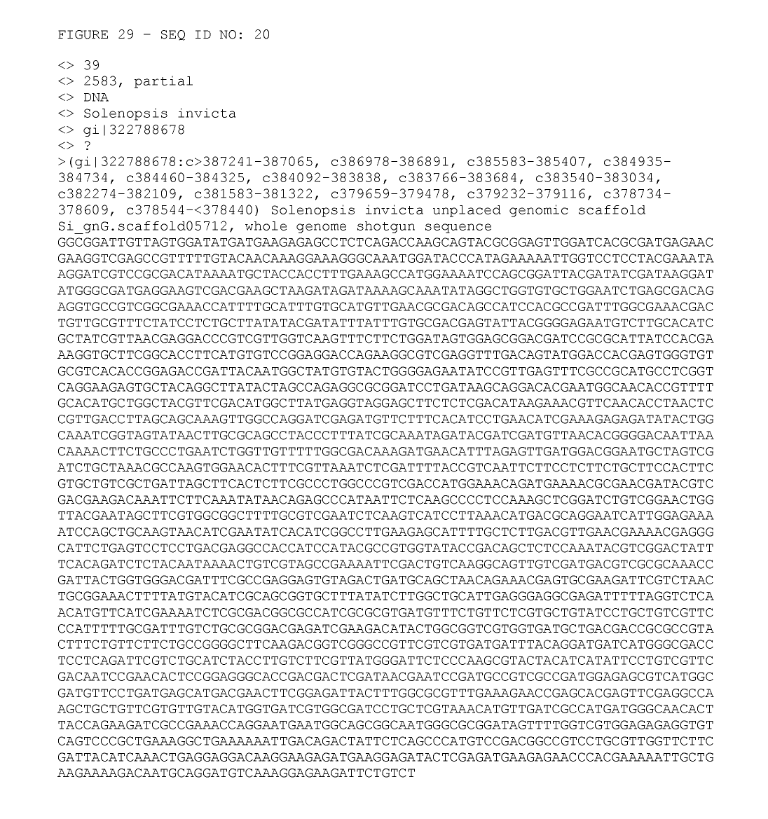

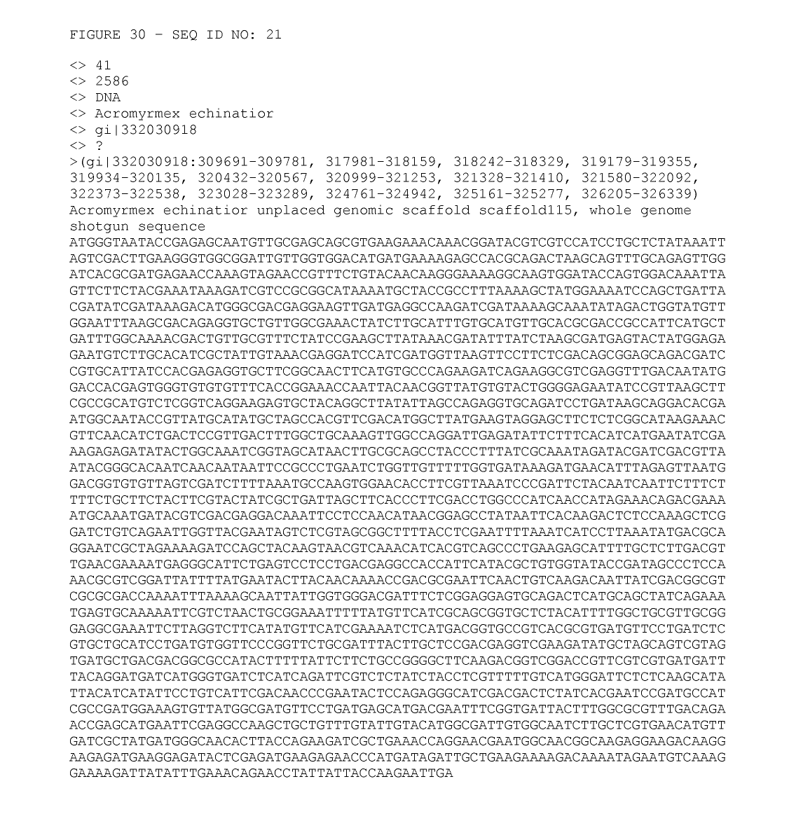

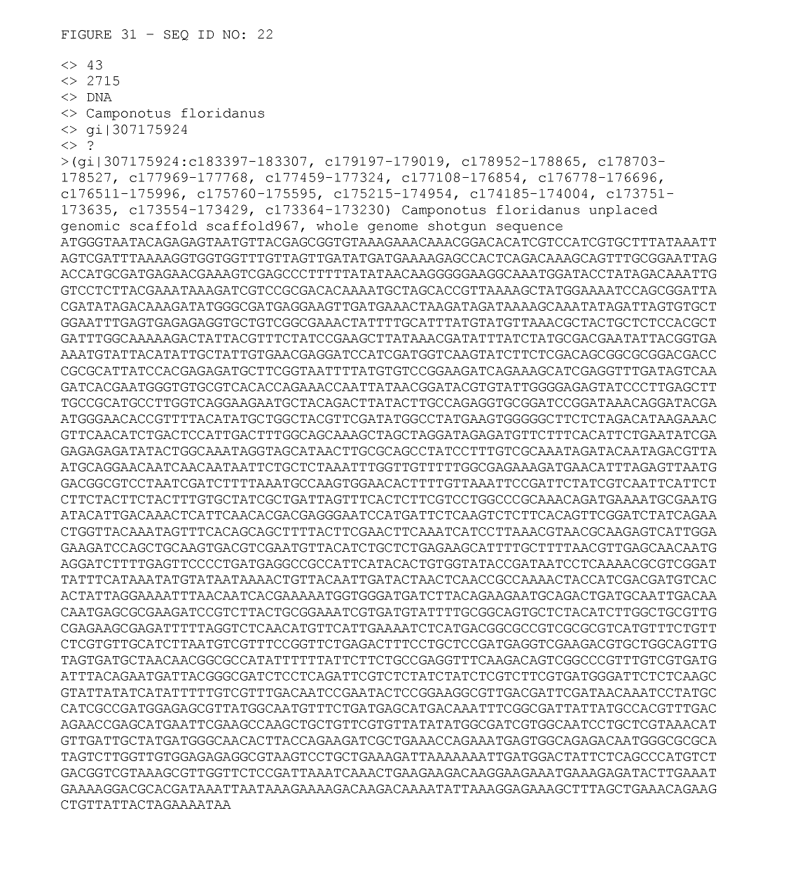

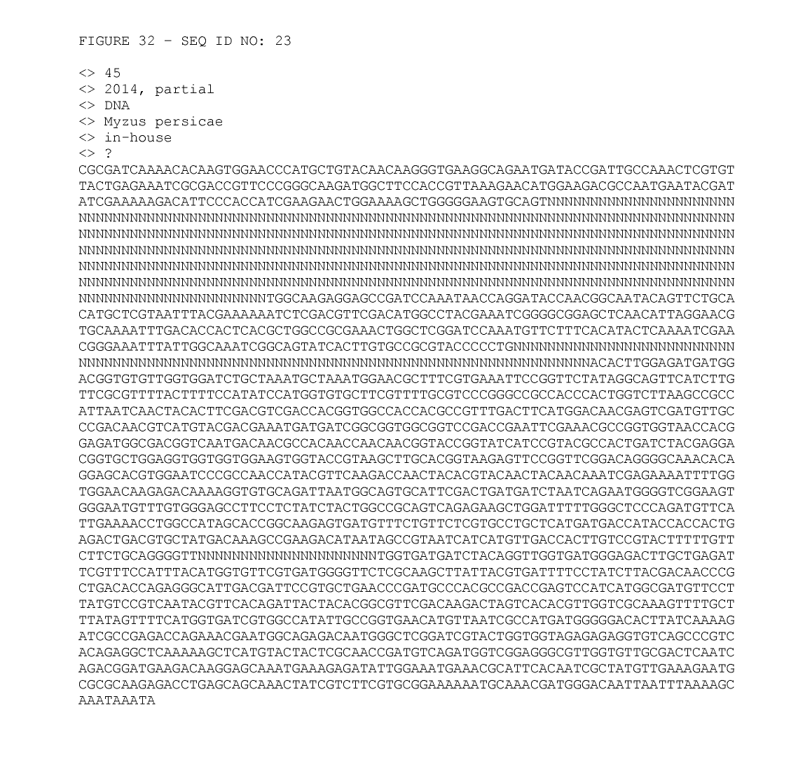

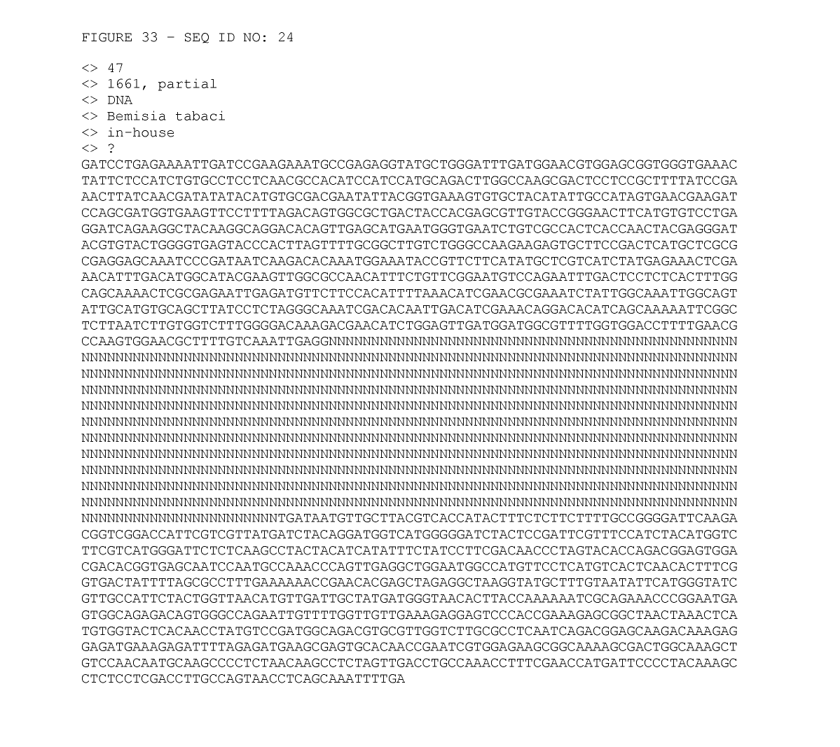

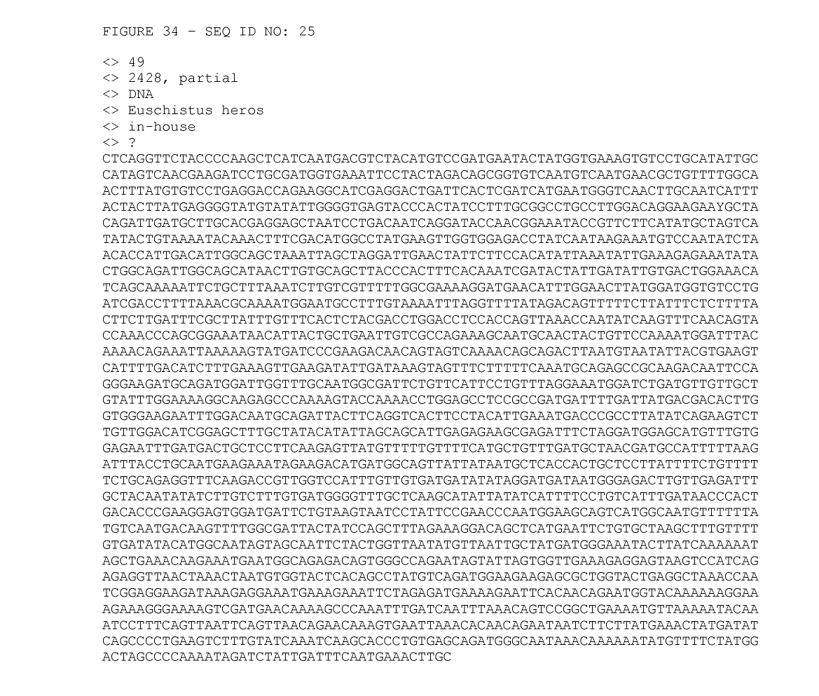

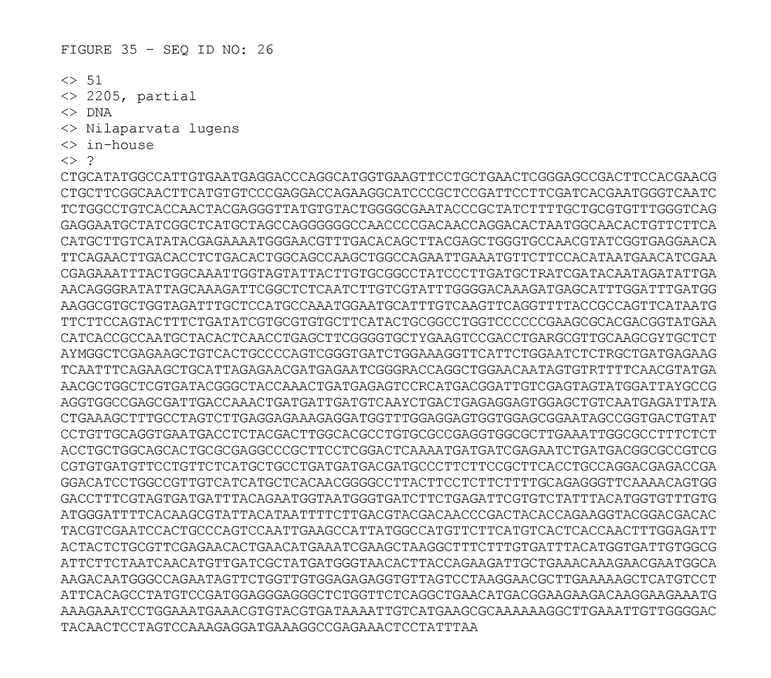

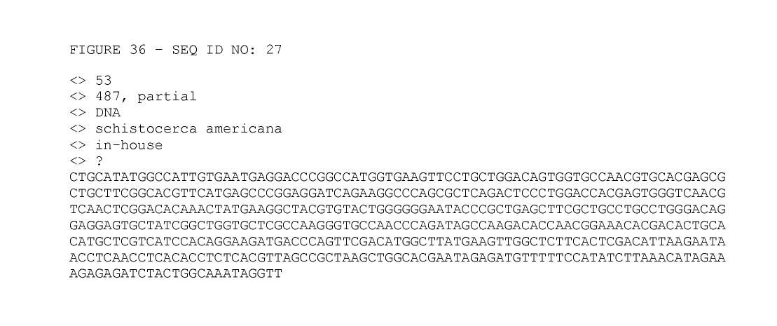

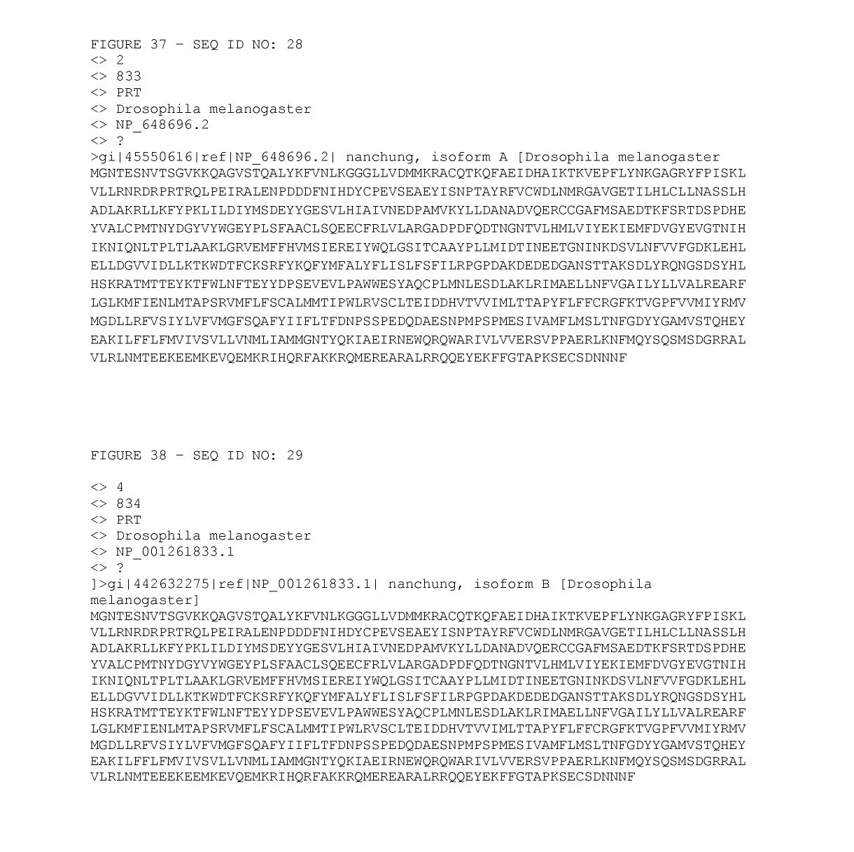

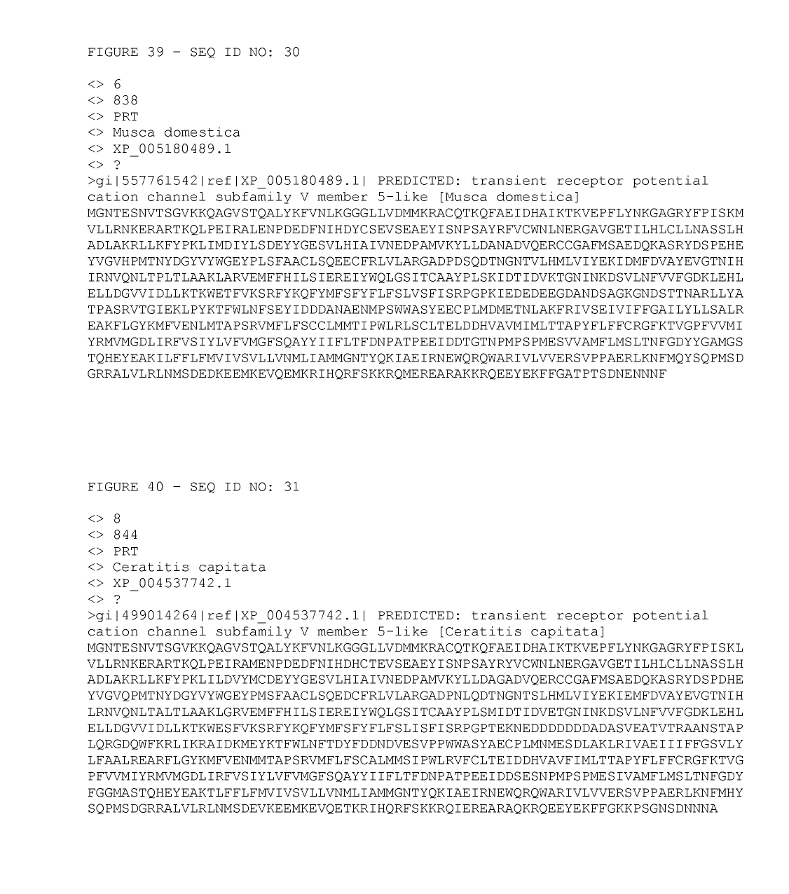

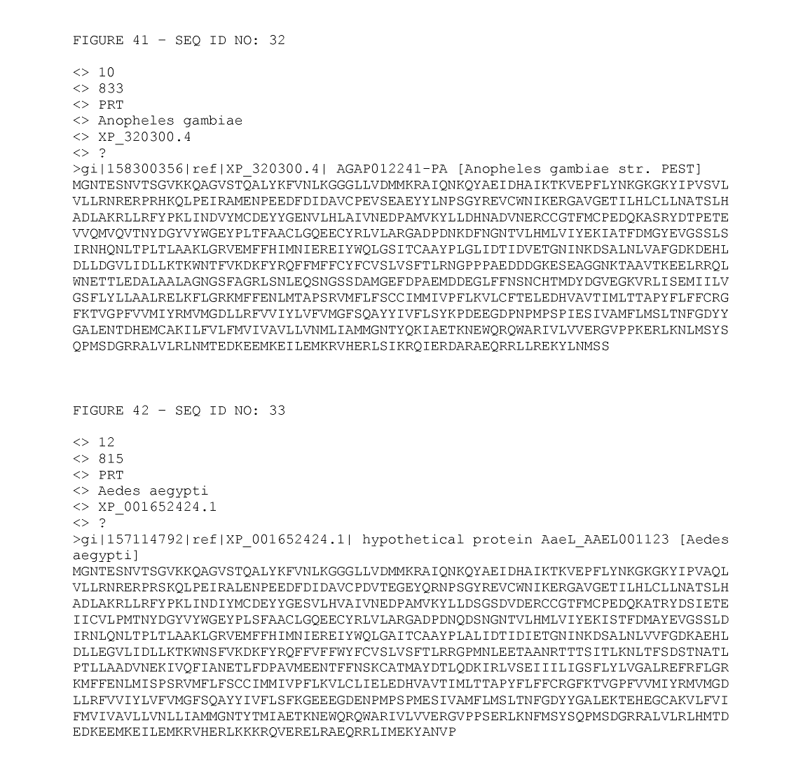

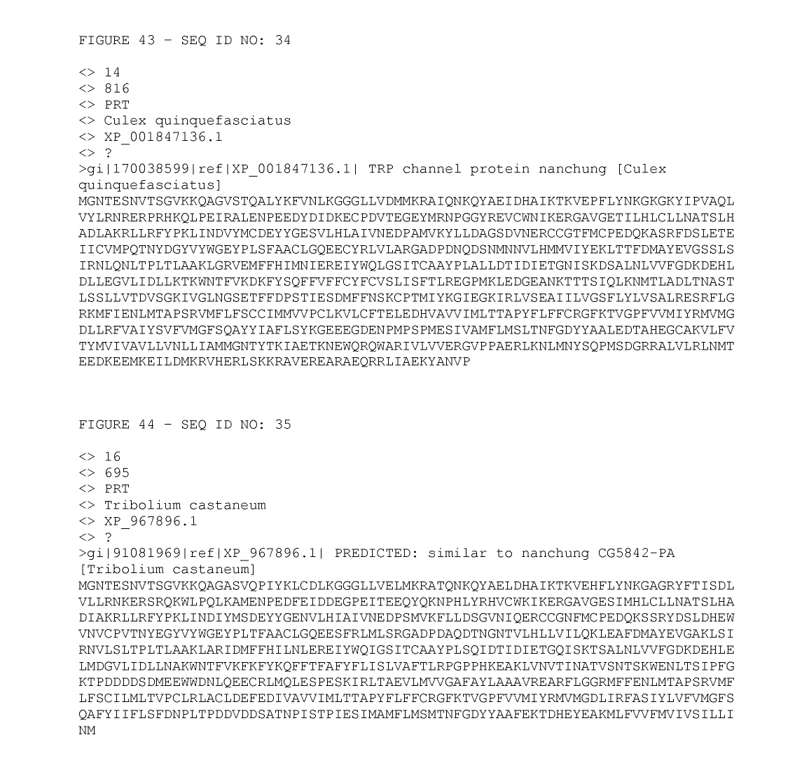

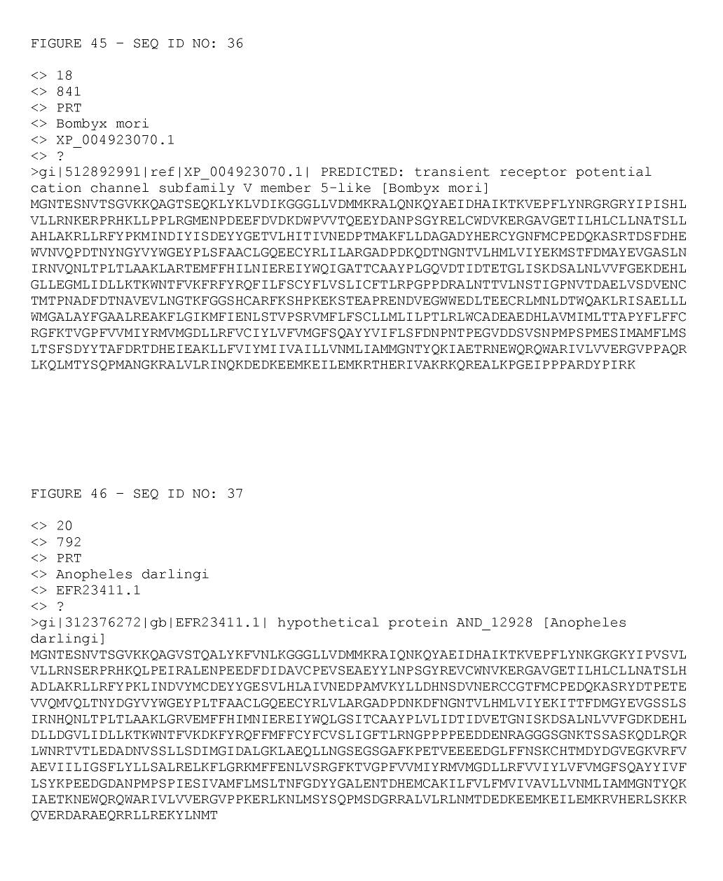

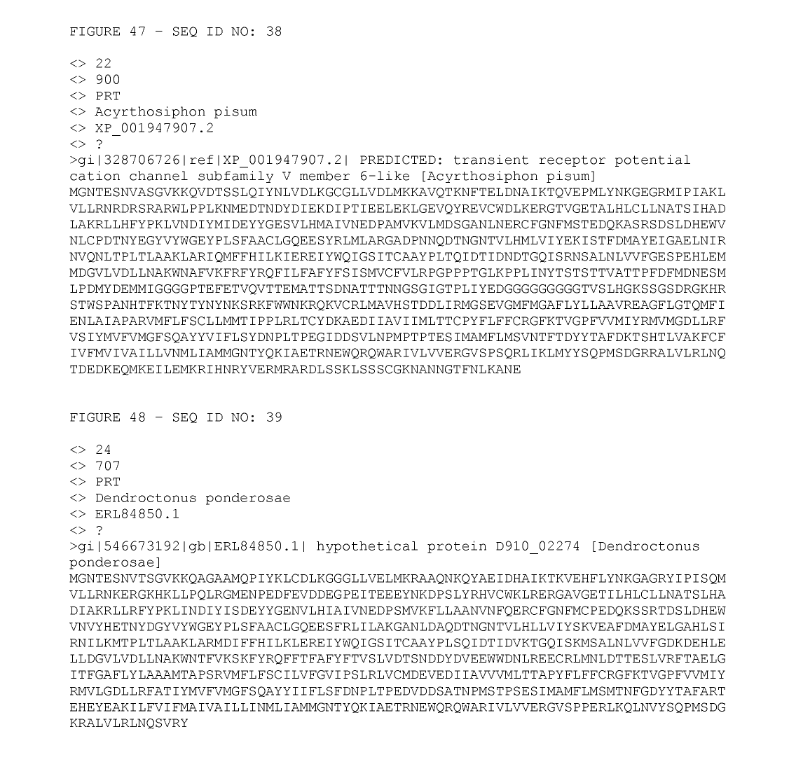

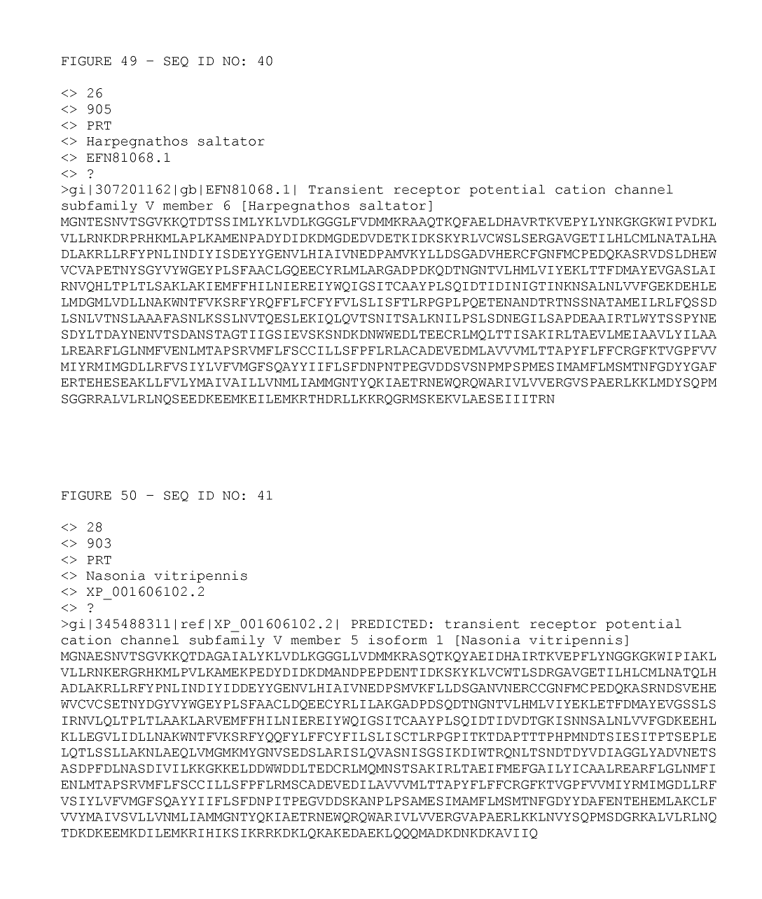

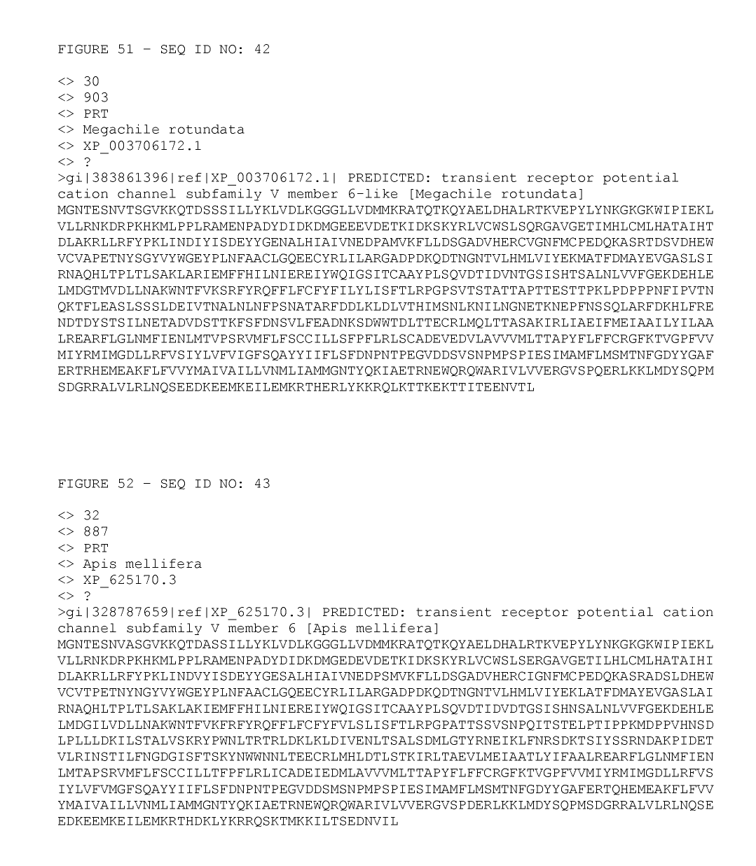

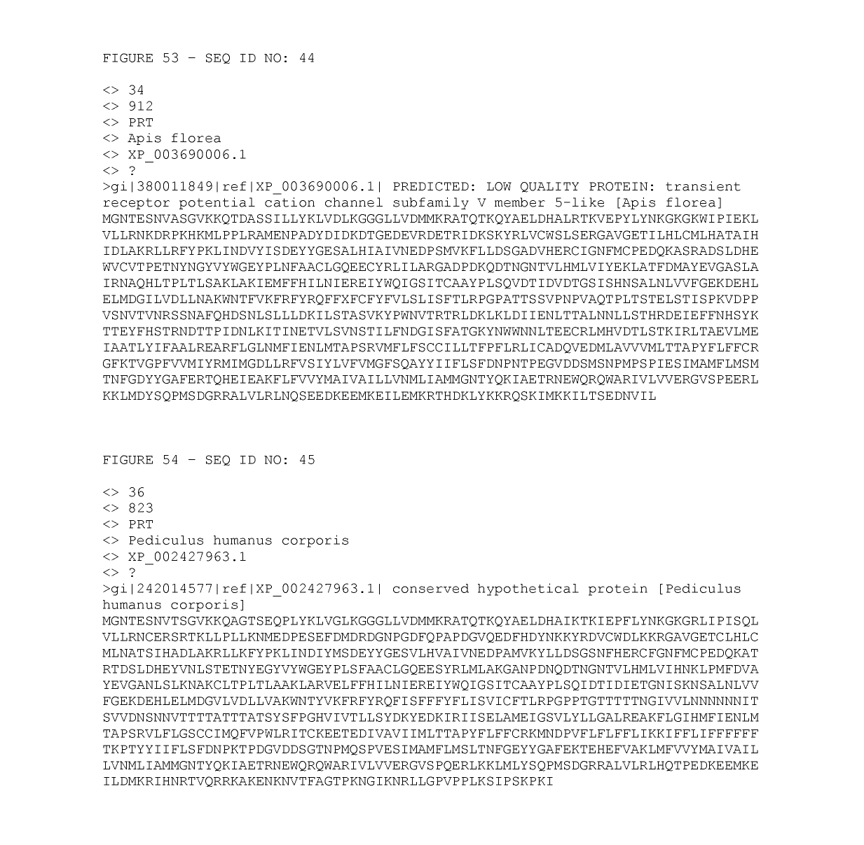

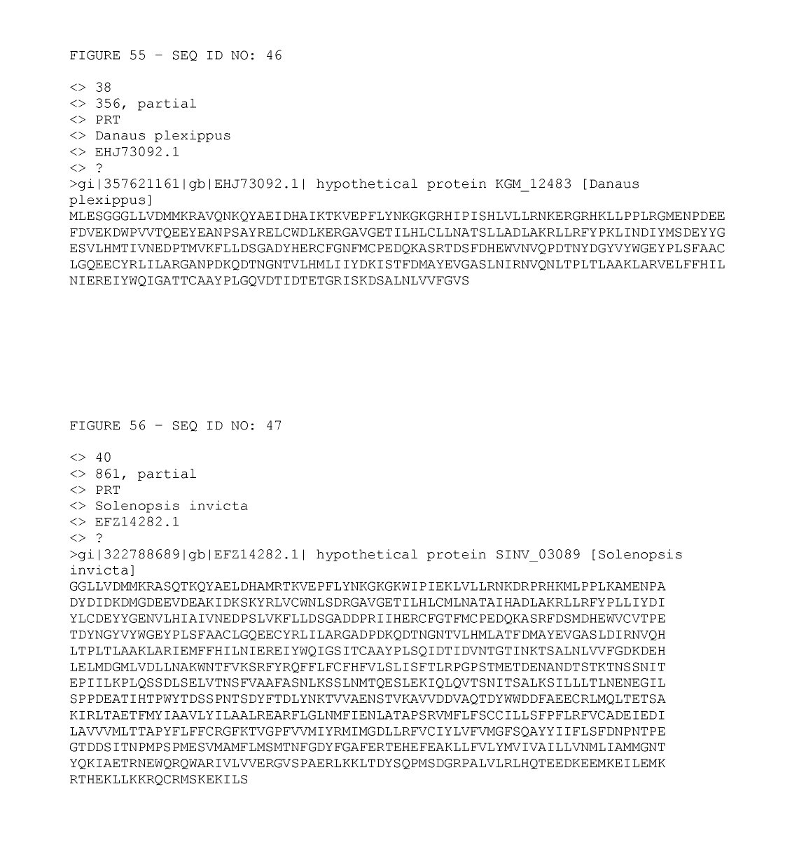

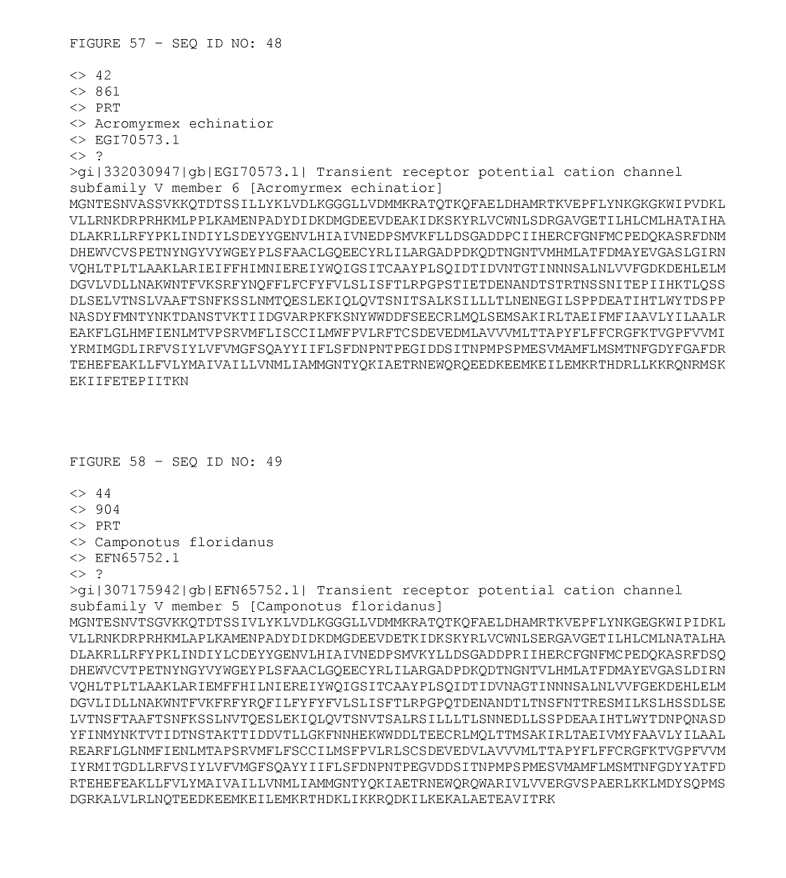

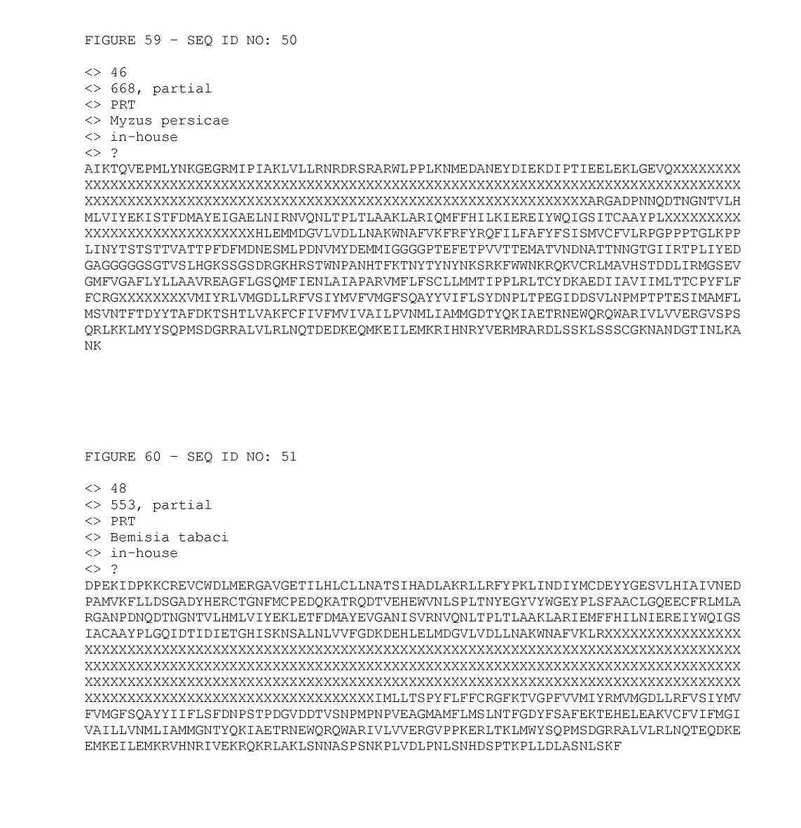

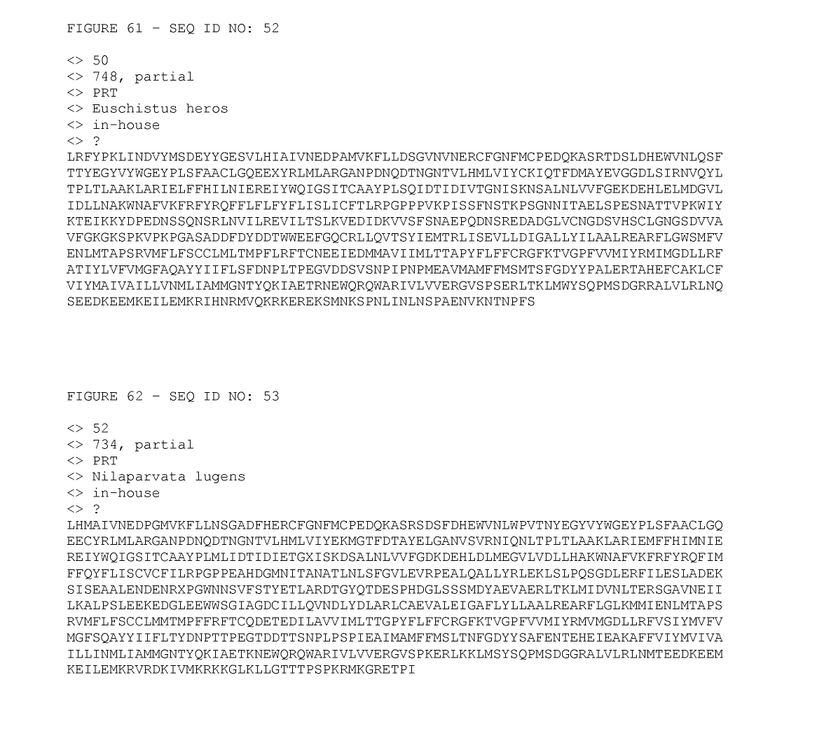

In certain embodiments, the Nanchung protein sequences may be selected from the group consisting of a) a polypeptide comprising an amino acid sequence having a sequence selected from the group consisting of SEQ ID NOS: 54-80 (FIGS. 37-63); b) a polypeptide having at least 70% sequence identity to the SEQ ID NOS: 54-80 (FIGS. 37-63); and c) a fragment or a conservative variant of the polypeptide of a) or b).

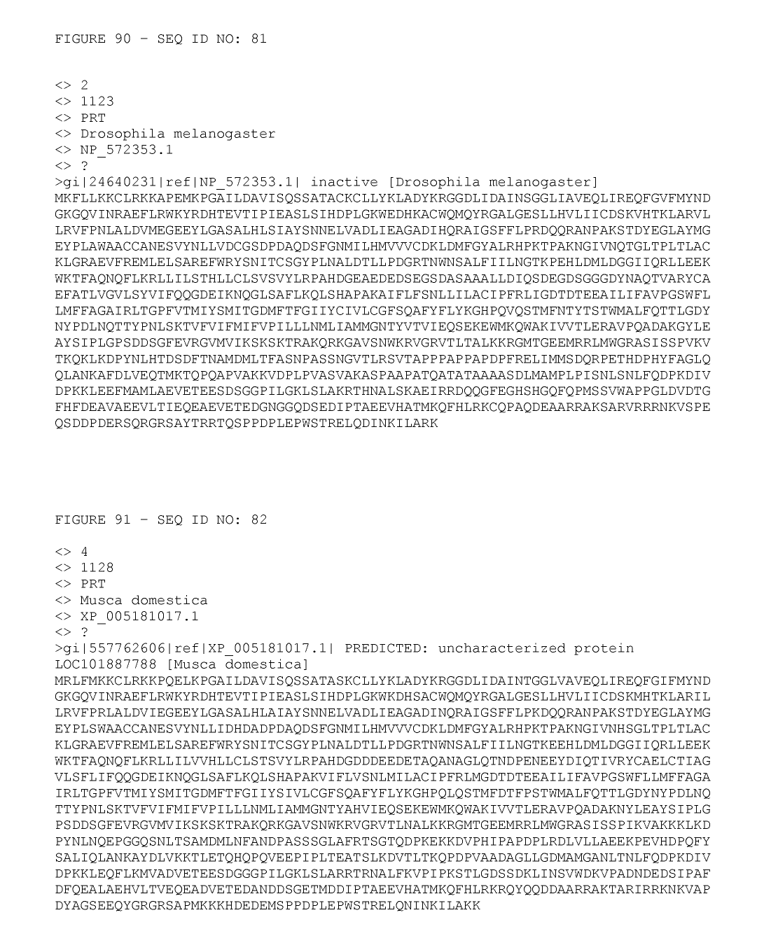

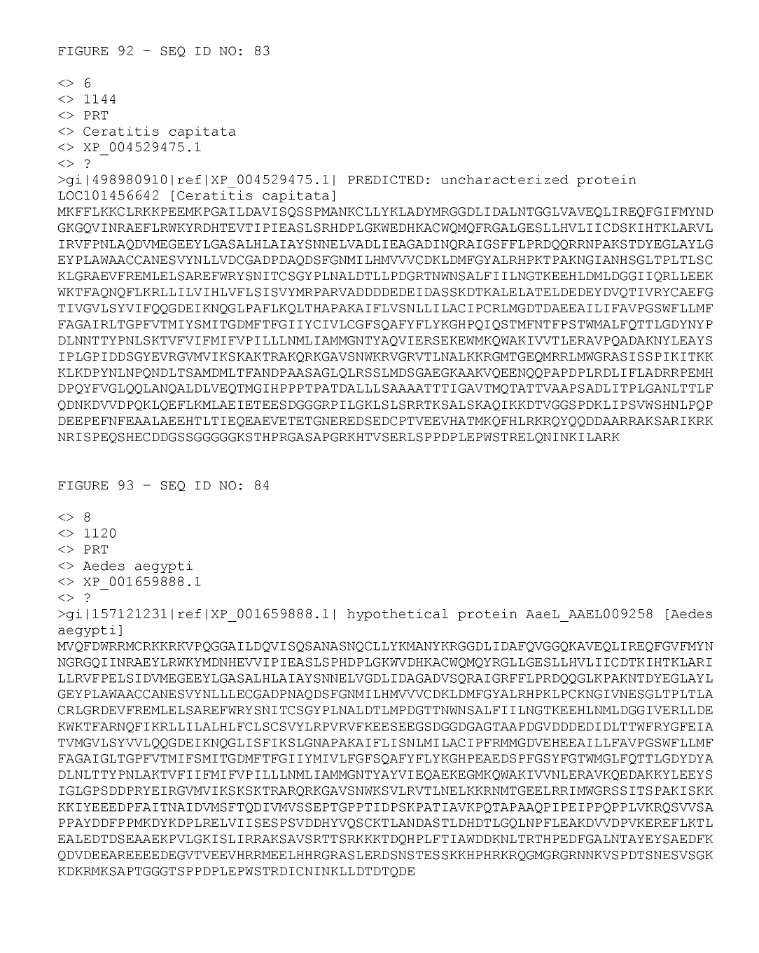

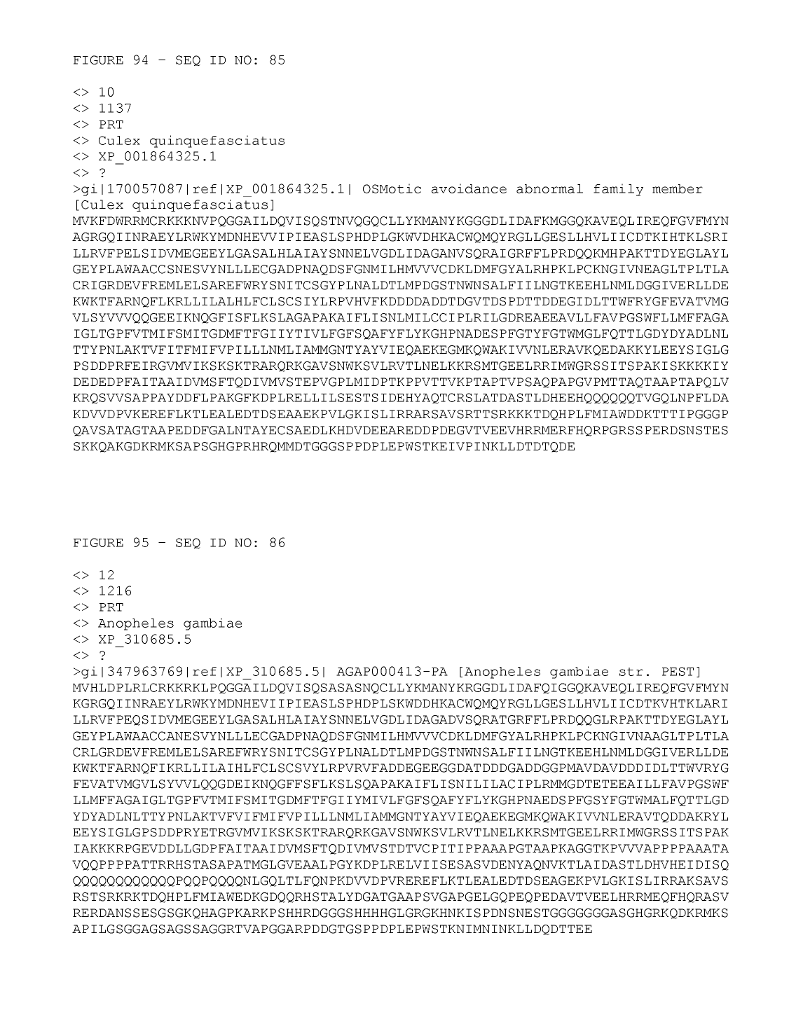

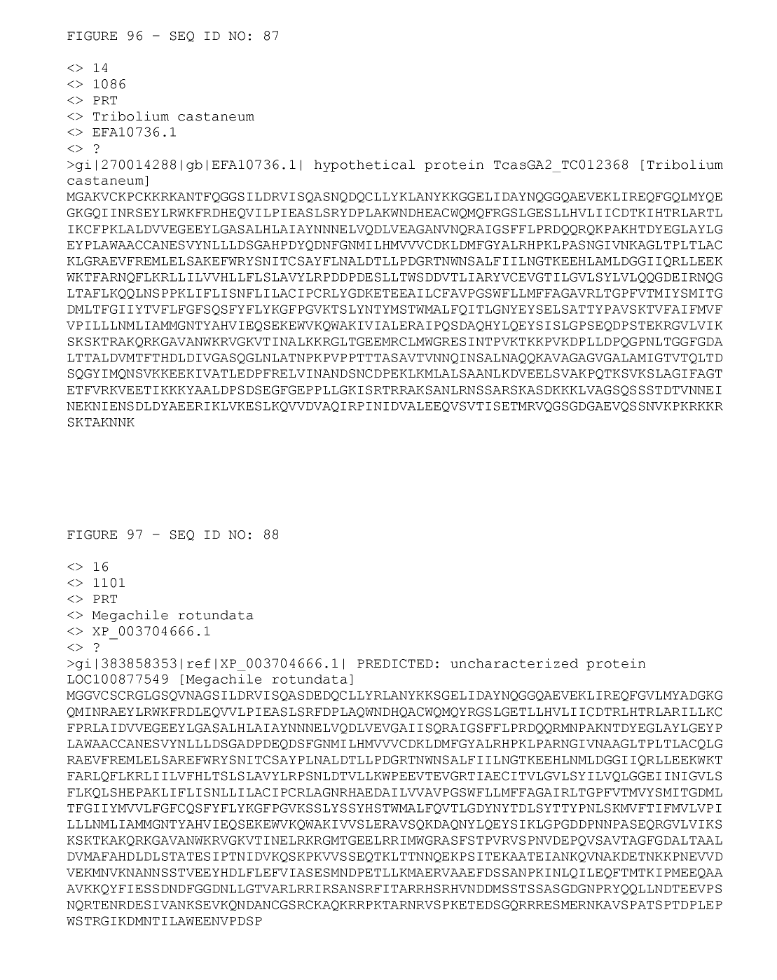

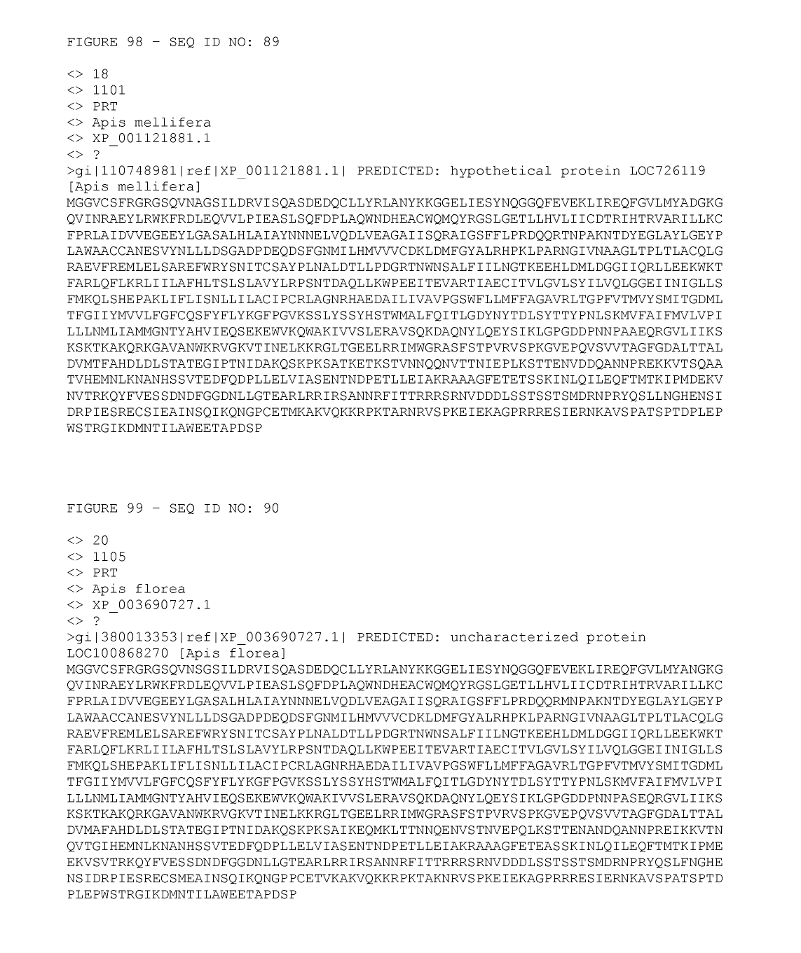

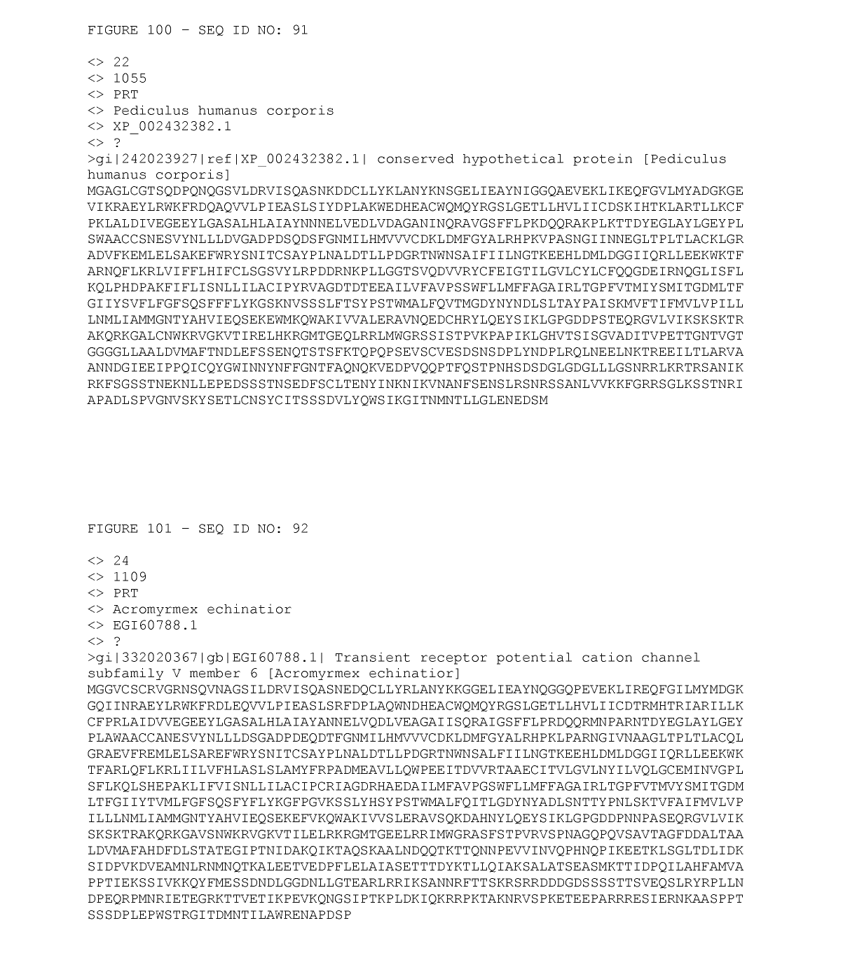

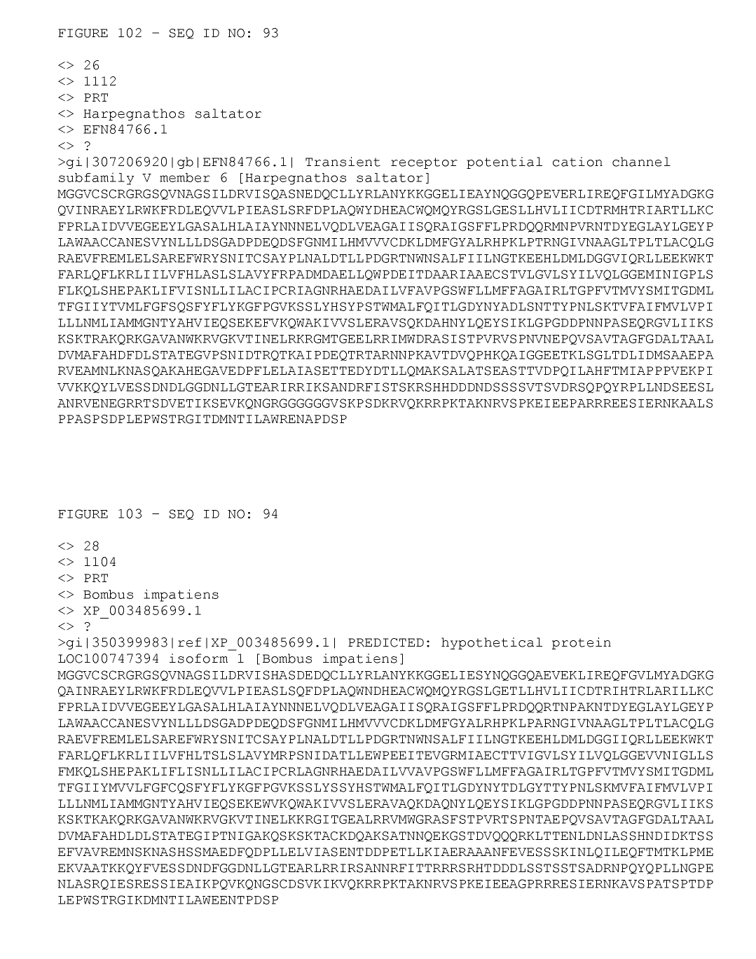

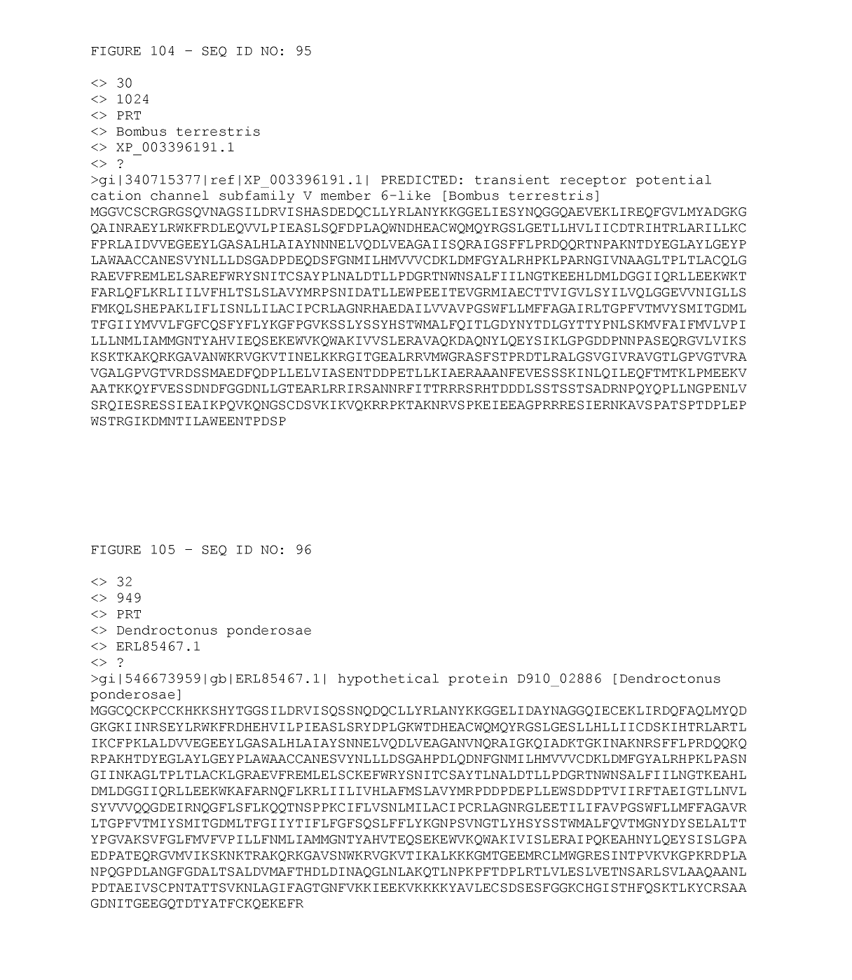

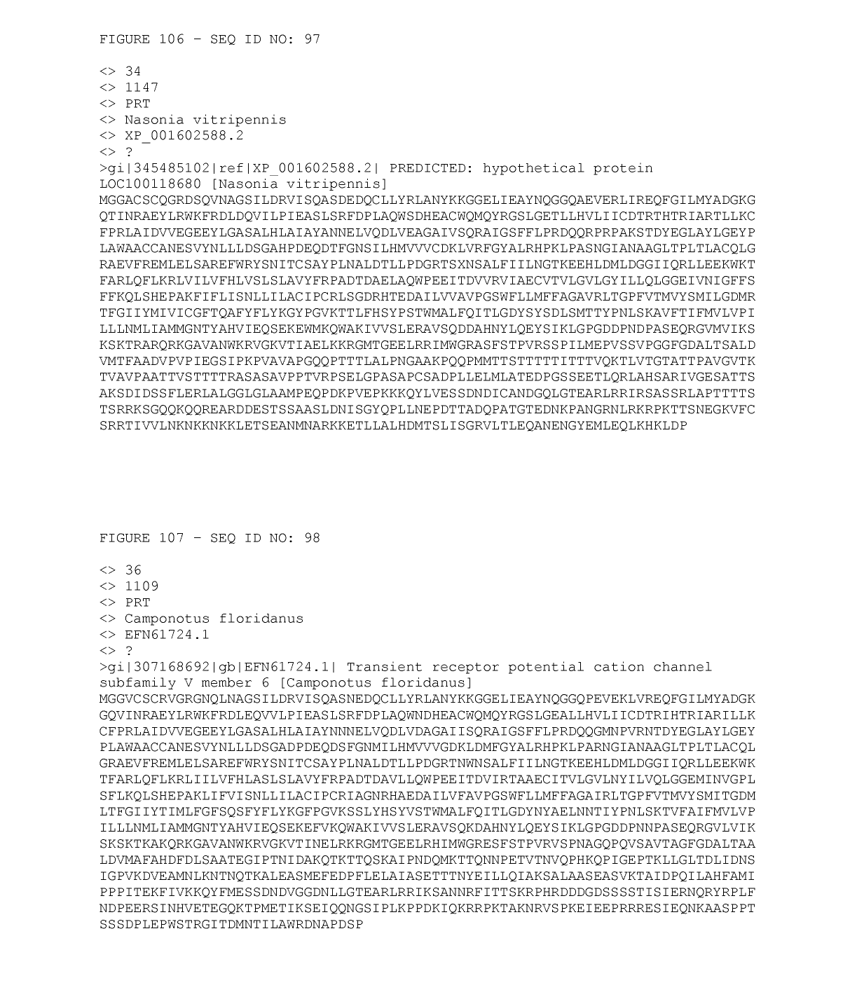

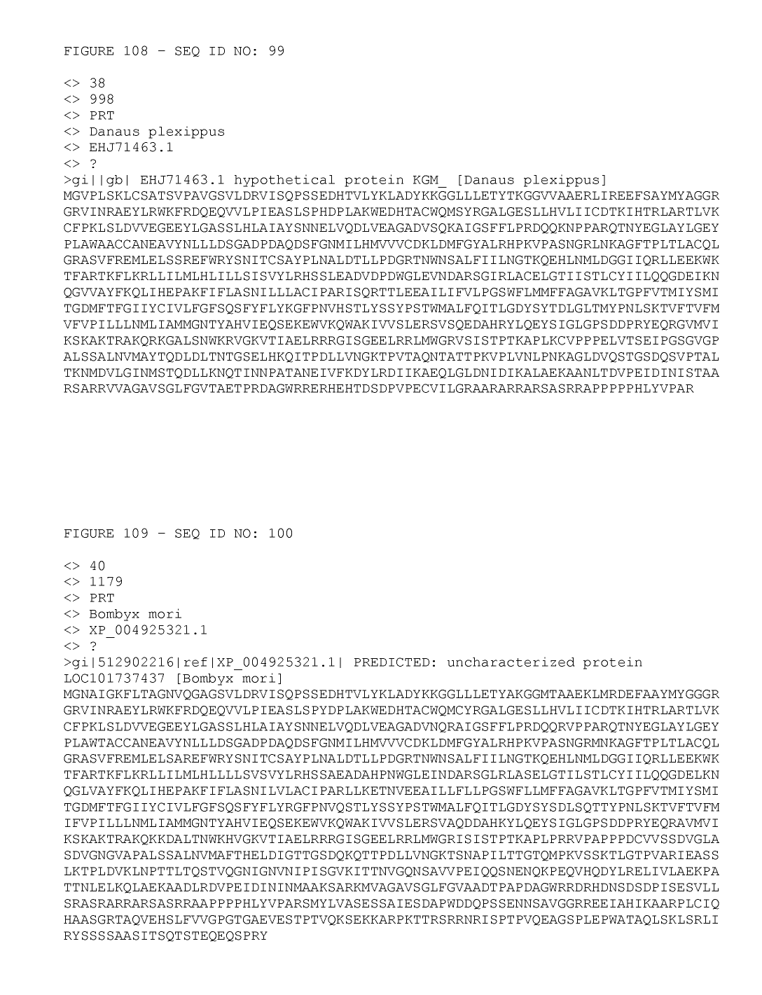

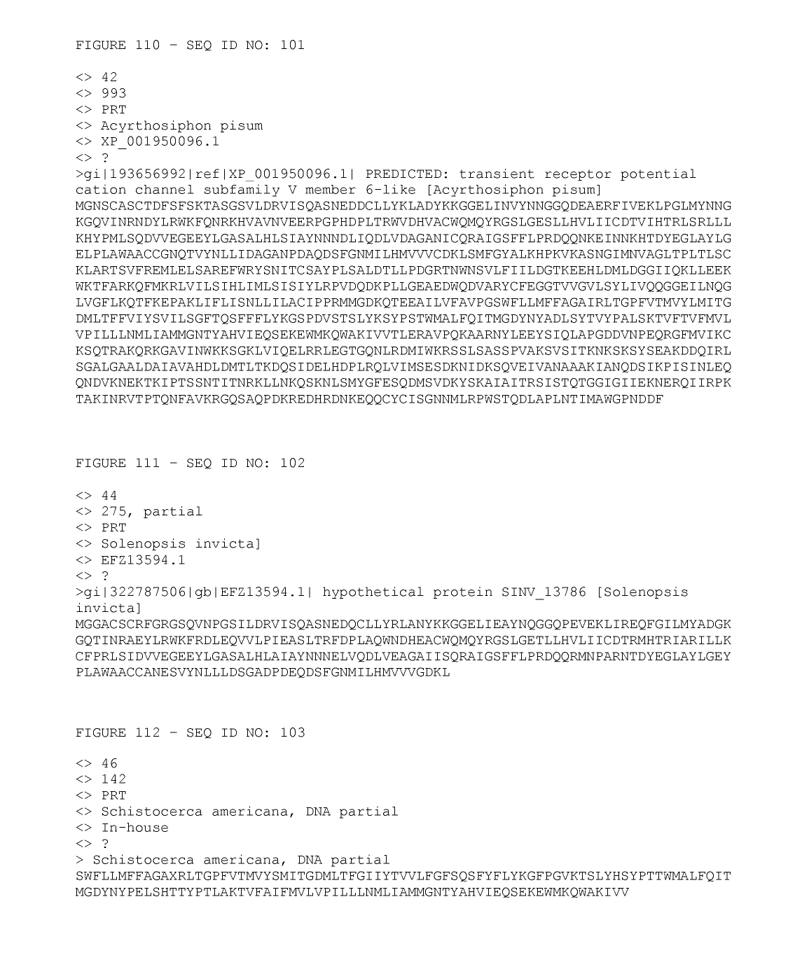

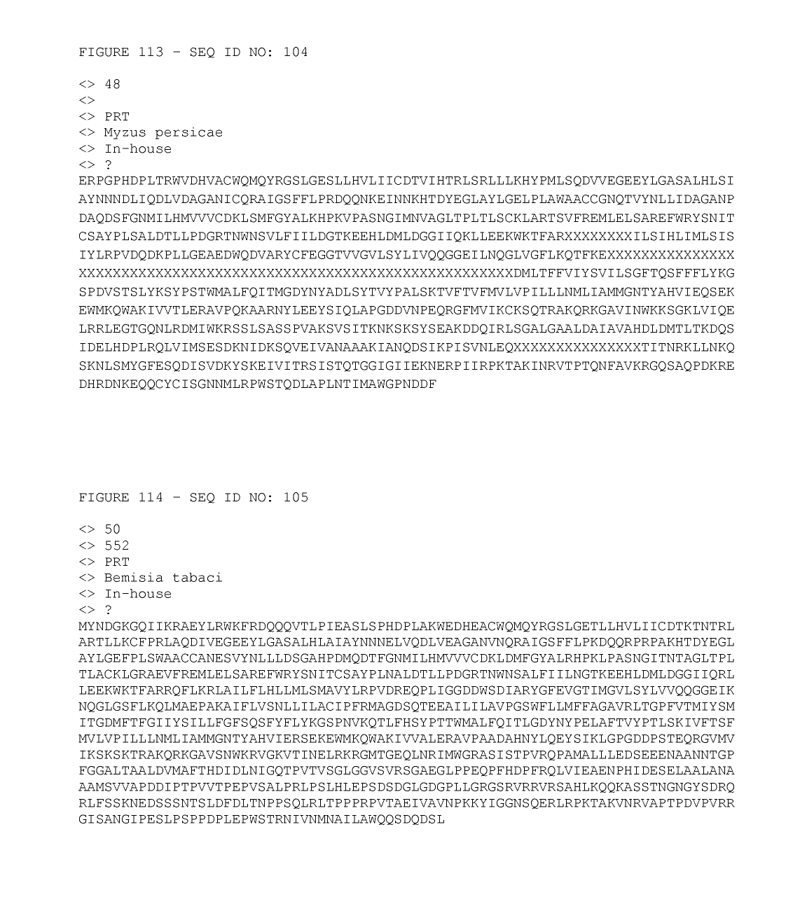

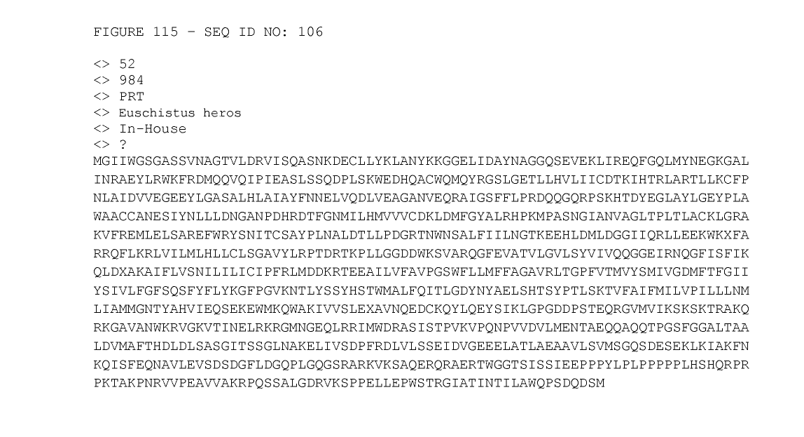

In certain embodiments, the Inactive protein sequences may be selected from the group consisting of a) a polypeptide comprising an amino acid sequence having a sequence selected from the group consisting of SEQ ID NOS: 81-106 (FIGS. 90-115); b) a polypeptide having at least 70% sequence identity to the SEQ ID NOS: 81-106 (FIGS. 90-115); and c) a fragment or a conservative variant of the polypeptide of a) or b).

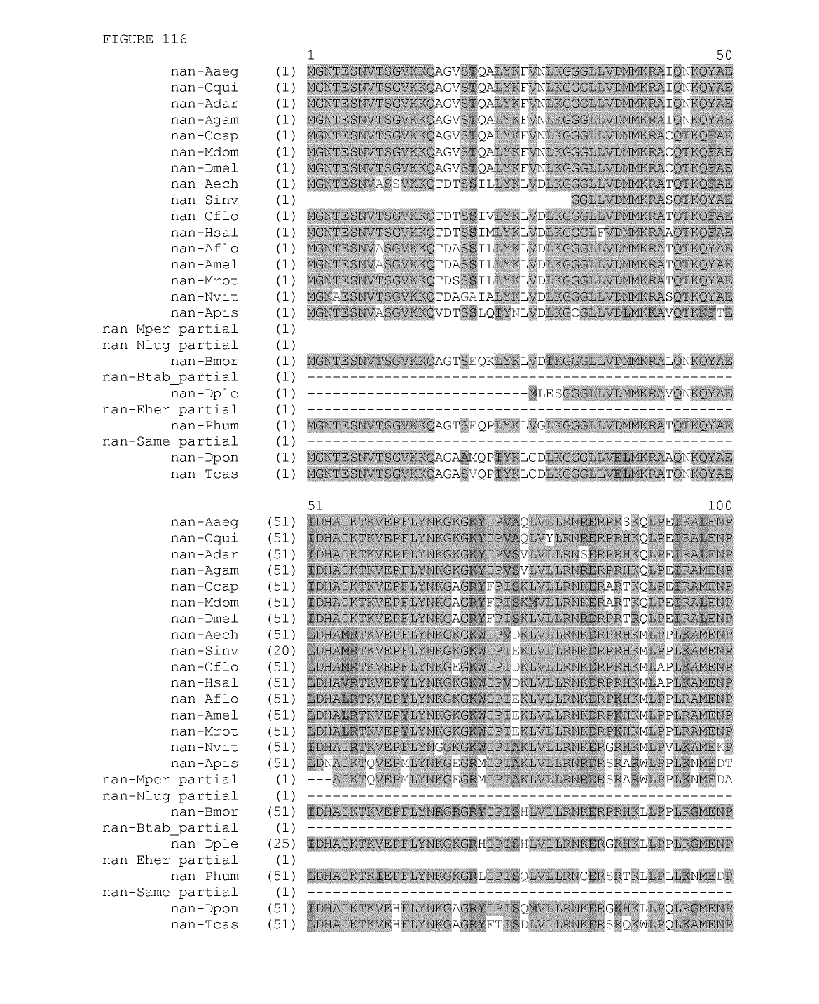

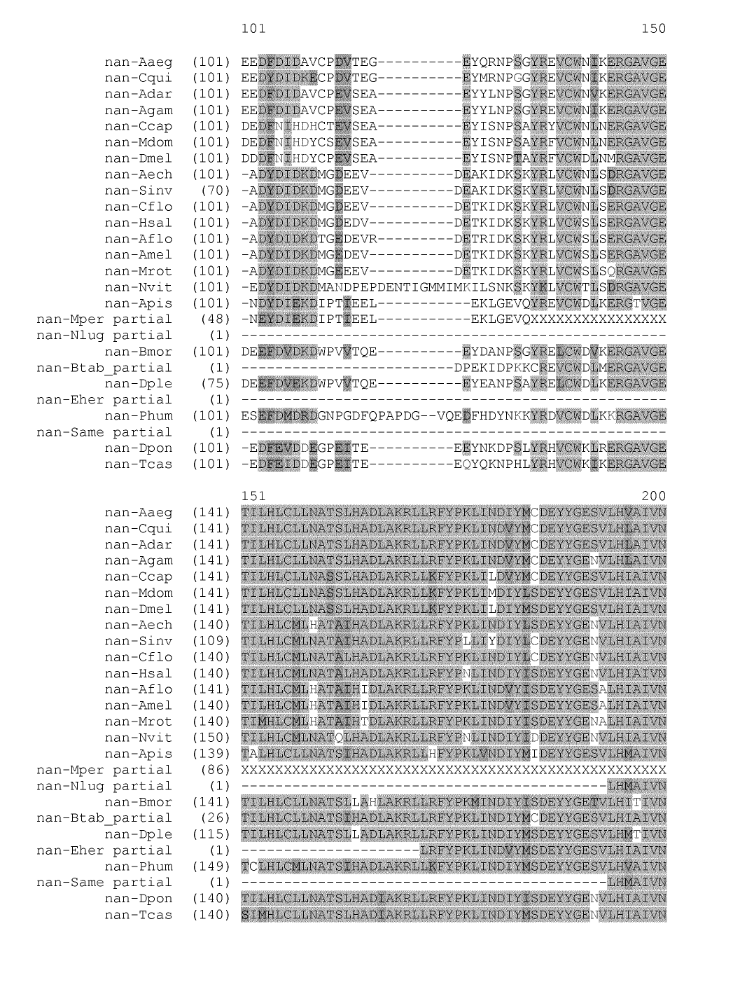

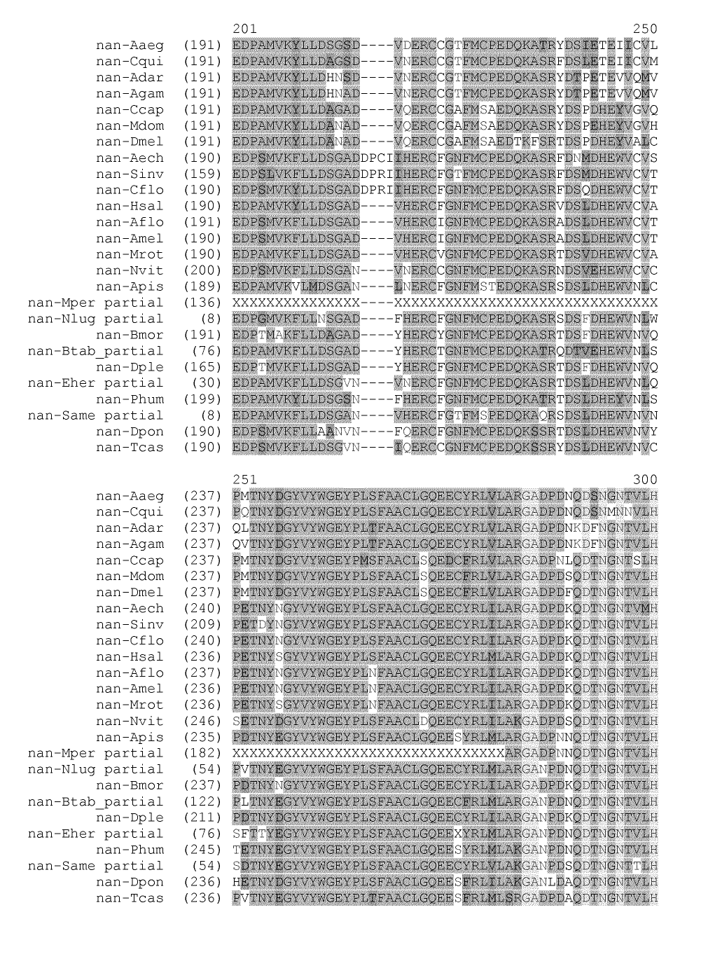

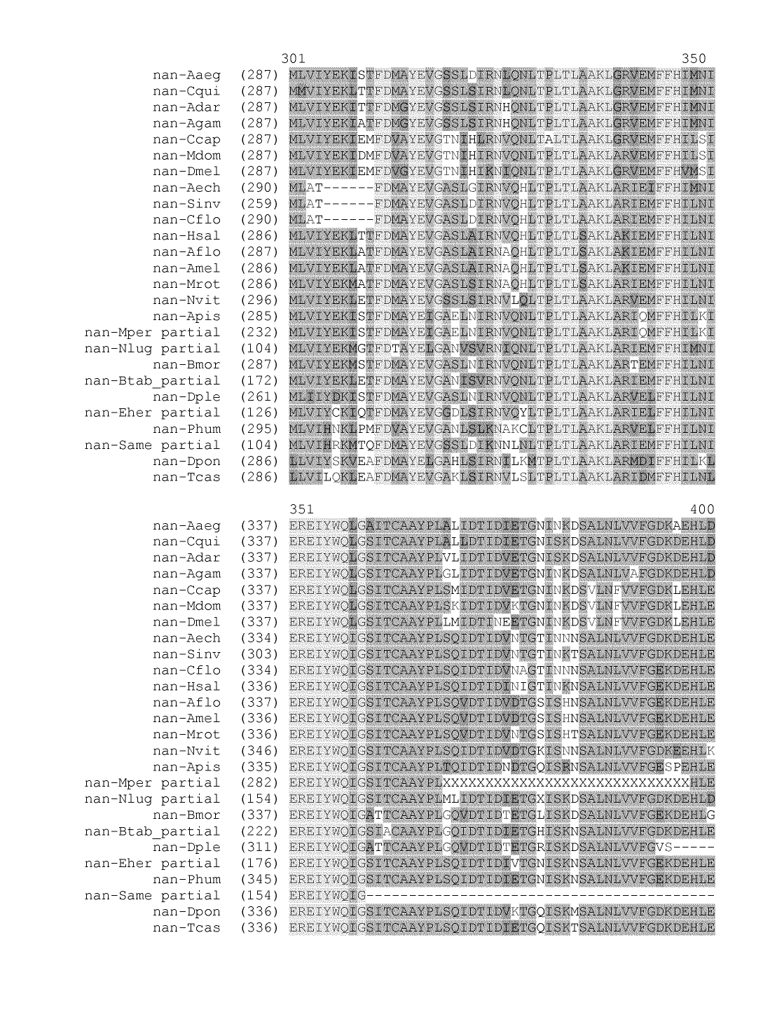

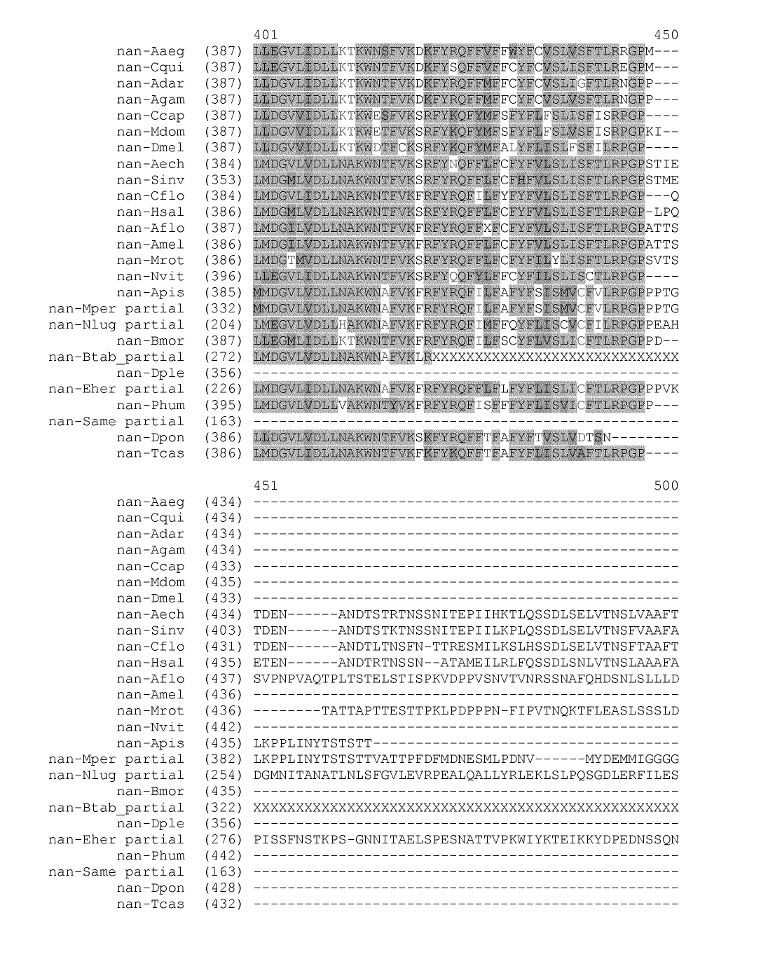

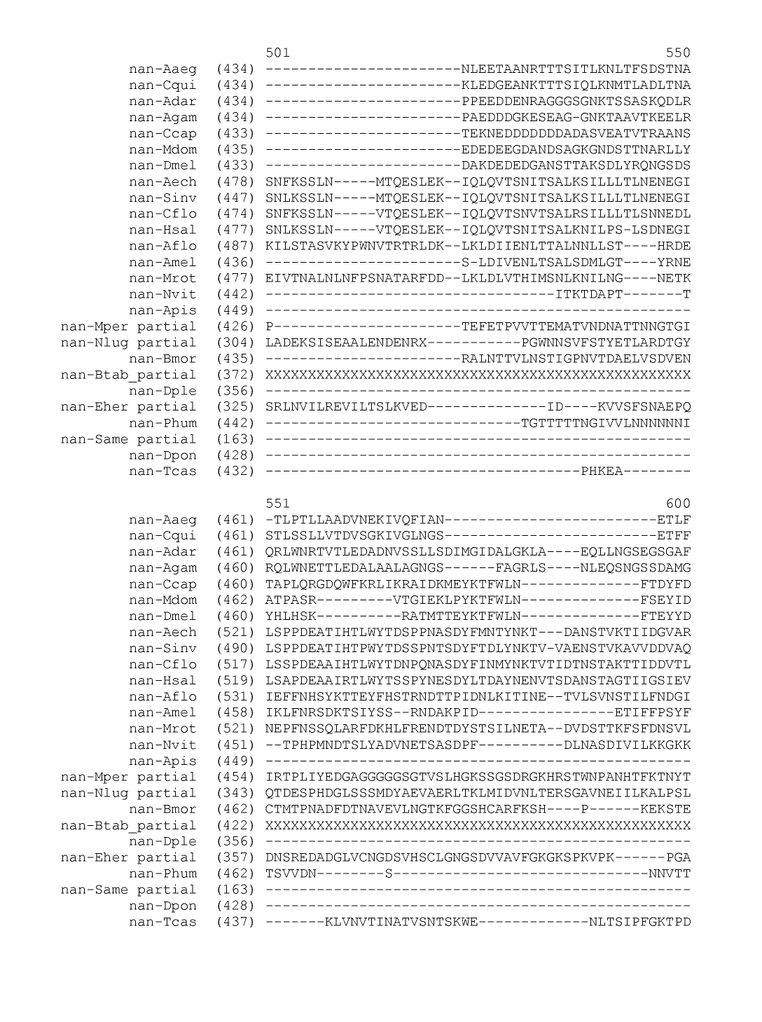

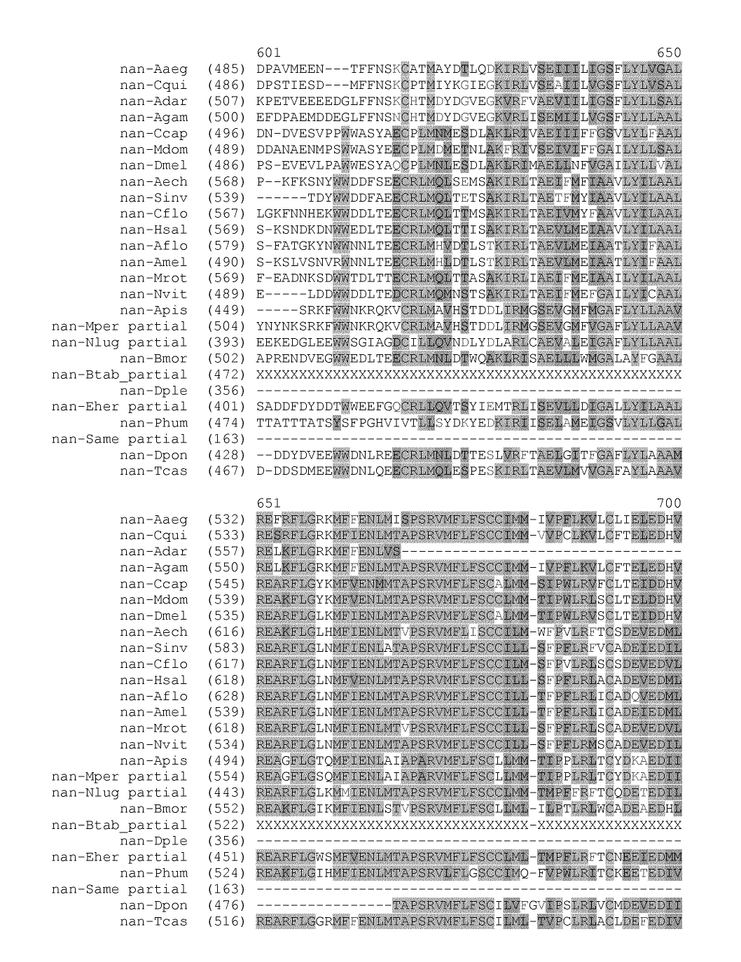

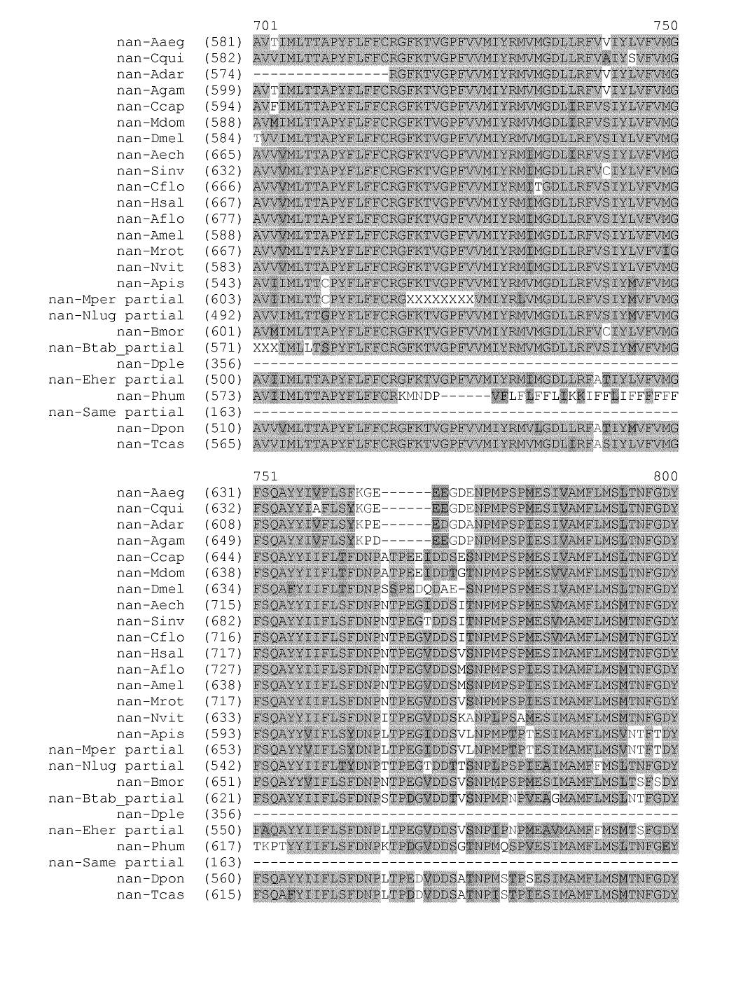

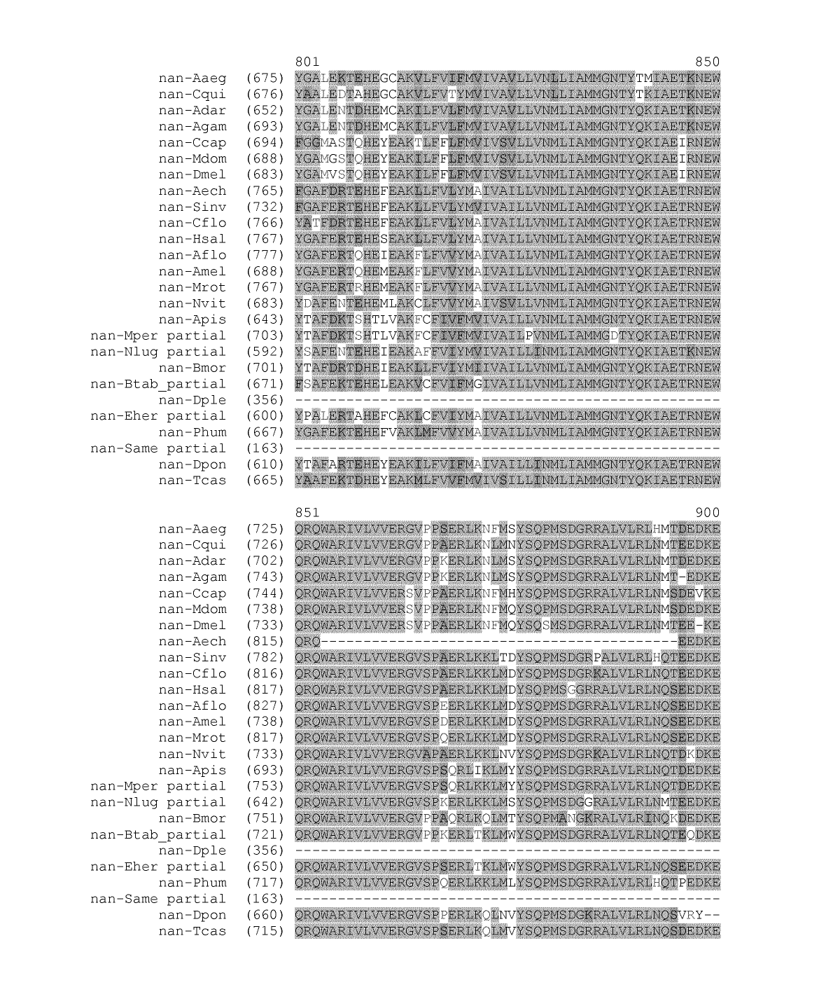

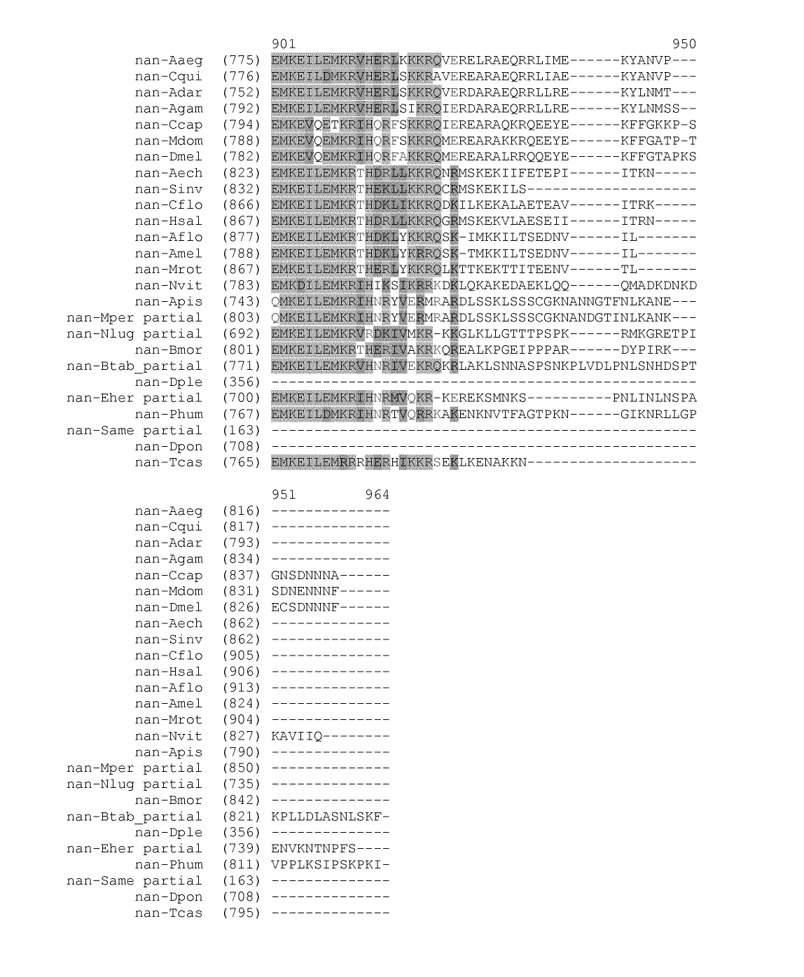

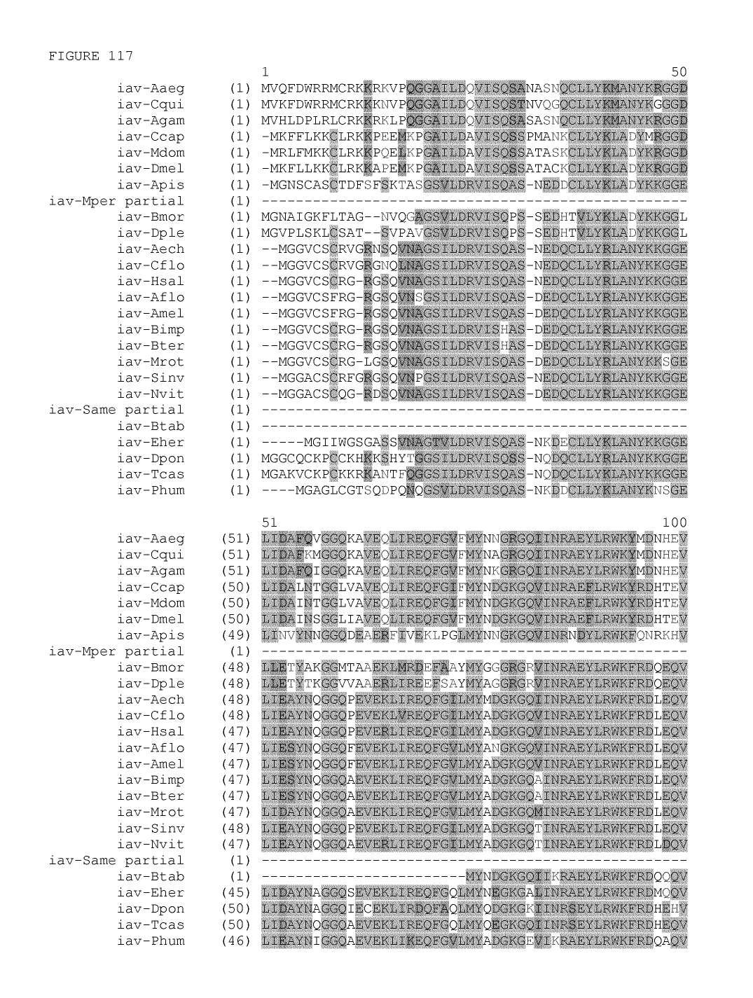

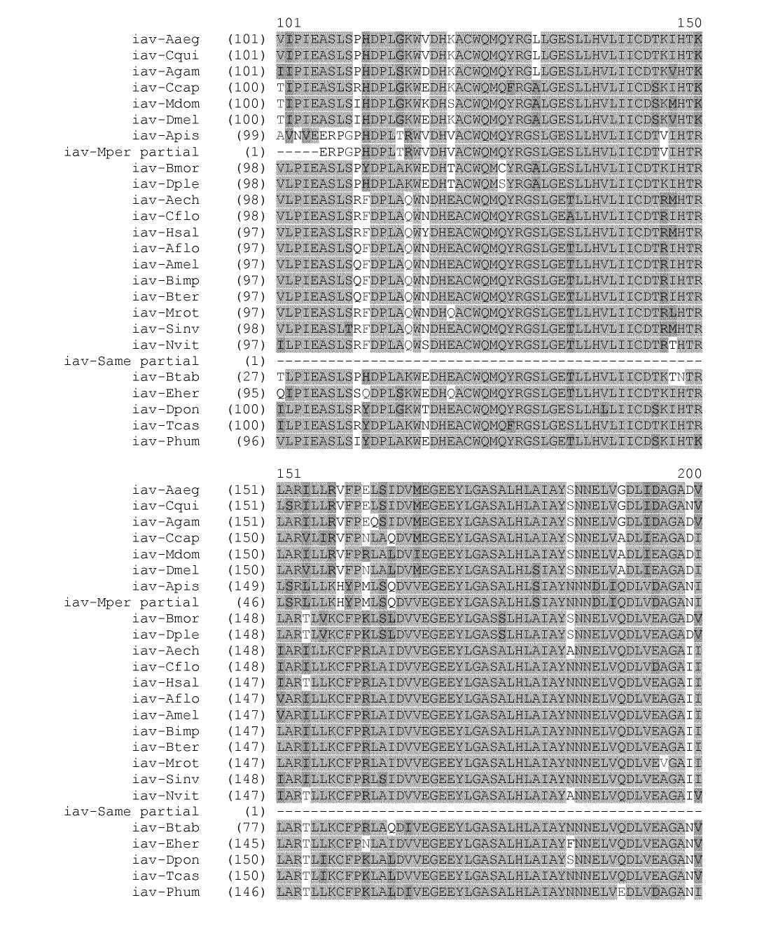

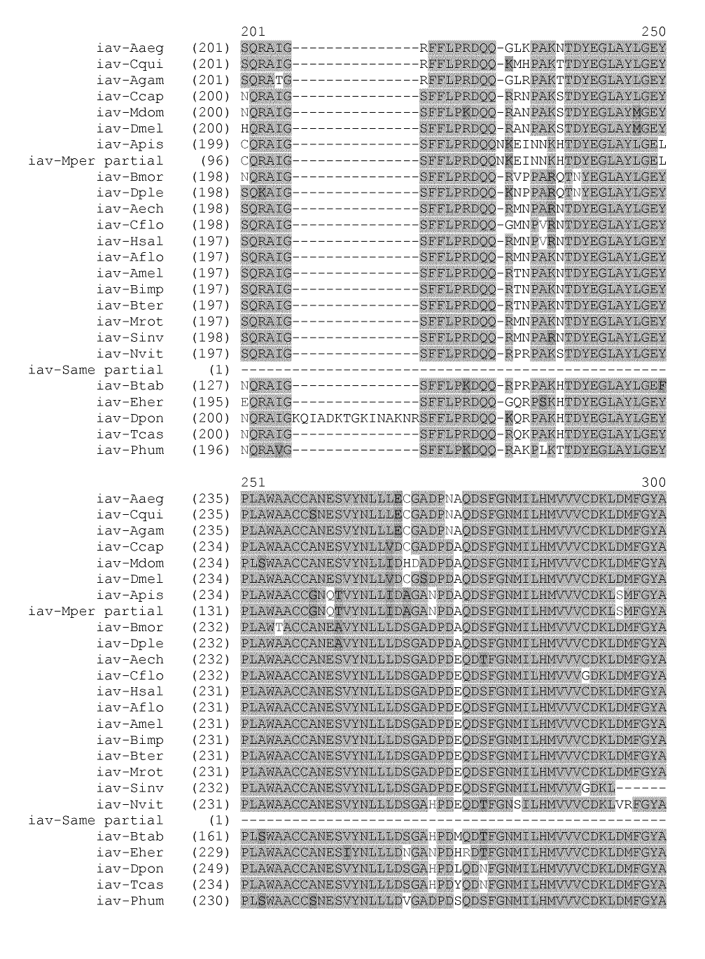

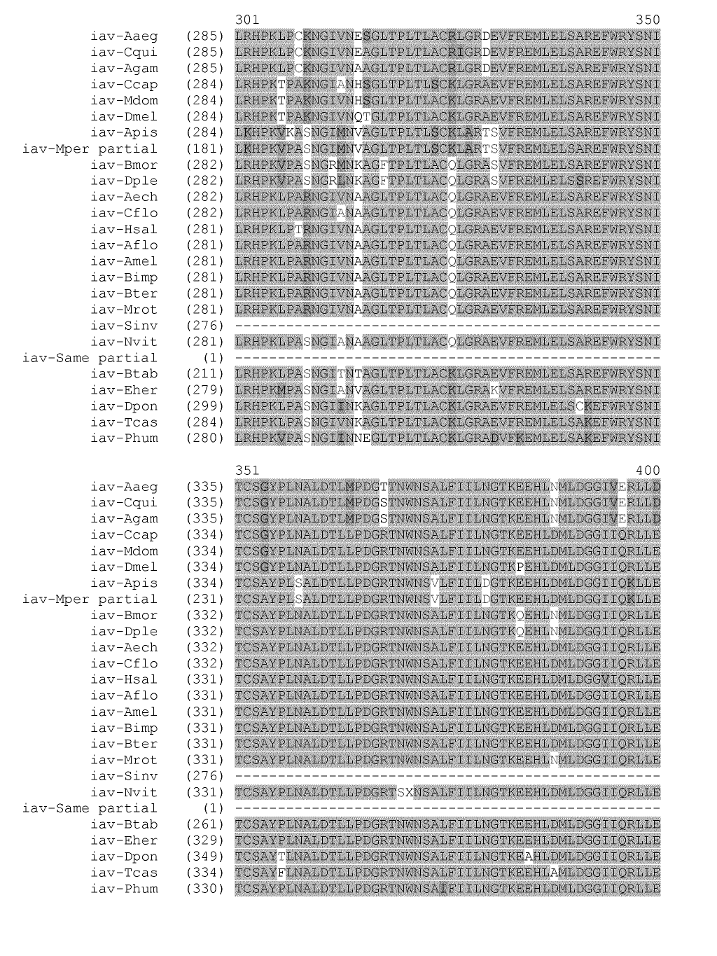

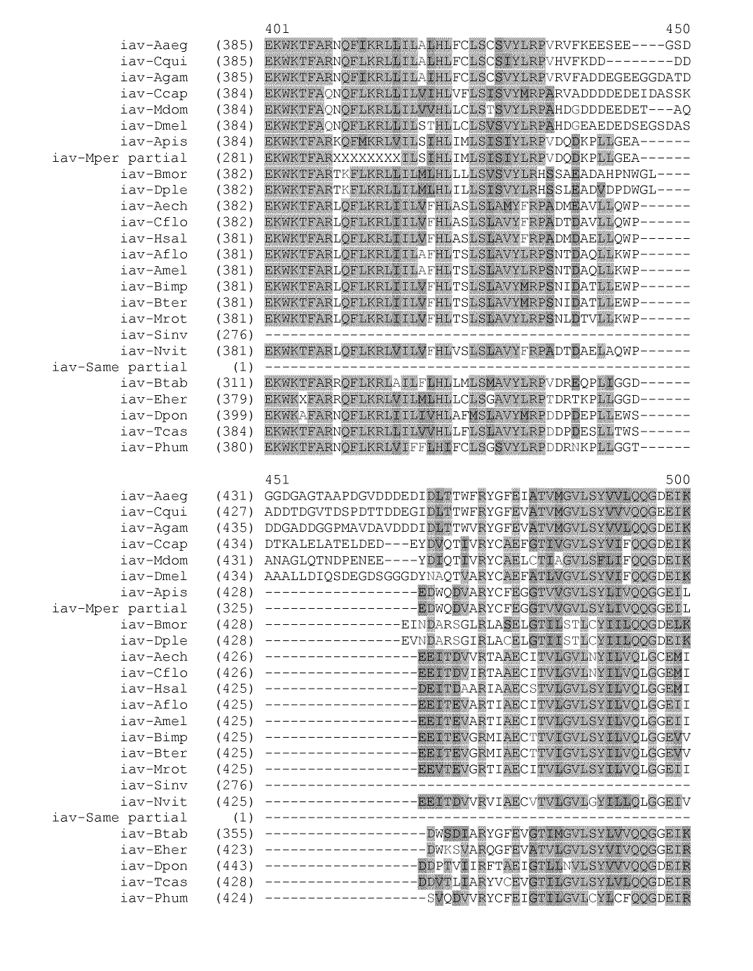

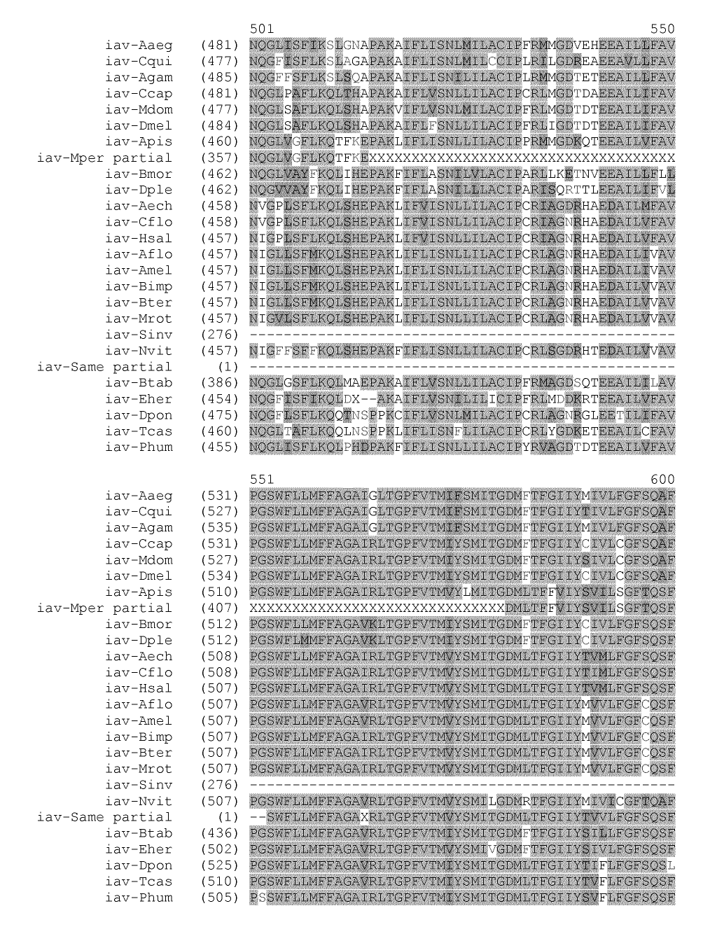

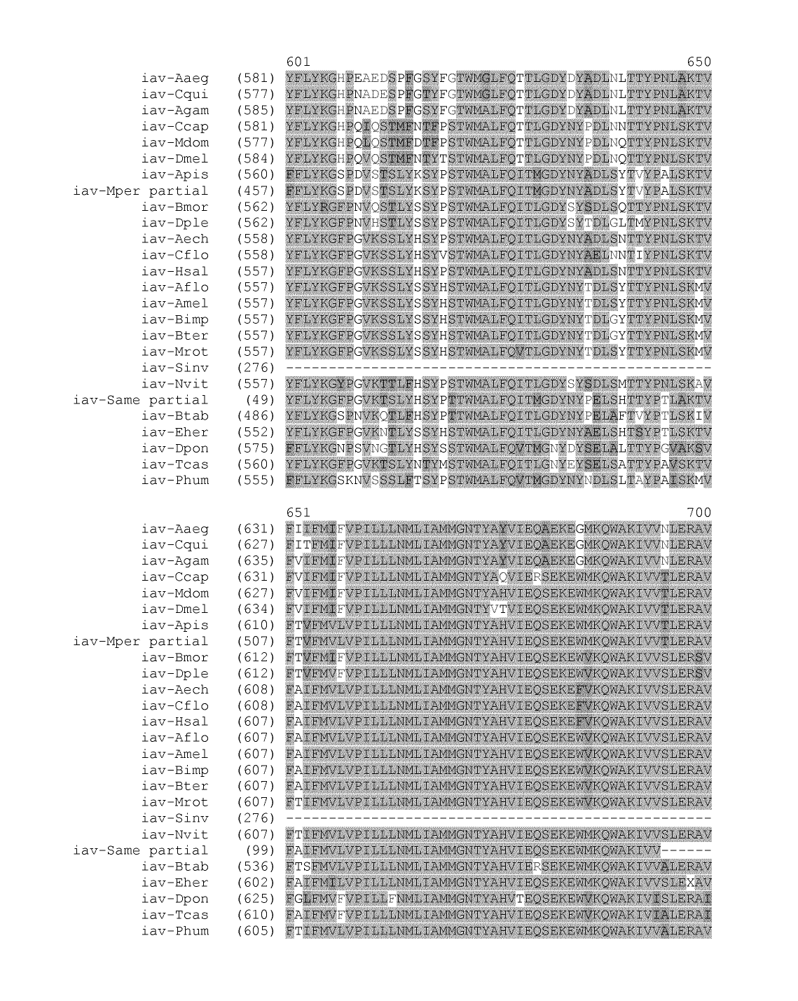

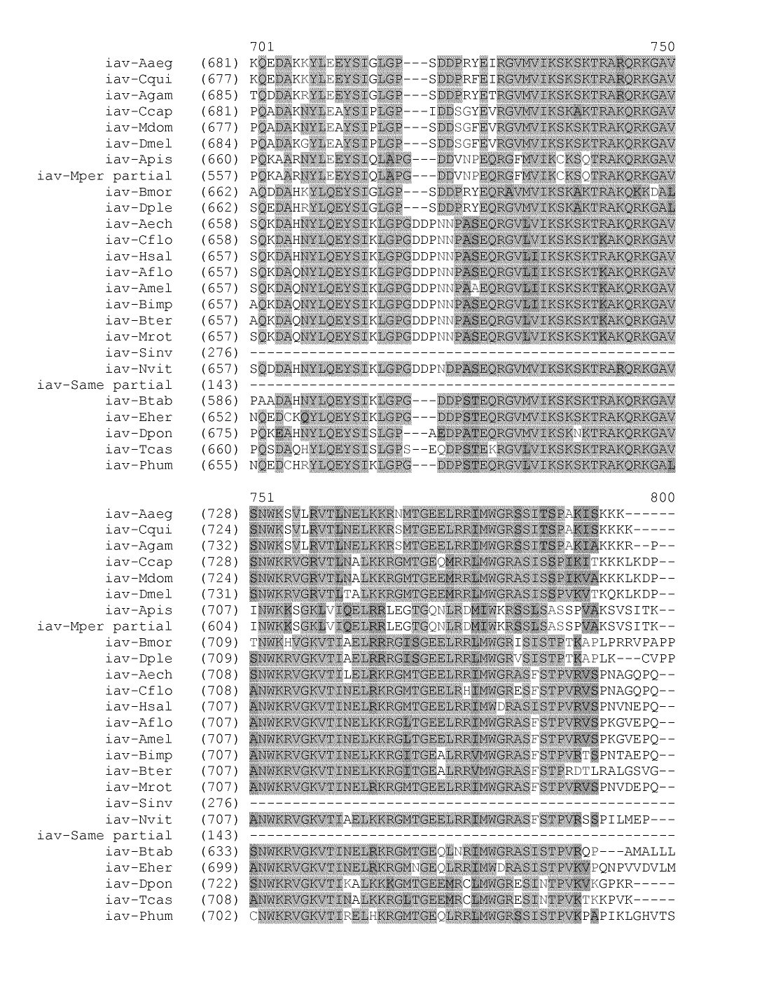

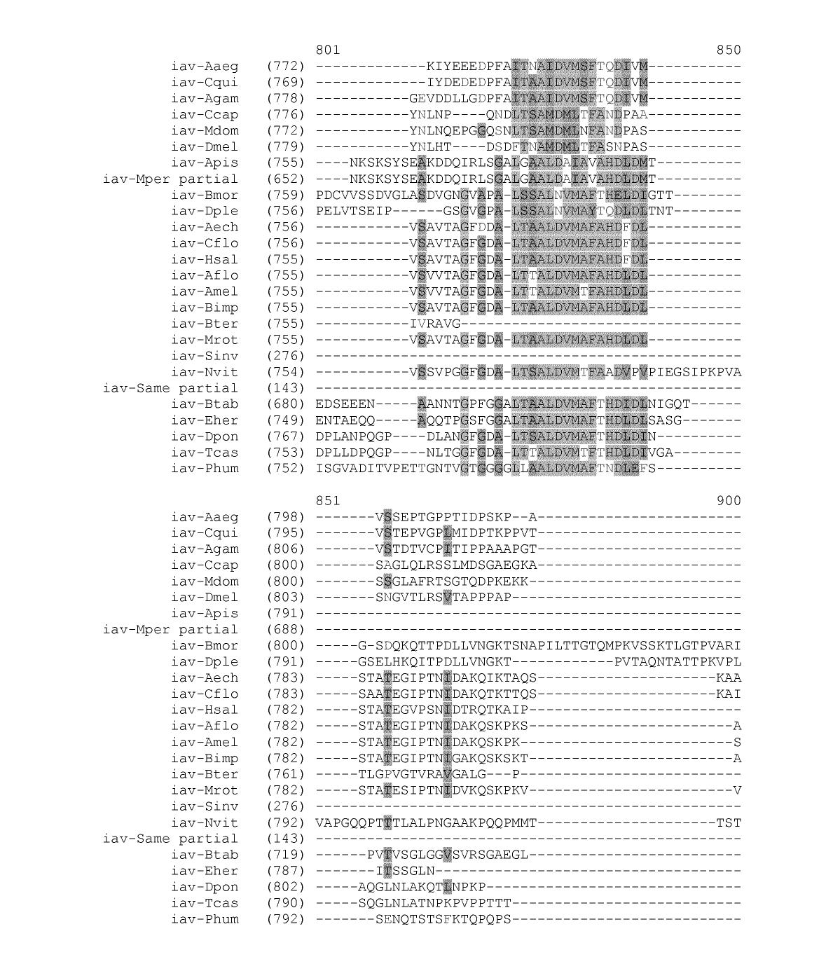

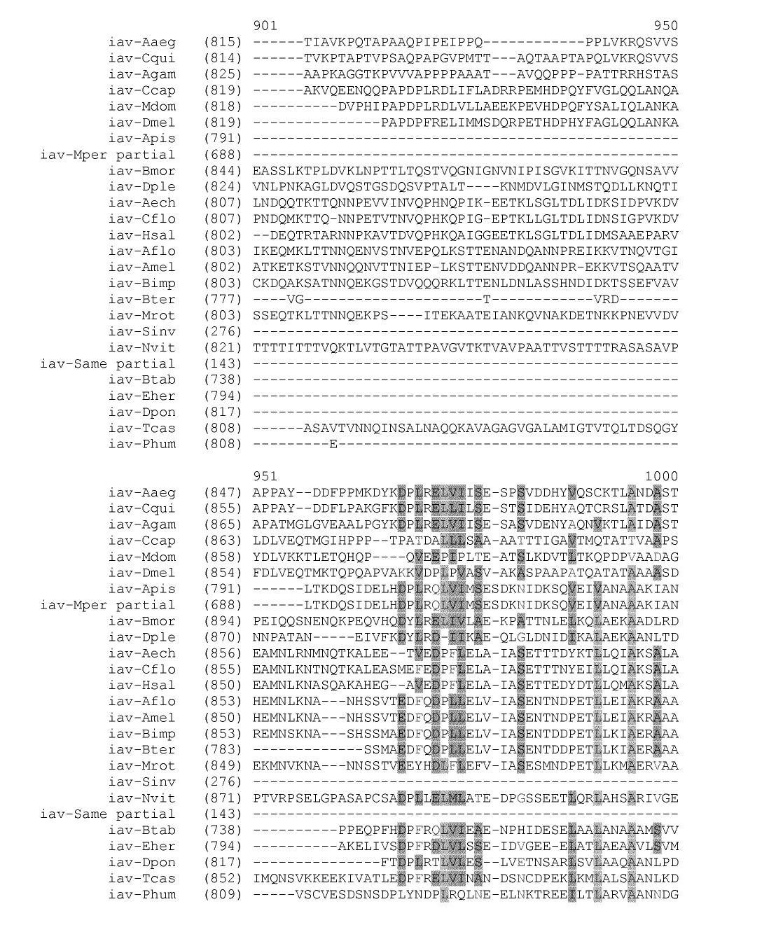

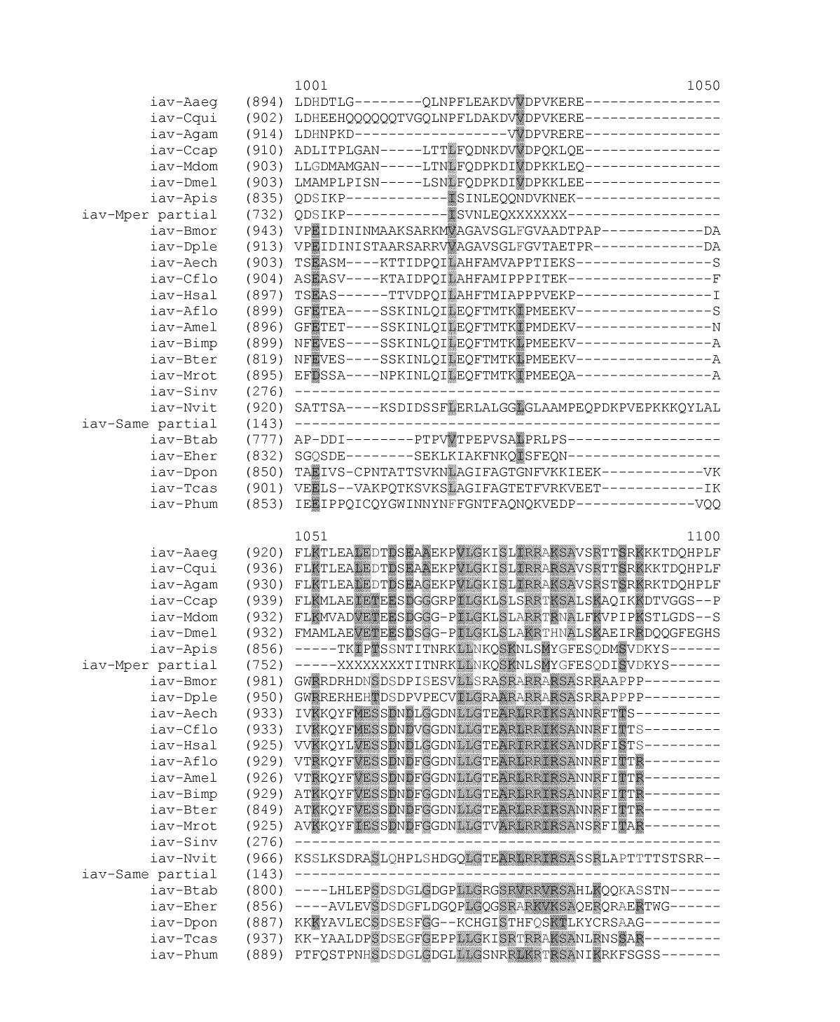

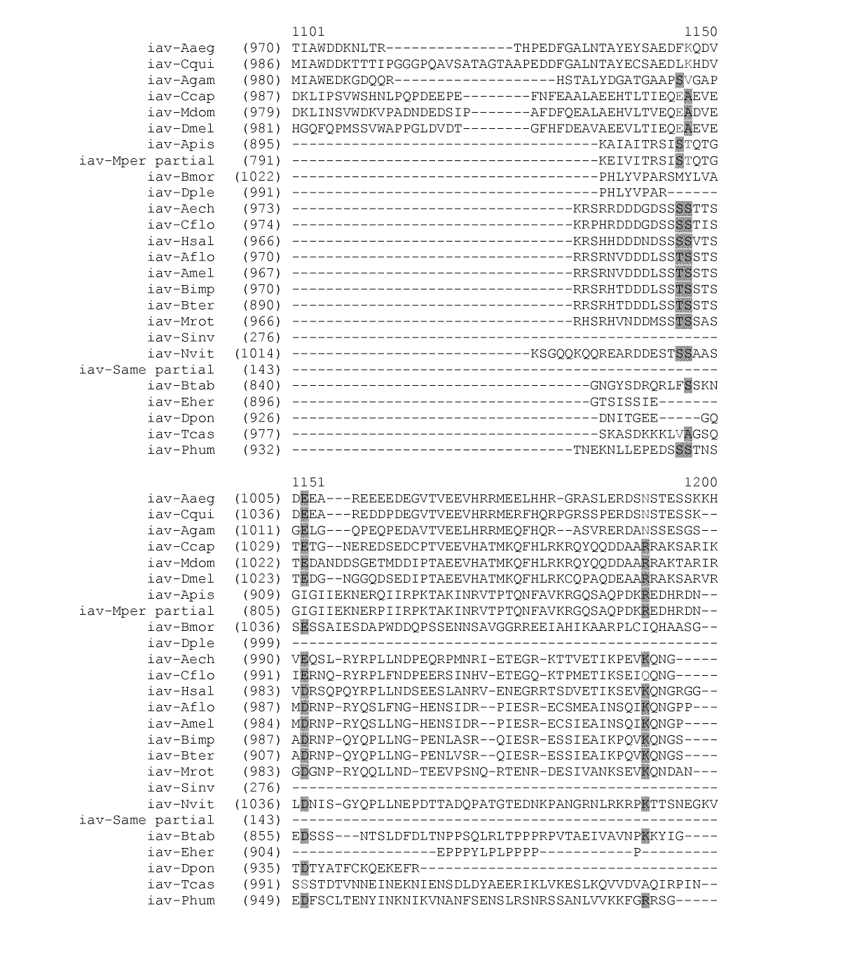

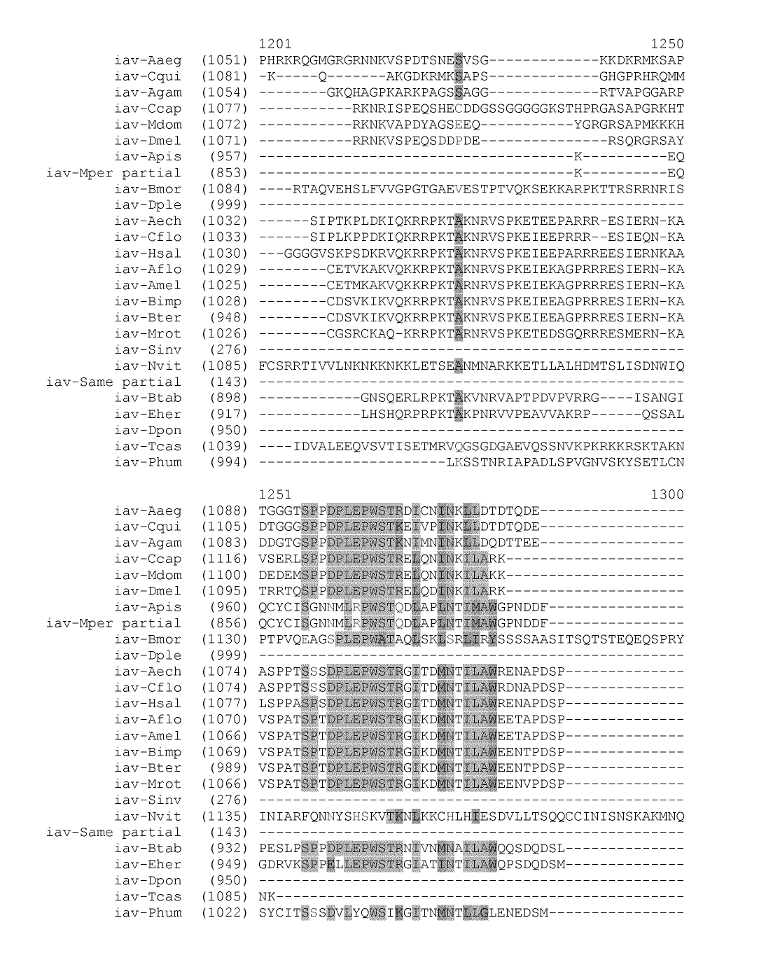

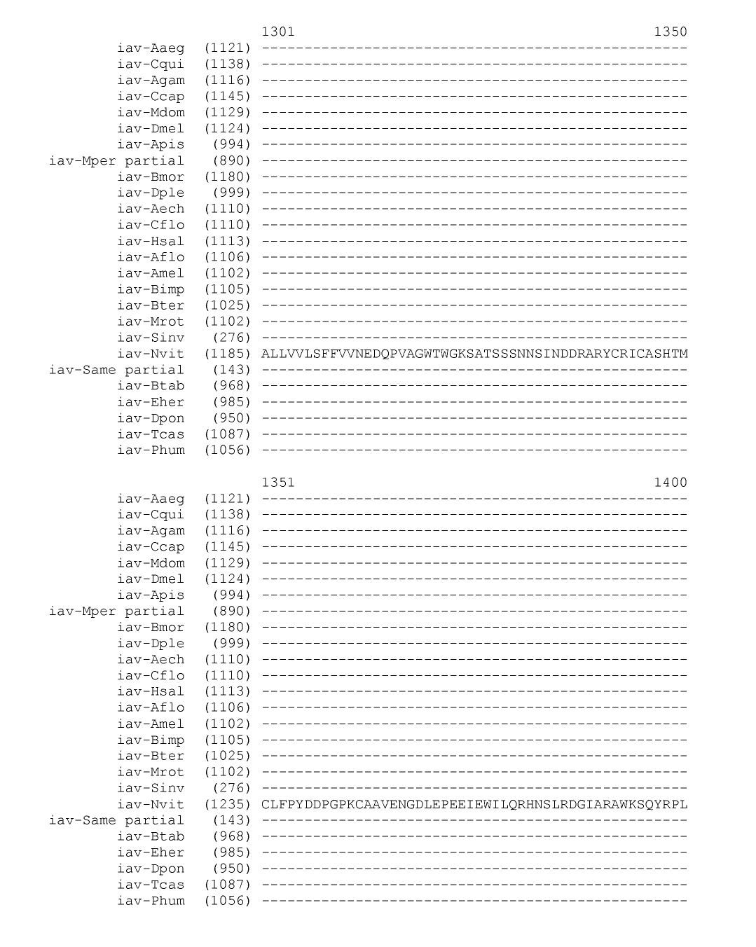

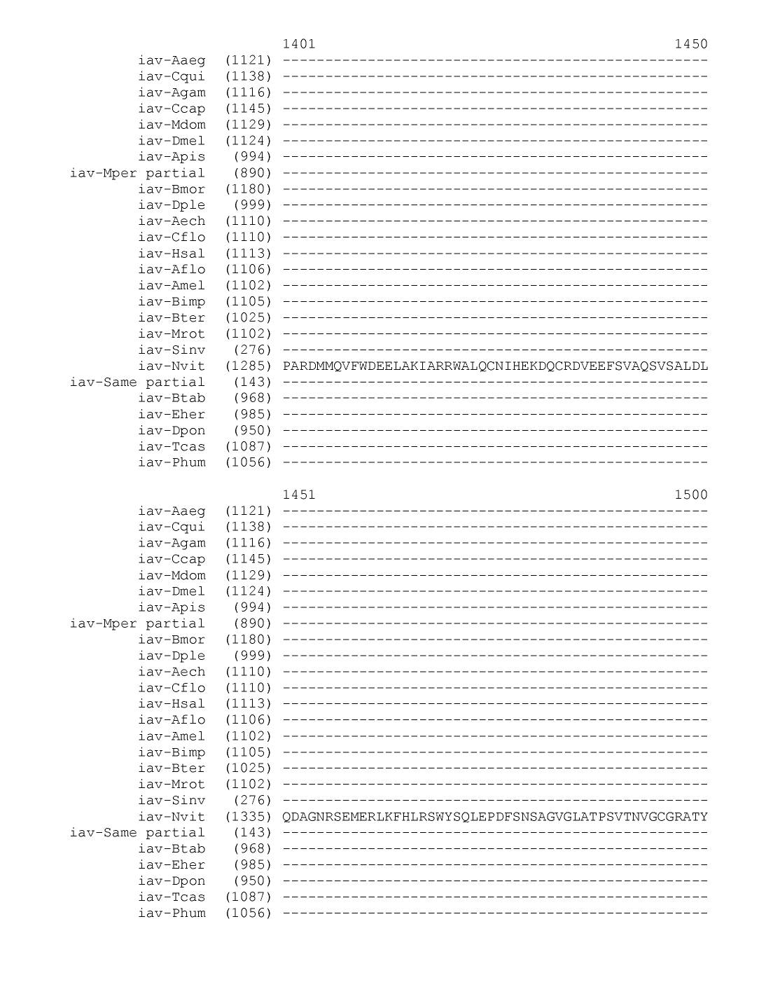

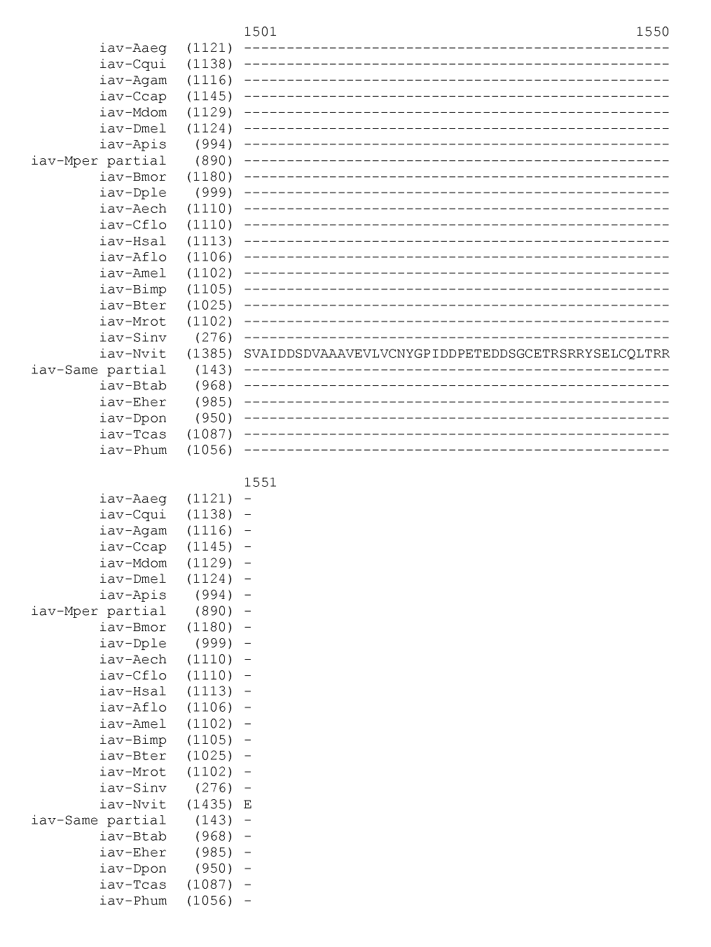

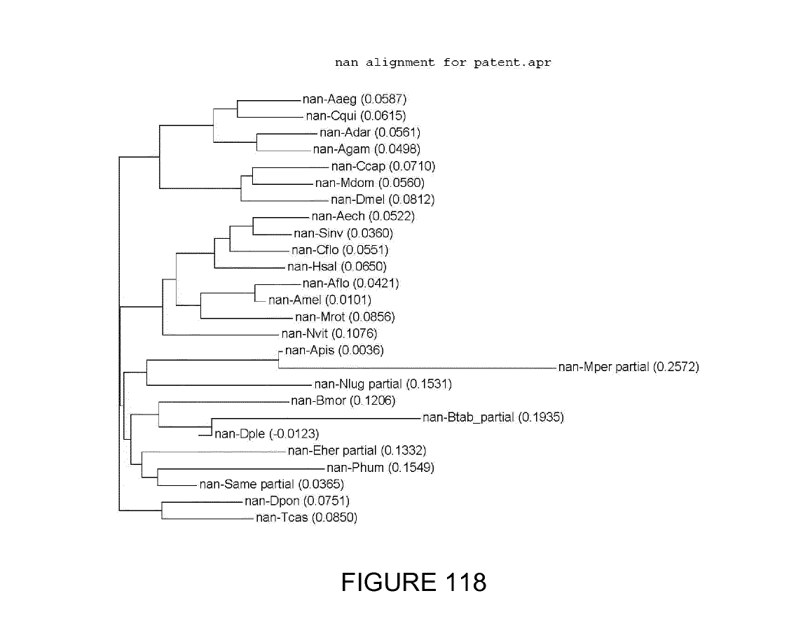

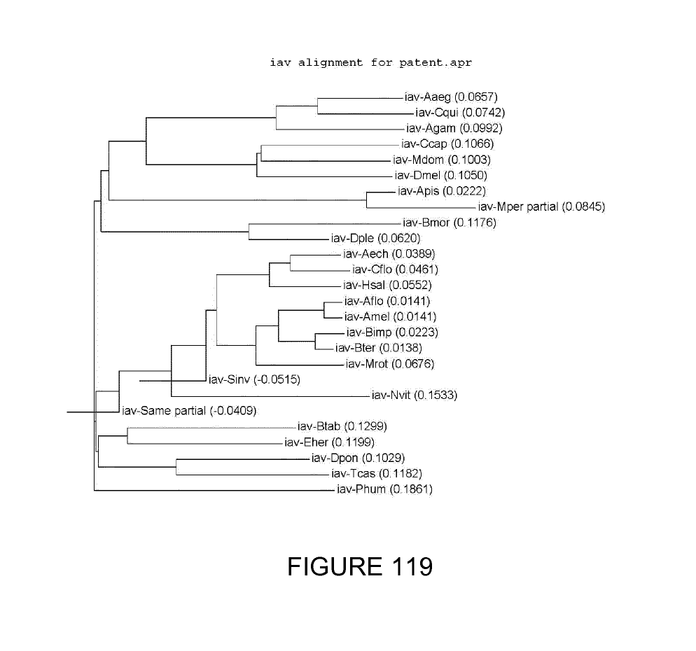

The sequence alignment of the Nanchung and Inactive proteins is shown in FIGS. 116 and 117, respectively. The family distribution tree for the Nanchung and Inactive proteins is shown in FIGS. 118 and 119, respectively.

As used herein, the “percent sequence identity” is determined by comparing two optimally aligned sequences over a comparison window, where the portion of the polynucleotide or a polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, divided by the number of matched positions by the total number of positions in the window of comparison and multiplying the result by 100 to yield the percentage of sequence identity. Alignment for the purposes of determining the percentage identity can be achieved in various ways that are within the skill in the art, for example, using publicly available computer software such as BLAST. Those skilled in the art can determine appropriate parameters for measuring alignment, including any algorithms needed to achieve optimal alignment over the full length of the sequences being compared.

As used herein, the term a “conservative variant” refers to an amino acid sequence in which a first amino acid is replaced by a second amino acid or amino acid analog having at least one similar biochemical property, which can be, for example, similar size, charge, hydrophobicity or hydrogen bonding capacity. For example, a first hydrophobic amino acid can be conservatively substituted with a second (non-identical) hydrophobic amino acid such as alanine, valine, leucine, or isoleucine, or an analog thereof. Similarly, a first basic amino acid can be conservatively substituted with a second (non-identical) basic amino acid such as arginine or lysine, or an analog thereof. In the same way, a first acidic amino acid can be conservatively substituted with a second (non-identical) acidic amino acid such as aspartic acid or glutamic acid, or an analog thereof or an aromatic amino acid such as phenylalanine can be conservatively substituted with a second aromatic amino acid or amino acid analog, for example tyrosine. In some embodiments, the peptide comprises conservative variant substitution of at least one amino acid, e.g. 1, 2, 3, 4, 5, 6, 7, 8, 9, 10 or more amino acids. Typically, a conservative variant will retain at least 50%, 55%, 60%, 65%, 70%, 75%, 80%, 85%, 90%, 95%, 97%, 98%, 99% or more of the activity of the wild-type peptide sequence.

Exemplary conservative variant substitution include, but are not limited to, replacement of Alanine (A) with D-ala, Gly, Aib, β-Ala, Acp, L-Cys, or D-Cys; Arginine (R) with D-Arg, Lys, D-Lys, homo-Arg, D-homo-Arg, Met, Be, D-Met, or D-Ile; Asparagine (N) with D-Asn, Asp, D-Asp, Glu, D-Glu, Gln, or D-Gln; Aspartic acid (D) with D-Asp, D-Asn, Asn, Glu, D-Glu, Gln, or D-Gln; Cysteine (C) with D-Cys, S-Me-Cys, Met, D-Met, Thr, or D-Thr; Glutamine (Q) with D-Gln, Asn, D-Asn, Glu, D-Glu, Asp, or D-Asp; Glutamic Acid (E) with D-Glu, D-Asp, Asp, Asn, D-Asn, Gln, or D-Gln; Glycine (G) with Ala, D-Ala, Pro, D-Pro, Aib, β-Ala, or Acp; Isoleucine (I) with D-Ile, Val, D-Val, AdaA, AdaG, Leu, D-Leu, Met, or D-Met; Leucine (L) with D-Leu, Val, D-Val, AdaA, AdaG, Leu, D-Leu, Met, or D-Met; Lysine (K) with D-Lys, Arg, D-Arg, homo-Arg, D-homo-Arg, Met, D-Met, Ile, D-Ile, Orn, or D-Orn; Methionine (M) with D-Met, S-Me-Cys, Be, D-Ile, Leu, D-Leu, Val, or D-Val; Phenylalanine (F) with D-Phe, Tyr, D-Thr, L-Dopa, His, D-His, Trp, D-Trp, Trans-3,4 or 5-phenylproline, AdaA, AdaG, cis-3, 4 or 5-phenylproline, Bpa, or D-Bpa; Proline (P) with D-Pro, L-I-thiazolidine-4-carboxylic acid, or D- or -L-1-oxazolidine-4-carboxylic acid (U.S. Pat. No. 4,511,390); Serine (S) with D-Ser, Thr, D-Thr, allo-Thr, Met, D-Met, Met (O), D-Met (O), L-Cys, or D-Cys; Threonine (T) with D-Thr, Ser, D-Ser, allo-Thr, Met, D-Met, Met (O), D-Met (O), Val, or D-Val; Tyrosine (Y) with D-Tyr, Phe, D-Phe, L-Dopa, His, or D-His; and Valine (V) with D-Val, Leu, D-Leu, Ile, D-Ile, Met, D-Met, AdaA, or AdaG.

Conservative variants of the insect or mammalian TRPV channels can be prepared according to methods for altering peptide sequences known in the art, and include those that may be found in references, which compile such methods, e.g., Molecular Cloning: A Laboratory Manual, J. Sambrook, et al., eds., Second Edition, Cold Spring Harbor, or Current Protocols in Molecular Biology, F. M. Ausubel, et al., eds., John Wiley & Sons, Inc. New York.

Conservative variants of TRPV and/or TRP channel may also be made by alteration of a nucleic acid encoding the TRPV and/or TRP polypeptide.

In certain embodiments, the screening method of the present invention may be a high-throughput screening. High-throughput screening (HTS) refers to a method for scientific experimentation that uses robotics, data processing and control software, liquid handling devices, and sensitive detectors. High-Throughput Screening or HTS allows a researcher to quickly conduct millions of biochemical, genetic or pharmacological tests. HTS is well known in the art, including, for example, U.S. Pat. Nos. 5,976,813; 6,472,144; 6,692,856; 6,824,982; and 7,091,048.

HTS uses automation to run a screen of an assay against a library of candidate compounds. An assay is a test for specific activity: usually inhibition or stimulation of a biochemical or biological mechanism. Specifically, an assay would be screening for inhibition or stimulation of the insect TRPV channel. Typical HTS screening libraries or “decks” can contain from 100,000 to more than 2,000,000 compounds.

The key labware or testing vessel of HTS is the microtiter plate, which is a small container, usually disposable and made of plastic that features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, a researcher fills each well of the plate with the appropriate reagents that he or she wishes to conduct the experiment with. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as current or voltage measurements, colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

### IV. Formulations, Applications and Uses

In another aspect, the invention provides a compound selected by the screening assay described herein. It is to be understood that analogs, derivatives, isomers, and pharmaceutically acceptable salts of the compounds selected by the screening assays described herein as well as any formulations including the selected compound are also included herein.

The compound or group of compounds being determined or identified by the method according to the invention can be used in methods of insect control, e.g., by modulating insect feeding behavior. The identified compounds, analogs, derivatives, isomers, and pharmaceutically acceptable salts thereof are also referred to as “active agents” or “active ingredients” herein.

In some embodiments, the compound modulates the feeding behavior of an insect. As used herein, the term “feeding behavior” refers to the process by which organisms, such as insects obtain food. Without being bound by theory, the modulation of an insect TRPV elicits a signaling pathway that brings forth motor neuron modulation which may decrease feeding behavior of an insect, and such modulation of an insect TRPV channel in an insect may lead to a decrease in feeding behavior of such insect leading to the insect's death by starvation. Thus, again without being bound by theory, it is believed that compounds that modulate (e.g., activate or inhibit) an insect TRPV channel may be used as insecticides. This decrease in feeding behavior can be used to destroy insects in a particular location and thus control such insects. Thus, in some embodiments, the method comprises modulation of TRPV ion channel or family members in the insect with a compound identified by a screening method described herein.

Accordingly, in certain embodiments, the invention provides a method of insect control by modulating feeding behavior in an insect using a compound identified by the screening methods described herein. As used in context of methods of insect control, compounds identified by the screening methods described herein also include analogs, derivatives, isomers and pharmaceutically acceptable salts of such compounds.

In one embodiment, the methods and active agents described herein are applicable to insects that are agricultural or horticultural pest.

Examples of agricultural pests include, but are not limited to, Aphids (including Aphis fabae, Aphis gossypii, Aphis pomi, Aulacorthum solani, Brevicoryne brassicae, Dysaphis plantaginea, Macrosiphum euphorbiae, Macrosiphum euphorbiae, Macrosiphum rosae, Myzus persicae, Nasonovia ribisnigri); Whiteflies (including Aleurodes spp., Bemisia spp., Dialeurodes spp., Trialeurodes spp.); Planthoppers (including Laodelphax striatellus, Nilaparvata lugens, Siphanta spp., Sogatella furcifera); Leafhoppers (including Amrasca spp., Empoasca spp.); Scales (including Aspidiotus spp., Chrysomphalus aonidum, Icerya purchase, Unaspis citri); Mealybugs (including Maconellicoccus spp., Paracoccus spp., Planococcus spp.); Pollen beetles (including Carpophilus spp., Meligethes spp.); Thrips (including Caliothrips spp., Frankliniella spp., Scirtothrips spp., Thrips spp.); and Psyllids (including Bactericera spp., Cacopsylla spp., Diaphorina citri, Paratrioza cockerelli).

For example, vegetable and cole crops are sensitive to infestation by one or more of the following insect pests: aphids, brown plant hopper, alfalfa looper, armyworm, beet armyworm, artichoke plume moth, cabbage budworm, cabbage looper, cabbage webworm, corn earworm, celery leafeater, cross-striped cabbagewon, european corn borer, diamondback moth, green cloverworm, imported cabbageworm, melonworm, omnivorous leafroller, pickleworm, rindworm complex, saltmarsh caterpillar, soybean looper, tobacco budworm, tomato fruitworm, tomato homworrri, tomato pinworm, velvetbean caterpillar, and yellow-striped annyworm.

Likewise, pasture and hay crops such as alfalfa, pasture grasses and silage are often attacked by pests, such as annyworm, beef annyworm, alfalfa caterpillar, European skipper, a variety of loopers and webworms, as well as yellowstriped armyworms.

Fruit and vine crops are often susceptible to attack and defoliation by achema sphinx moth, amorbia, armyworm, citrus cutworm, banana skipper, blackheaded fireworm, blueberry leafroller, cankerworm, cherry fruitworm, citrus cutworm, cranberry girdler, eastern tent caterpillar, fall webworm, fall webworm, filbert leafroller, filbert webworm, fruit tree leafroller, grape berry moth, grape leaffolder, grapeleaf skeletonizer, green fruitworm, gummosos-batrachedra commosae, gypsy moth, hickory shuckworm, hornworms, loopers, navel orangeworm, obliquebanded leafroller, orrinivorous leafroller, omnivorous looper, orange tortrix, orangedog, oriental fruit moth, pandemis leafroller, peach twig borer, pecan nut casebearer, redbanded leafroller, redhumped caterpillar, roughskinned cutworm, saltmarsh caterpillar, spanworm, tent caterpillar, thecla-thecla basillides, tobacco budworm, tortrix moth, tufted apple budmoth, variegated leafroller, walnut caterpillar, western tent caterpillar, and yellowstriped annyworm.

Field crops such as canola/rape seed, evening primrose, meadow foam, corn (field, sweet, popcorn), cotton, hops, jojoba, peanuts, rice, safflower, small grains (barley, oats, rye, wheat, etc.), sorghum, soybeans, sunflowers, and tobacco are often targets for infestation by insects, including: armyworm, asian and other corn borers, banded sunflower moth, beet annyworm, bollworm, cabbage looper, corn rootworm (including southern and western varieties), cotton leaf perforator, diamondback moth, european corn borer, green cloverworm, headmoth, headworm, imported cabbagewonn, loopers (including Anacamptodes spp.), obliquebanded leafroller, omnivorous leaftier, podworin, podworm, saltmarsh caterpillar, southwestern corn borer, soybean looper, spotted cutworm, sunflower moth, tobacco budworm, tobacco hornworm, velvetbean caterpillar, Bedding plants, flowers, ornamentals, vegetables and container stock are frequently fed upon by a host of insect pests such as armyworm, azalea moth, beet armyworm, diamondback moth, ello moth (hornworm), Florida fern caterpillar, lo moth, loopers, oleander moth, omnivorous leafroller, omnivorous looper, and tobacco Forests, fruit, ornamental, and nut-bearing trees, as well as shrubs and other nursery stock are often susceptible to attack from diverse insects such as bagworm, blackheaded budworm, browntail moth, California oakworm, douglas fir tussock moth, elm spanworm, fall webworm, fruit tree leafroller, greenstriped mapleworm, gypsy moth, Jack pine budworm, mimosa webworm, pine butterfly, redhumped caterpillar, saddleback caterpillar, saddle prominent caterpillar, spring and fall cankerworm, spruce budworm, tent caterpillar, tortrix, and western tussock moth.

Likewise, turf grasses are often attacked by pests such as armyworm, sod webworm, and tropical sod webworm.

It is also envisioned that the methods described herein are also applicable to pest control, wherein the pests are not insects but rather, e.g., nematodes, slugs or snails.

In one embodiment, the methods described herein are also applicable to insects that are parasites. Examples of some insect parasites are Braconid Wasps, family Braconidae; Ichneumonid Wasps, family Ichneumonidae; Chalcid Wasps, family Chalcidae; Tachinid Flies, family Tachonidae.

In certain other embodiments, the methods described herein may also be applicable to insects that are disease vectors. Vectors are organisms that can introduce a pathogen such as a bacterium or virus into a host organism to cause an infection or disease. Exemplary disease vector include, but are not limited to, mosquitoes, Ticks, Siphonaptera (fleas), Diptera (flies), Phthiraptera (lice) and Hemiptera (true bugs).

Once a compound suitable for insecticidal use is identified, the active ingredient, or formulations comprising them, may be applied directly to the target insects (i.e., larvae, pupae and/or adults), or to the locus of the insects. In one embodiment, the active ingredient or a formulation and/or composition containing the active ingredient is applied directly to the adult insect. In one embodiment, the active agent is applied directly to the larvae and/or pupae of the target insect. In another embodiment, the active ingredient is applied to the locus of the insects.

Because compounds incorporating hydrophobic moieties will penetrate the insect cuticle, active agents can be conjugated with hydrophobic moieties. Hydrophobic moieties include, but are not limited to, lipids and sterols.

In one embodiment, the active ingredient or a formulation including the active ingredient may be applied as a spray. For example, the active ingredient may be applied as an agricultural spray in aerial crop dusting, an environmental spray to control biting insects, or as a topical spray for localized control of biting insects. The active ingredient may be formulated for the purpose for spray application such as an aerosol formulation. Spray application may be accomplished with a spray pump. The active ingredient may be also encapsulated within materials such as starch, flour and gluten in granular formulations.

In certain embodiments, the active ingredient or a formulation including the active ingredient may be applied in conjunction with other insecticides and/or pesticides such as organo-phosphates, synthetic pyrethroids, carbamates, chlorinated hydrocarbons, when used in agricultural and/or environmental insect control.

The active ingredient may be administered in an amount effective to induce the desired response as determined by routine testing. The actual effective amount will of course vary with the specific active ingredient, the target insect and its stage of development, the application technique, the desired effect, and the duration of the effect, and may be readily determined by the practitioner skilled in the art. “An effective amount of active ingredient” refers to the amount of active ingredient that modulates (activates or inhibits) an insect TRPV channel, e.g., modulates feeding behavior of an insect to achieve the desired insect control.

Methods of formulation are well known to one skilled in the art and are also found in Knowles, D A (1998) Chemistry and technology of agricultural formulations. Kluwer Academic, London, which is hereby incorporated by reference in its entirety. One skilled in the art will, of course, recognize that the formulation and mode of application may affect the activity of the active ingredient in a given application. Thus, for agricultural and/or horticultural use the TRPV inhibitors and/or agonists may be formulated as a granular of relatively large particle size (for example, 8/16 or 4/8 US Mesh), as water-soluble or water-dispersible granules, as powdery dusts, as wettable powders, as emulsifiable concentrates, as aqueous emulsions, as solutions, as suspension concentrate, as capsule suspensions, as soluble (liquid) concentrates, as soluble powders, or as any of other known types of agriculturally-useful formulations, depending on the desired mode of application. It is to be understood that the amounts specified in this specification are intended to be approximate only, as if the word “about” were placed in front of the amounts specified.

These formulations may be applied either as water-diluted sprays, or dusts, or granules in the areas in which insect control is desired. The formulations may contain as little as 0.1%, 0.2% or 0.5% to as much as 95% or more by weight of active ingredient, e.g. insect TRPV inhibitor.

“Dusts” are free flowing admixtures of the active ingredient with finely divided solids such as talc, natural clays, kieselguhr, flours such as walnut shell and cottonseed flours, and other organic and inorganic solids which act as dispersants and carriers for the toxicant; these finely divided solids have an average particle size of less than about 50 microns. A typical dust formulation useful herein is one containing 90 parts, 80 parts, 70 parts, 60 parts, 50 parts, 40 parts, 30 parts, 20 parts, preferably 10 parts, or less of the active ingredient, e.g., insect TRPV inhibitor or insect TRPV agonist. In one embodiment, the dust formulation may include 1 part or less of the active ingredient and 99 parts or more of talc.

Wettable powders, useful as formulations, are in the form of finely divided particles that disperse readily in water or other dispersant. The wettable powder is ultimately applied to the locus where insect control is needed either as a dry dust or as an emulsion in water or other liquid. Typical carriers for wettable powders include Fuller's earth, kaolin clays, silicas, and other highly absorbent, readily wet inorganic diluents. Wettable powders typically are prepared to contain about 5-80% of active ingredient, depending on the absorbency of the carrier, and usually also contain a small amount of a wetting, dispersing or emulsifying agent to facilitate dispersion. For example, a useful wettable powder formulation contains 80.0 parts of the active ingredient, 17.9 parts of Palmetto clay, and 1.0 part of sodium lignosulfonate and 0.3 part of sulfonated aliphatic polyester as wetting agents. Additional wetting agent and/or oil may be added to a tank mix for to facilitate dispersion on the foliage of the plant.

Other useful formulations are emulsifiable concentrates (ECs) which are homogeneous liquid compositions dispersible in water or other dispersant, and may consist entirely of the active ingredient, and a liquid or solid emulsifying agent, or may also contain a liquid carrier, such as xylene, heavy aromatic naphthas, isophorone, or other non-volatile organic solvents. For insecticidal application these concentrates are dispersed in water or other liquid carrier and normally applied as a spray to the area to be treated. The percentage by weight of the essential active ingredient may vary according to the manner in which the composition is to be applied, but in general comprises 0.5 to 95% of active ingredient by weight of the insecticidal composition.

Flowable formulations are similar to ECs, except that the active ingredient is suspended in a liquid carrier, generally water. Flowables, like ECs, may include a small amount of a surfactant, and will typically contain active ingredients in the range of 0.5 to 95%, frequently from 10 to 50%, by weight of the composition. For insecticidal application, flowables may be diluted in water or other liquid vehicle, and are typically applied as a spray.

Typical wetting, dispersing or emulsifying agents used in agricultural and/or horticultural formulations may include, but are not limited to, the alkyl and alkylaryl sulfonates and sulfates and their sodium salts; alkylaryl polyether alcohols; sulfated higher alcohols; polyethylene oxides; sulfonated animal and vegetable oils; sulfonated petroleum oils; fatty acid esters of polyhydric alcohols and the ethylene oxide addition products of such esters; and the addition product of long-chain mercaptans and ethylene oxide. Many other types of useful surface-active agents are available in commerce. Surface-active agents, when used, typically include 1 to 15% by weight of the composition.

Other useful formulations include suspensions of the active ingredient in a relatively non-volatile solvent such as water, corn oil, kerosene, propylene glycol, or other suitable solvents.

In certain embodiments, formulations for insecticidal applications may include simple solutions of the active ingredient in a solvent, in which it is completely soluble at the desired concentration, such as acetone, alkylated naphthalenes, xylene, or other organic solvents.

Granular formulations, wherein the active ingredient is carried on relative coarse particles, are of particular utility for aerial distribution or for penetration of cover crop canopy. Pressurized sprays, typically aerosols wherein the active ingredient is dispersed in finely divided form as a result of vaporization of a low-boiling dispersant solvent carrier may also be used. Water-soluble or water-dispersible granules are free flowing, non-dusty, and readily water-soluble or water-miscible. In use by the farmer on the field, the granular formulations, emulsifiable concentrates, flowable concentrates, aqueous emulsions, solutions, etc., may be diluted with water to give a concentration of active ingredient in the range of say 0.1% or 0.2% to 1.5% or 2%.

By far the most frequently used are water-miscible formulations for mixing with water then applying as sprays. Water miscible, older formulations include: emulsifiable concentrate, wettable powder, soluble (liquid) concentrate, and soluble powder. Newer, non-powdery formulations with reduced or no hazardous solvents and improved stability include: suspension concentrate, capsule suspensions, and water dispersible granules. Such formulations are preferably solutions and suspension, e.g., aqueous suspension and solutions, ethanolic suspension and solutions, aqueous/ethanolic suspension and solutions, saline solutions, and colloidal suspensions.

Alternatively, a sprayable wax emulsion formulation may be used. The formulation contains the active ingredient, in an amount from about 0.01% to 75% by weight. The aqueous wax emulsions are broadly described in U.S. Pat. No. 6,001,346, which is hereby incorporated by reference in its entirety. The TRPV inhibitors of the methods described herein can have a viscosity appropriate for use in aerial or backpack spray applications.

The biodegradable wax carrier comprises at least about 10% by weight of the formulation. The biodegradable wax carrier is selected from the group consisting of paraffin, beeswax, vegetable based waxes such as soywax (soybean based), and hydrocarbon based waxes such as Gulf Wax Household Paraffin Wax; paraffin wax, avg. m.p. 53C (hexacosane), high molecular weight hydrocarbons), carnauba wax, lanolin, shellac wax, bayberry wax, sugar cane wax, microcrystalline, ozocerite, ceresin, montan, candelilla wax, and combinations thereof.

Formulations may also contain an emulsifier in an amount from about 1% to about 10% by weight. Suitable emulsifiers include lecithin and modified lecithins, mono- and diglycerides, sorbitan monopalmitate, sorbitan monooleate, sorbitan monolaurate, polyoxyethylene-sorbitan monooleate, fatty acids, lipids, etc. The emulsifiers provide or improve emulsification properties of the composition. The emulsifier can be selected from many products which are well known in the art, including, but not limited to, sorbitan monolaurate (anhydrosorbitol stearate, molecular formula C₂₄H₄₆O₆), ARLACEL 60, ARMOTAN MS, CRILL 3, CRILL K3, DREWSORB 60, DURTAN 60, EMSORB 2505, GLYCOMUL S, HODAG SMS, IONET S 60, LIPOSORB S, LIPOSORB S-20, MONTANE 60, MS 33, MS33F, NEWCOL 60, NIKKOL SS 30, NISSAN NONION SP 60, NONION SP 60, NONION SP 60R, RIKEMAL S 250, sorbitan c, sorbitan stearate, SORBON 60, SORGEN 50, SPAN 55, AND SPAN 60; other sorbitan fatty acid ester that may be used include sorbitan monopalmitate, sorbitan monostearate, sorbitan tristearate, sorbitan monooleate, sorbitan sesquioleate, sorbitan trioleate, sorbitan monooleate, sorbitan trioleate.

In certain embodiments, formulations can include a phagostimulant, such as corn oil, molasses, glycerol, or corn syrup, proteinaceous material (protein or hydrolyzed protein), sugars like sucrose, or food-based ingredients such as trimethylamine, putrescine, bacterial or yeast volatiles or metabolites, ammonium acetate, ammonium carbonate or other ammonia-emitting compounds. Acetic acid vapor can be provided by compounds that produce volatilized acetic acid, for example, aqueous acetic acid, glacial acetic acid, glacial (concentrated) acetic acid, or ammonium producing compounds such as but not restricted to ammonium hydroxide, ammonium carbonate, ammonium bicarbonate, ammonium acetate, etc.

In certain embodiments, the active ingredient may be formulated and/or applied with one or more second compounds. For example, various combinations of TRPV inhibitors and TRPV agonists may be used to obtain greater advantage. For example, both a TRPV inhibitor and TRPV agonist may be applied at the same time. As such in one embodiment, a formulation described herein may include both, a TRPV inhibitor and a TRPV agonist. In one embodiment, two or more active agents may be formulated together. In alternative embodiment, two or more active agents formulated together are all either TRPV inhibitors or are all TRPV agonists. Such combinations may provide certain advantages, such as, without limitation, exhibiting synergistic effects for greater control of insects or non-insect pests, reducing rates of application thereby minimizing any impact to the environment and to worker safety, controlling a broader spectrum of insects and non-insect pests, and improving tolerance by non-pest species, such as mammals and fish. Other second compounds include, without limitation, insecticides, pesticides, plant growth regulators, fertilizers, soil conditioners, or other agricultural and horticultural chemicals. The formulation may include such second compounds in an amount from about 0.002% to about 25% by weight of the composition.

In certain embodiments, the formulations of the present invention may contain visual attractants, e.g. food coloring.

A variety of additives may also be incorporated into the formulation. These additives typically change and/or enhance the physical characteristics of the carrier material and are, therefore, suitable for designing compositions having specific requirements as to the release rate and amount of the active ingredient, protection of the wax composition from weather conditions, etc. These additives are, among others, plasticizers, volatility suppressants, antioxidants, lipids, various ultraviolet blockers and absorbers, or antimicrobials, typically added in amounts from about 0.001% to about 10%, more typically between 1-6%, by weight.

Plasticizers, such as glycerin or soy oil affect physical properties of the composition and may extend its resistance to environmental destruction.

Antioxidants, such as vitamin E, BHA (butylated hydroxyanisole), BHT (butylated hydroxytoluene), and other antioxidants which protect the bioactive agent from degradation, may be added in amounts from about 0.1% to about 3%, by weight.

Ultraviolet blockers, such as beta-carotene, lignin or p-aminobenzoic acid protect the bioactive agents from light degradation may be added in amounts from about 1% to about 3%, by weight.

Antimicrobials, such as potassium sorbate, nitrates, nitrites, and propylene oxide, protect the bioactive agents from microbial destruction may be added in amounts from 0.1% to about 2% by weight.

Adjuvants can also be added to the formulation. An “adjuvant” is broadly defined as any substance added to the spray tank, separate from the insecticide formulation that will improve the performance of the insecticide. These may include, but are not limited to: wetter-spreaders, stickers, penetrants, compatibility agents, buffers, and so on.

Other compounds and materials may also be added provided they do not substantially interfere with the activity of active ingredient. Whether or not an additive substantially interferes with the active ingredient's activity can be determined by standard test formats, involving direct comparisons of efficacy of the composition of the active ingredient without an added compound and the composition of the active ingredient with an added compound.

It was discovered that an insect will stop eating after ingesting a TRP and/or TRPV channel activating compound. For example ingesting a TRPV ion gated channel agonist can cause an insect to stop eating. Thus, in one embodiment, the compounds may be formulated with a food source for insects, e.g., formulated with compounds in insect diet. In another embodiment, the compounds may be formulated with sucrose. Without wishing to be bound by theory, the insect will feed on such mixtures and stop eating.

In certain embodiments, the active agent may be applied to feeding locus of insects. This inhibits insect feeding leading to starvation of insects. In one embodiment, the active agent is applied as a spray to locus of insects, e.g., feeding locus. The active agent may be applied to the insect and/or pest (mollusk, nematode and/or arthropod) feeding source, used in bait or traps, and or directly applied to the pest itself.

In one embodiment, the active agent may be applied to insect traps. For example, the trap may be coated with the active agent or trap may be loaded with insect food comprising an active agent.

The nucleic acid sequences of Nanchung and Inactive subunits of the TRPV channel as well as the corresponding amino acid sequences, their origins and accession numbers (if available) are summarized in Table 1 below. Upon co-expression of the Nanchung and Inactive proteins in a cell, a functional TRPV channel may be formed.

TABLE 1

| SEQ | | | |
|---|---|---|---|
| ID | | | |
| NO. | DESCRIPTION | ORGANISM | ACCESSION NO. |
| 1 | Nanchung DNA | Drosophila melanogaster | NM_140439.3 |
| 2 | Nanchung DNA | Drosophila melanogaster | NM_001274904.1 |
| 3 | Nanchung DNA | Musca domestica | XM_005180432 |
| 4 | Nanchung DNA | Ceratitis capitata | XM_004537685.1 |
| 5 | Nanchung DNA | Anopheles gambiae | XM_320300.4 |
| 6 | Nanchung DNA | Aedes aegypti | XM_001652374.1 |
| 7 | Nanchung DNA | Culex quinquefasciatus | XM_001847084 |
| 8 | Nanchung DNA | Tribolium castaneum | XM_962803.1 |
| 9 | Nanchung DNA | Bombyx mori | XM_004923013.1 |
| 10 | Nanchung DNA | Anopheles darlingi | gi\|312376269 |
| 11 | Nanchung DNA | Acyrthosiphon pisum | XM_001947872.2 |
| 12 | Nanchung DNA | Dendroctonus ponderosae | gi\|459669722 |
| 13 | Nanchung DNA | Harpegnathos saltator | gi\|307201159 |
| 14 | Nanchung DNA | Nasonia vitripennis | XM_001606052.2 |
| 15 | Nanchung DNA | Megachile rotundata | XM_003706124.1 |
| 16 | Nanchung DNA | Apis mellifera | XM_625167.3 |
| 17 | Nanchung DNA | Apis florea | XM_003689958.1 |
| 18 | Nanchung DNA | Pediculus humanus | XM_002427918.1 |
| | | corporis | |
| 19 | Nanchung DNA | Danaus plexippus | gi\|357621160 |
| 20 | Nanchung DNA | Solenopsis invicta | gi\|322788678 |
| 21 | Nanchung DNA | Acromyrmex echinatior | gi\|332030918 |
| 22 | Nanchung DNA | Camponotus floridanus | gi\|307175924 |
| 23 | Nanchung DNA | Myzus persicae | In-house |
| 24 | Nanchung DNA | Bemisia tabaci | In-house |
| 25 | Nanchung DNA | Euschistus heros | In-house |
| 26 | Nanchung DNA | Nilaparvata lugens | In-house |
| 27 | Nanchung DNA | Schistocerca americana | In-house |
| 28 | Inactive DNA | Drosophila melanogaster | NM_132125.1 |
| 29 | Inactive DNA | Musca domestica | XM_005180960.1 |
| 30 | Inactive DNA | Ceratitis capitata | XM_004529418.1 |
| 31 | Inactive DNA | Aedes aegypti | XM_001659838.1 |
| 32 | Inactive DNA | Culex quinquefasciatus | XM_001864290.1 |
| 33 | Inactive DNA | Anopheles gambiae | XM_310685.5 |
| 34 | Inactive DNA | Tribolium castaneum | TC012368 |
| 35 | Inactive DNA | Megachile rotundata | XM_003704618.1 |
| 36 | Inactive DNA | Apis mellifera | XM_001121881.2 |
| 37 | Inactive DNA | Apis florea | XM_003690679.1 |
| 38 | Inactive DNA | Pediculus humanus | XM_002432337.1 |
| | | corporis | |
| 39 | Inactive DNA | Acromyrmex echinatior | gi\|332020355 |
| 40 | Inactive DNA | Harpegnathos saltator | gi\|307206889 |
| 41 | Inactive DNA | Bombus impatiens | XM_003485651.1 |
| 42 | Inactive DNA | Bombus terrestris | XM_003396143.1 |
| 43 | Inactive DNA | Dendroctonus ponderosae | gi\|478256802 |
| 44 | Inactive DNA | Nasonia vitripennis | XM_001602538.2 |
| 45 | Inactive DNA | Camponotus floridanus | gi\|307168683 |
| 46 | Inactive DNA | Danaus plexippus | gi\|357618515 |
| 47 | Inactive DNA | Bombyx mori | XM_004925264.1 |
| 48 | Inactive DNA | Acyrthosiphon pisum | XM_001950061.2 |
| 49 | Inactive DNA | Solenopsis invicta | GL767538.1 |
| 50 | Inactive DNA | Schistocerca americana, | In-house |
| | | DNA partial | |
| 51 | Inactive DNA | Myzus persicae | In-house |
| 52 | Inactive DNA | Bemisia tabaci | In-house |
| 53 | Inactive DNA | Euschistus heros | In-house |
| 54 | Nanchung PROT | Drosophila melanogaster | NP_648696.2 |
| 55 | Nanchung PROT | Drosophila melanogaster | NP_001261833.1 |
| 56 | Nanchung PROT | Musca domestica | XP_005180489.1 |
| 57 | Nanchung PROT | Ceratitis capitata | XP_004537742.1 |
| 58 | Nanchung PROT | Anopheles gambiae | XP_320300.4 |
| 59 | Nanchung PROT | Aedes aegypti | XP_001652424.1 |
| 60 | Nanchung PROT | Culex quinquefasciatus | XP_001847136.1 |
| 61 | Nanchung PROT | Tribolium castaneum | XP_967896.1 |
| 62 | Nanchung PROT | Bombyx mori | XP_004923070.1 |
| 63 | Nanchung PROT | Anopheles darlingi | EFR23411.1 |
| 64 | Nanchung PROT | Acyrthosiphon pisum | XP_001947907.2 |
| 65 | Nanchung PROT | Dendroctonus ponderosae | ERL84850.1 |
| 66 | Nanchung PROT | Harpegnathos saltator | EFN81068.1 |
| 67 | Nanchung PROT | Nasonia vitripennis | XP_001606102.2 |
| 68 | Nanchung PROT | Megachile rotundata | XP_003706172.1 |
| 69 | Nanchung PROT | Apis mellifera | XP_625170.3 |
| 70 | Nanchung PROT | Apis florea | XP_003690006.1 |
| 71 | Nanchung PROT | Pediculus humanus | XP_002427963.1 |
| | | corporis | |
| 72 | Nanchung PROT | Danaus plexippus | EHJ73092.1 |
| 73 | Nanchung PROT | Solenopsis invicta | EFZ14282.1 |
| 74 | Nanchung PROT | Acromyrmex echinatior | EGI70573.1 |
| 75 | Nanchung PROT | Camponotus floridanus | EFN65752.1 |
| 76 | Nanchung PROT | Myzus persicae | In-house |
| 77 | Nanchung PROT | Bemisia tabaci | In-house |
| 78 | Nanchung PROT | Euschistus heros | In-house |
| 79 | Nanchung PROT | Nilaparvata lugens | In-house |
| 80 | Nanchung PROT | Schistocerca americana | In-house |
| 81 | Inactive PROT | Drosophila melanogaster | NP_572353.1 |
| 82 | Inactive PROT | Musca domestica | XP_005181017.1 |
| 83 | Inactive PROT | Ceratitis capitata | XP_004529475.1 |
| 84 | Inactive PROT | Aedes aegypti | XP_001659888.1 |
| 85 | Inactive PROT | Culex quinquefasciatus | XP_001864325.1 |
| 86 | Inactive PROT | Anopheles gambiae | XP_310685.5 |
| 87 | Inactive PROT | Tribolium castaneum | EFA10736.1 |
| 88 | Inactive PROT | Megachile rotundata | XP_003704666.1 |
| 89 | Inactive PROT | Apis mellifera | XP_001121881.1 |
| 90 | Inactive PROT | Apis florea | XP_003690727.1 |
| 91 | Inactive PROT | Pediculus humanus | XP_002432382.1 |
| | | corporis | |
| 92 | Inactive PROT | Acromyrmex echinatior | EGI60788.1 |
| 93 | Inactive PROT | Harpegnathos saltator | EFN84766.1 |
| 94 | Inactive PROT | Bombus impatiens | XP_003485699.1 |
| 95 | Inactive PROT | Bombus terrestris | XP_003396191.1 |
| 96 | Inactive PROT | Dendroctonus ponderosae | ERL85467.1 |
| 97 | Inactive PROT | Nasonia vitripennis | XP_001602588.2 |
| 98 | Inactive PROT | Camponotus floridanus | EFN61724.1 |
| 99 | Inactive PROT | Danaus plexippus | EHJ71463.1 |
| 100 | Inactive PROT | Bombyx mori | XP_004925321.1 |
| 101 | Inactive PROT | Acyrthosiphon pisum | XP_001950096.1 |
| 102 | Inactive PROT | Solenopsis invicta | EFZ13594.1 |
| 103 | Inactive PROT | Schistocerca americana, | In-house |
| | | DNA partial | |
| 104 | Inactive PROT | Myzus persicae | In-house |
| 105 | Inactive PROT | Bemisia tabaci | In-house |
| 106 | Inactive PROT | Euschistus heros | In-house |

Adenovirions, which include the adenovirus expression vectors of the present invention, can be produced using the following methodology. Adenovirons may be used to generate cells used to express the proteins of interest, for example, Nanchung, Inactive, TRP and/or TRPV proteins.

The methods of creating an adenovirus generally involves the steps of introducing the vector containing the gene of interest (e.g., Nan or lay) into a producer cell. The vector containing the gene of interest may be introduced into the producer cell using standard transfection techniques known to one of skill in the art (Zoltukhin et al., Gene Therapy, 6:973-985, 1999). The adenovirus produced by producer cells (packaging cells) are used to transduce a cell of interest. The cell of interest produces the desired proteins.

Preferably, the adenovirions are added to the cells at the appropriate multiplicity of infection according to standard transduction methods appropriate for the particular target cells. Titers of adenovirions to administer can vary, depending upon the target cell type and the particular viral vector, and may be determined by those of skill in the art without undue experimentation.

The cell line may be an insect cell line, such as Sf9 (ATCC# CRL-1711) or Schneider 2 (S2) cells (Life Technologies, #R690-07) (if needed, to introduce a gene of interest into an insect cell transfection techniques may be used among others), African clawed frog (Xenopus laevis) oocytes (to introduce a gene of interest injection techniques may be used), or a mammalian cell line, such as mouse, hamster, human cell line, (to introduce a gene of interest into a mammalian cell several techniques may be used, including but not limited to transfection, adenoviruses, retroviruses, and/or electroporation, etc. . . . ) or any other cell line that does not normally expresses Nanching and Inactive proteins. One example of a mammalian cell line suitable for use with the vector expression system of the present invention includes Chinese hamster ovary (CHO-K1) cells (ATCC# CCL-61).

In certain embodiments, the cell line co-expresses the Nanchung and Inactive proteins at a ratio of 3:1, 2:1, 1:1, 1:2, and 1:3. Preferably, the cell line co-expresses the Nanchung and Inactive proteins at a ratio of 1:1.

In some embodiments, a cell expressing a recombinant nucleic acid sequence encoding a TRPV channel is a cell that has been transformed with an expression vector comprising a nucleotide sequence encoding an insect TRPV channel such as, but not limited to the TRPV channel proteins discloses herein. Methods for transforming cells that would be known to one of ordinary skill in the art include, but are not limited to, infection using viral vectors, lipofection, electroporation, particle bombardment, and transfection. Detailed procedures for representative methods can be found in Sambrook & Russell, 2001, and references cited therein. Useful expression vectors and methods of introducing such vectors into cells or expression of the encoded polypeptide are also known to one of ordinary skill in the art. For example, a plasmid expression vector can be introduced into a cell by calcium-phosphate mediated transfection, DEAE-Dextran-mediated transfection, lipofection, polybrene- or polylysine-mediated transfection, electroporation, or by conjugation to an antibody, gramicidin S, artificial viral envelopes, or other intracellular carriers. A viral expression vector can be introduced into a cell in an expressible form by infection or transduction, for example, or by encapsulation in a liposome.

When a cell expressing a recombinant nucleic acid sequence encoding an insect TRPV channel gene product has been produced, these cells can then be employed in testing candidate compounds for an ability to modulate ion transport in the cell through the TRP and/or TRPV channel. The exemplary methods for testing ion transport in the cells were described above as well as presented in the Examples sections below. Other applicable methods would be known to those of skill in the art upon consideration of this disclosure.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

Modulation Examples FPR Afidopyropen

### Example I: Afidopyropen and Pymetrozine Elicit Characteristic Hind Leg Extension in Grasshoppers

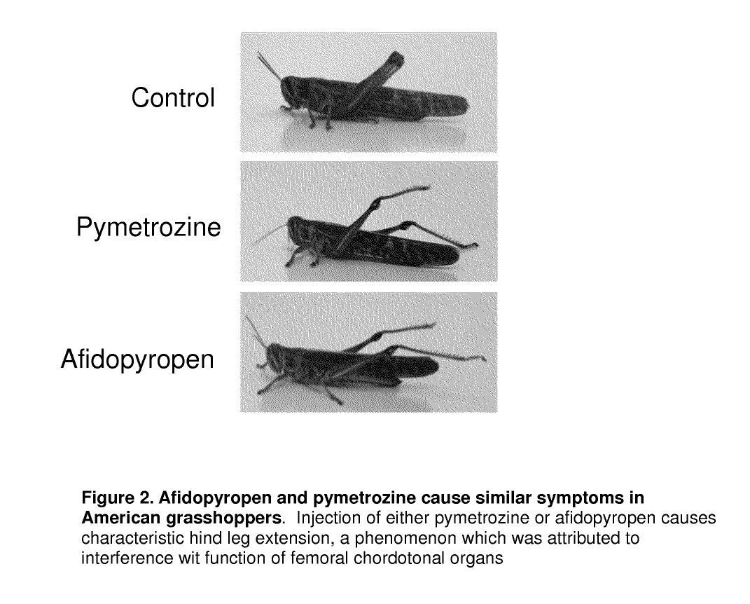

Injection of pymetrozine (PM), was shown to produce characteristic hind leg extension in locusts (Locusta migratoria), a phenomenon which was attributed to silencing mechanosensory neurons in femoral chordotonal organs (Ausborn et al., 2005). In the current invention, the Inventors tested whether afidopyropen targeted insect chordotonal organs in similar fashion, by injecting this compound into American grasshoppers (Schistocerca americana), and found than this injection elicited hind leg extension similarly to pymetrozine (FIG. 2), which suggested that pymetrozine and afidopyropen may act on the same molecular target.

### Example II: Afidopyropen Activates Drosophila TRPV Channels

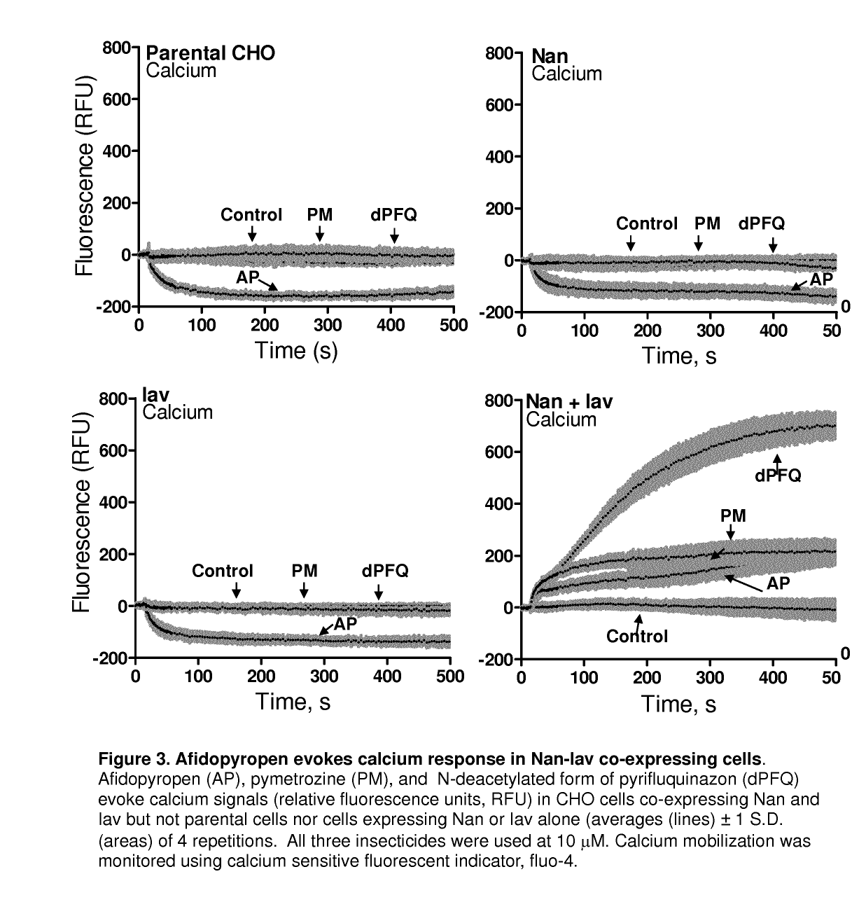

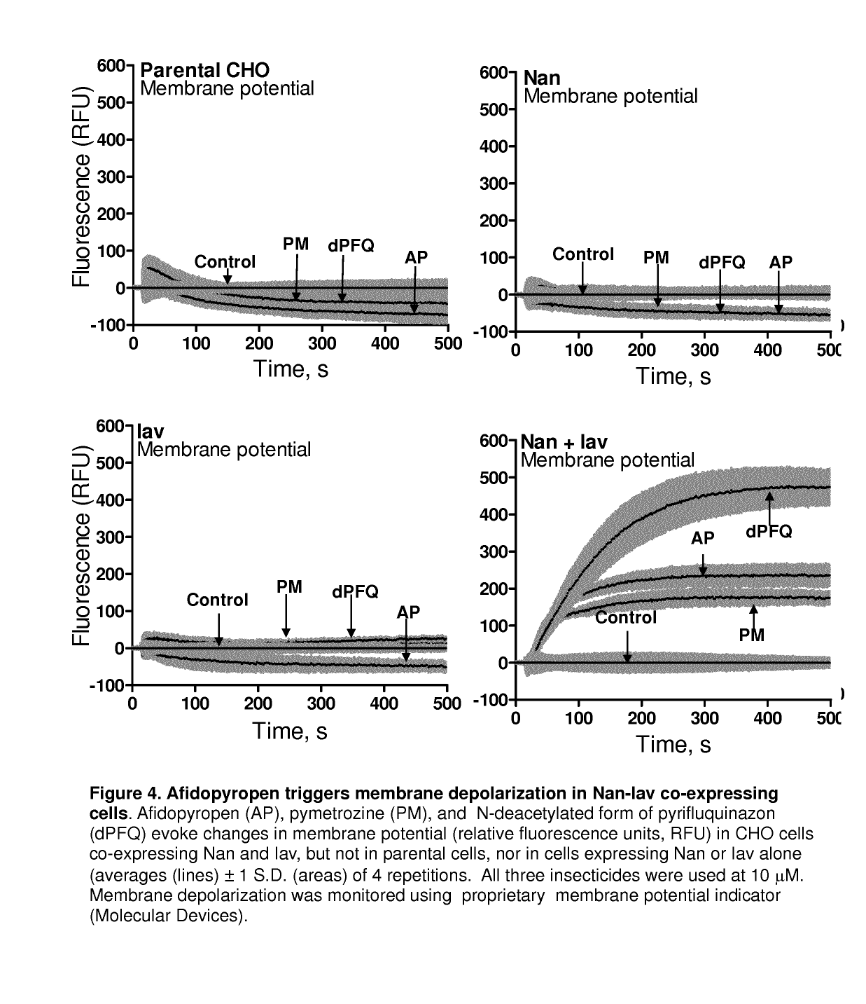

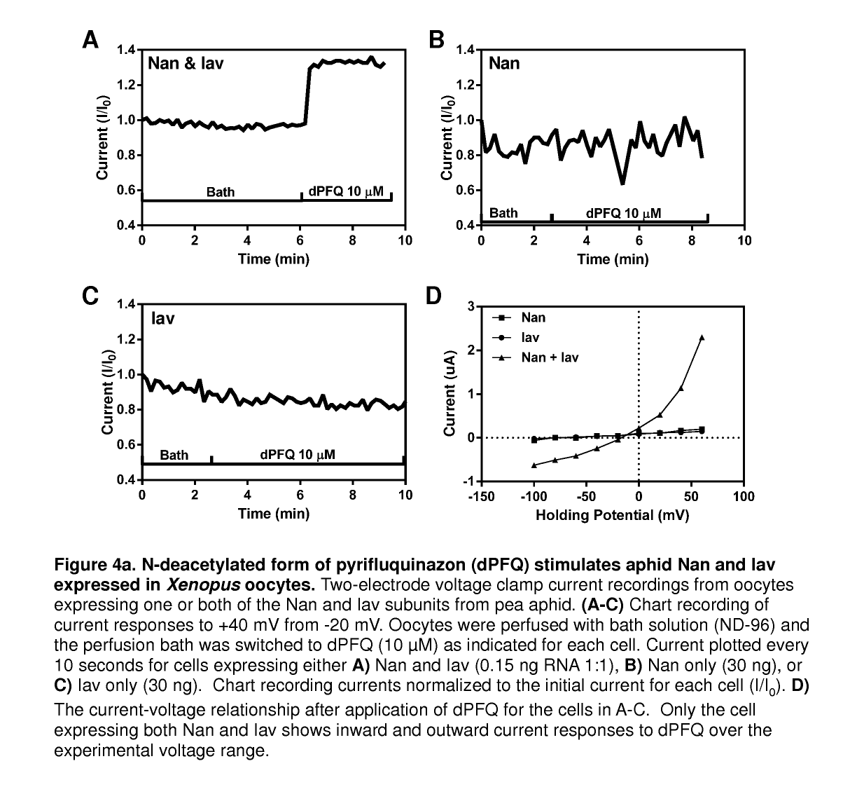

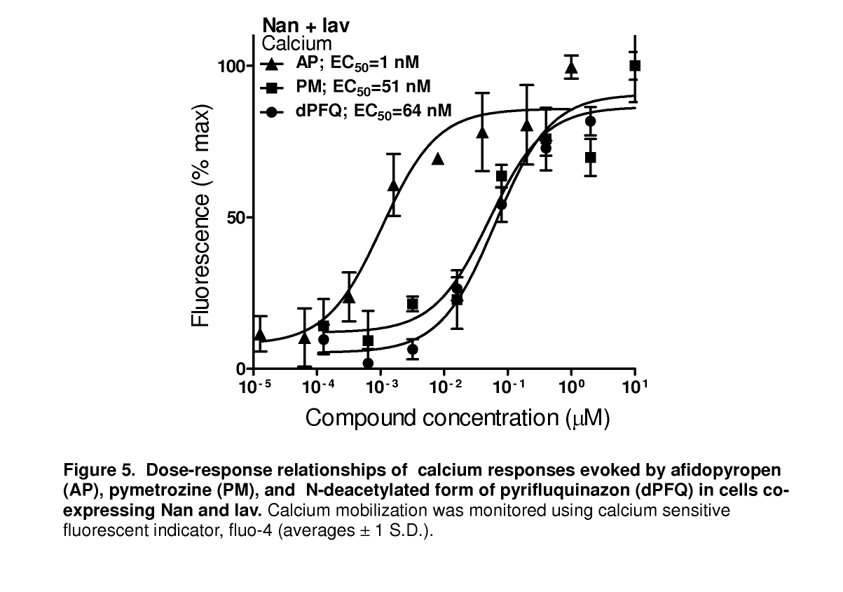

Pymetrozine (PM), pyrifluquinazon and N-deacetylated pyrifluquinazon (dPFQ) elicited calcium mobilization and membrane depolarization in CHO cells (hamster cells) co-infected with adenoviruses expressing insect TRPV channels, Nan and Iav (Nesterov et al., 2013; Nesterov et al., 2015). Using the same approach, the inventors found afidopyropen evoked similar responses in cells co-infected with adenoviruses expressing insect TRPV channels, Nan and Iav. Two different assays were used in these studies: one (FIG. 3) monitored calcium mobilization with calcium probe, fluo-4 (Marshall et al., 2013); whereas another one, (FIG. 4), measured agonist-induced depolarization of plasma with florescent voltage indicator (Zheng et al., 2004). In both assays, afidopyropen, pymetrozine and N-deacetylated pyrifluquinazon triggered calcium mobilization and membrane depolarization responses only in cells co-infected with Nan and Iav viruses, but not in parental cells (e.g. the hamster cells that weren't infected with Nan and/or Iav), nor in cells expressing Nan and/or Iav alone. In the calcium mobilization assay, dose-dependence curves (FIG. 5) yielded the half-maximal effective concentrations (EC₅₀) of 1.0 nM, 64 nM, and 51 nM for afidopyropen, N-deacetylated pyrifluquinazon, and pymetrozine, respectively.

### Example III: N-Deacetylated Pyrifluquinazon Activates TRPV Channels from Pea Aphid

To determine whether activation of TRPV channels from other insect species also requires simultaneous presence of Nan and Iav subunits a two-electrode voltage clamp electrophysiology was used to measure channel opening in Xenopus laevis (frog) oocytes injected with Nan and Iav mRNA from pea aphid, Acyrthosiphon pisum. Current-voltage tests were performed, where currents were measured over a range of holding potentials. N-deacetylated pyrifluquinazon evoked both inward and outward current responses only in oocytes expressing both Nan and Iav (FIG. 4a), but neither Nan no Iav alone.

### Example IV: Afidopyropen does not Activate Mammalian TRPV4 Channel

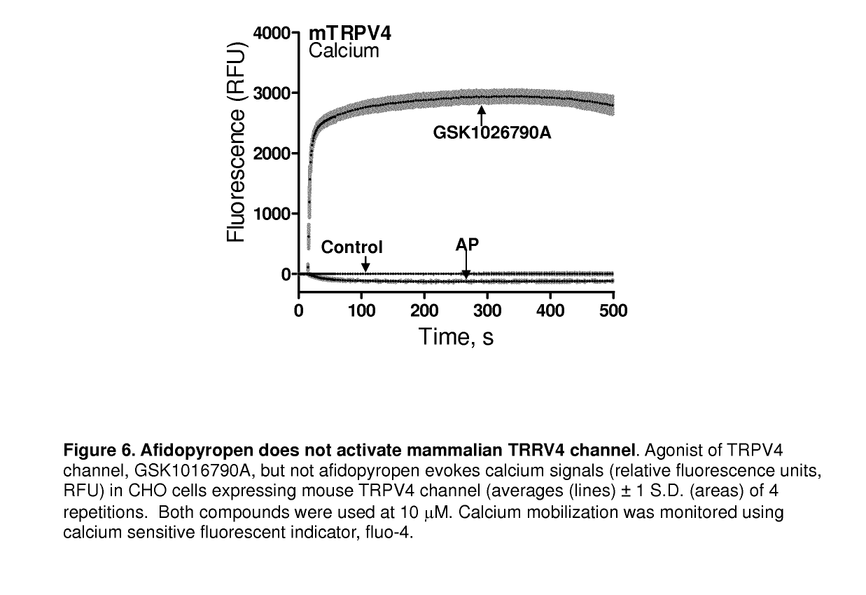

To gain insight into receptor species specificity of afidopyropen effects, CHO cells (hamster cells) were transduced with the adenovirus expressing mouse TRPV channel, mTRPV4. As shown on FIG. 6, the TRPV4 agonist GSK1016790A (Thorneloe et al., 2008), evoked strong calcium response in these cells, whereas afidopyropen failed to activate mTRPV4-expressing cell. GSK1016790A is a known strong agonist of the TRPV4 channel. Accordingly, the inventors found that afidopyropen was a species-specific insecticide, in that it was a modulator of insect TRPV channels but did not modulate the mammalian TRPV4 channel. TRPV4 channel is one of the closest orthologues of the insect TRPV channel. It was chosen to be used based on sequence similarity and the availability of a known agonist.

Direct Binding Examples

### Example V: Afidopyropen Directly Binds Insect TRPV Channel

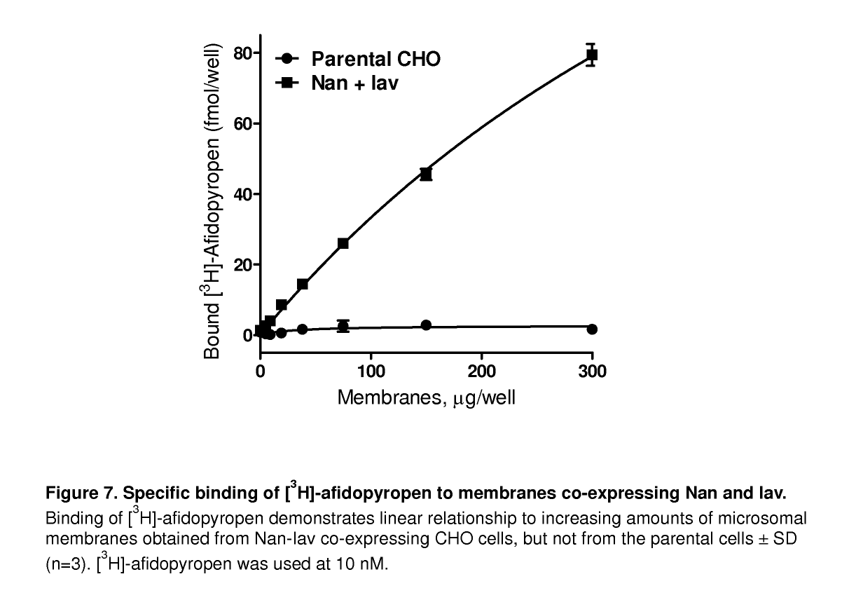

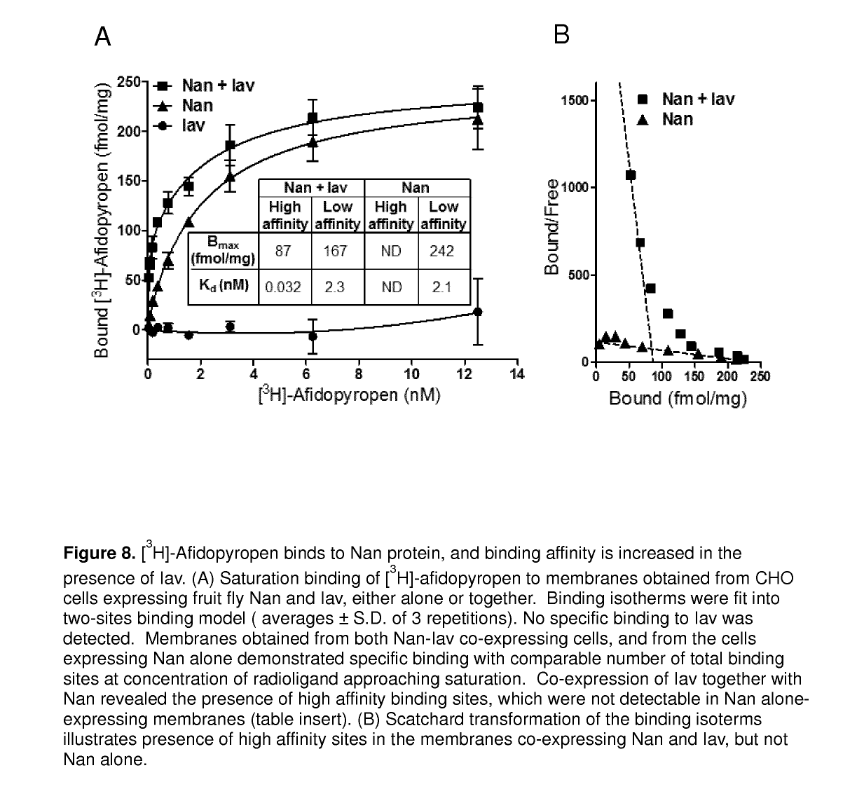

Specific binding of tritium-labeled afidopyropen to membranes prepared from Nan and Iav co-expressing cells exhibited a linear relationship to increasing amounts of membranes, whereas binding to parental cell membranes remained low regardless of the amount of membranes used (FIG. 7). Saturation binding (FIG. 8) to Nan-Iav membranes revealed the presence of 2 binding sites: one of very high affinity (K_{d}=0.032 nM, Bₘₐₓ=87 fmol/mg of membrane proteins), and one of lower affinity (K_{d}=2.3 nM, Bₘₐₓ=167 fmol/mg of membrane proteins). In membranes prepared from Nan-only cells, only lower affinity sites were detected (K_{d}=2.1 nM, Bₘₐₓ=242 fmol/mg of membrane proteins), whereas no specific binding was observed in membranes prepared from Iav-only cells. From this data, the inventors found that the Nan protein forms the major binding interface for afidopyropen, whereas the presence of Iav may act as a facilitator to increase binding affinity.

### Example VI: Pymetrozine and N-Deacetylated Form of Pyrifluquinazon Compete with [³H]-Afidopyropen for Binding

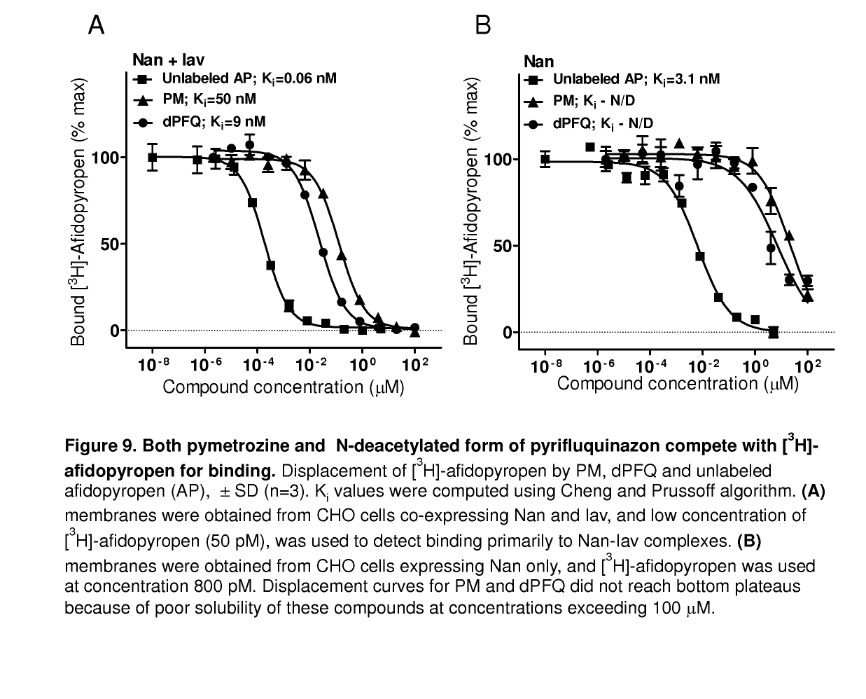

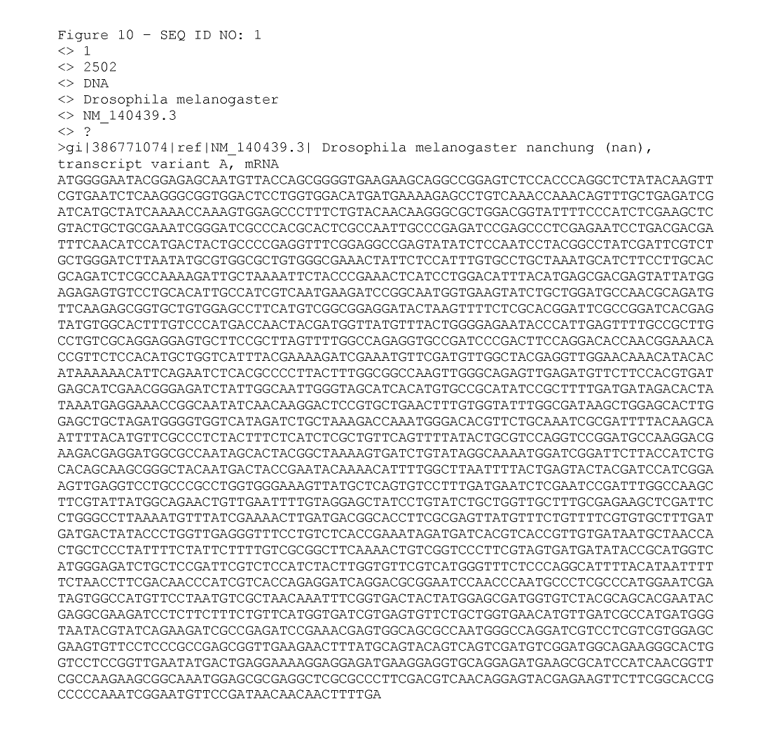

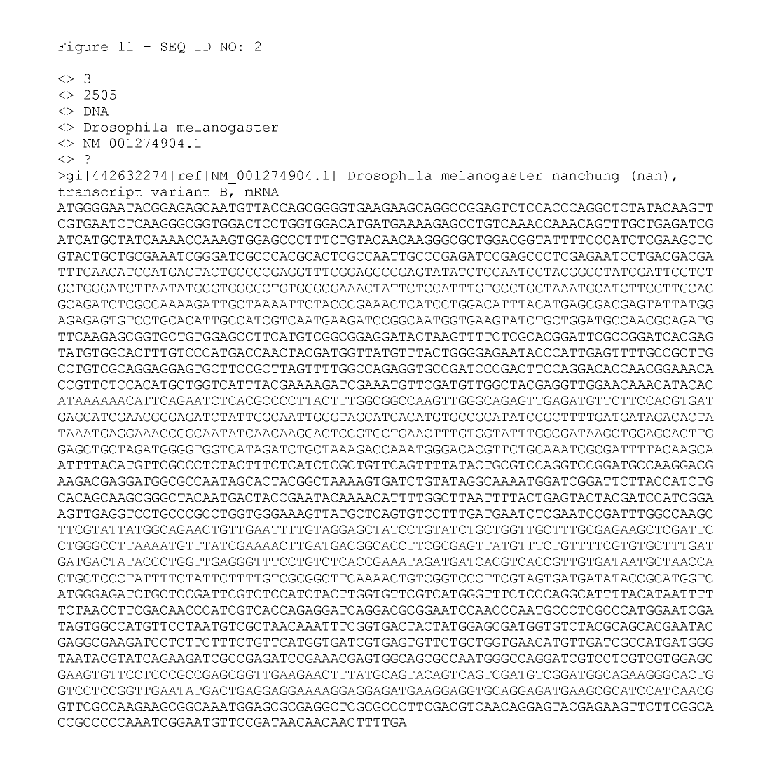

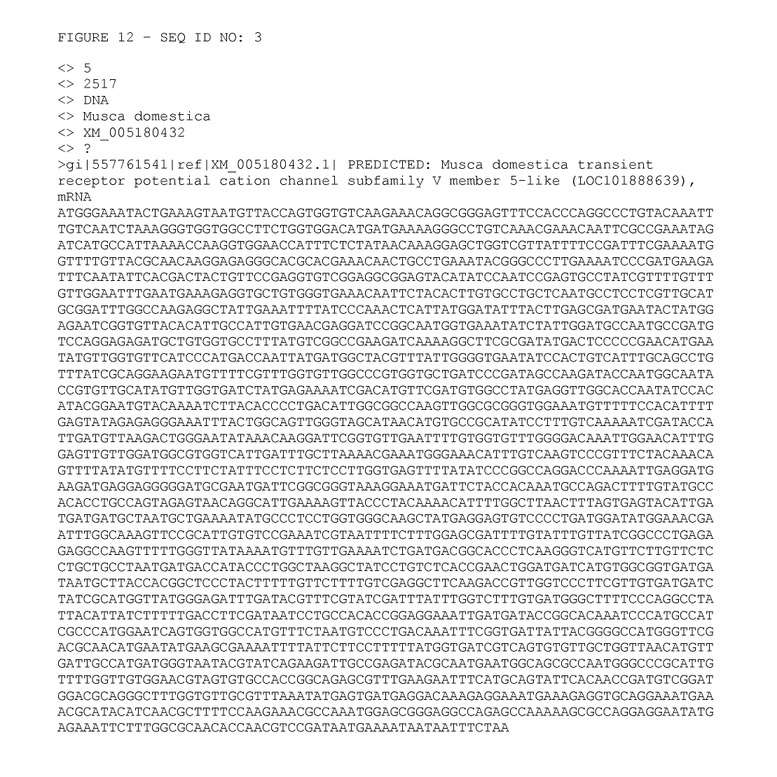

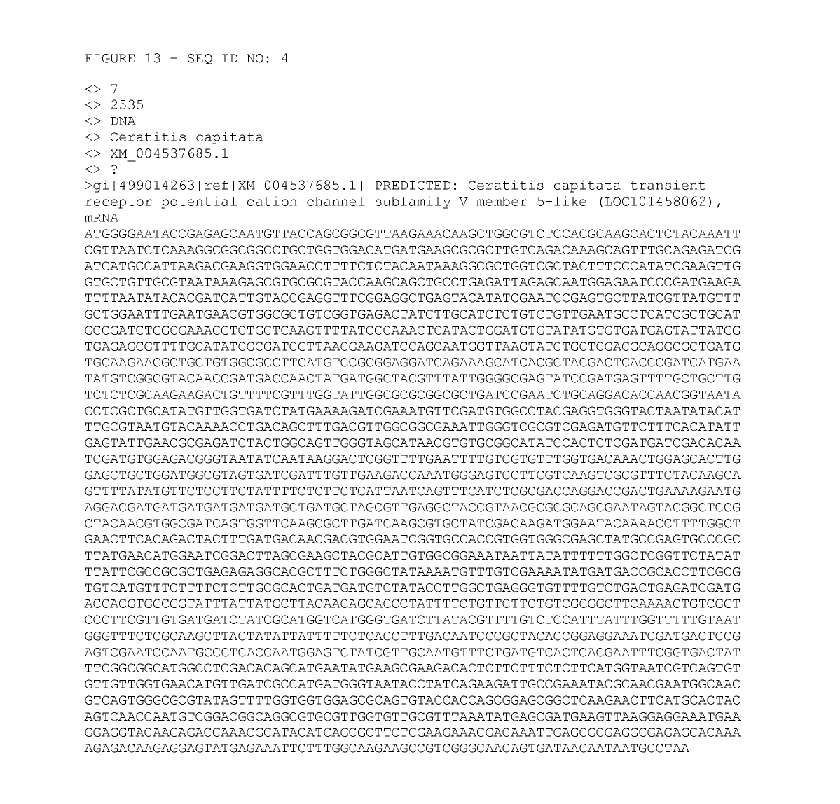

To assess the ability of pymetrozine and N-deacetylated form of pyrifluquinazon to displace [³H]-Afidopyropen from Nan-Iav complexes (FIG. 9A), the experiment was performed at low concentration of [³H]-Afidopyropen (0.05 nM), i.e., under conditions where binding of [³H]-Afidopyropen to Nan-only membranes was negligible (FIG. 8). Both pymetrozine, N-deacetylated pyrifluquinazon and unlabeled Afidopyropen, completely displaced [³H]-Afidopyropen from the high affinity sites with simple kinetics (Hill values≈−1), indicating that all three compounds tested share the same binding site. Noteworthy, for unlabeled Afidopyropen, Kᵢ value of 60 pM measured in displacement assay (FIG. 9A) came close to K_{d} value of 32 pM obtained for [³H]-Afidopyropen in saturating binding experiments (FIG. 8), with less than a two-fold difference, which illustrates close similarity for these two values. N-deacetylated pyrifluquinazon and pymetrozine exhibited significantly lower binding affinities than that of Afidopyropen, with Kᵢ values of 9 nM and 50 nM for N-deacetylated pyrifluquinazon and pymetrozine, respectively, which correlates with lower potencies of these compounds in the functional assay (FIG. 5). Based on this data, the inventors found that afidopyropen directly binds to the same site as pymetrozine and dPFQ, which means these three compounds have the same mode of action.

Displacement of [³H]-Afidopyropen from Nan-only expressing membranes (FIG. 9B) with unlabeled Afidopyropen yielded Kᵢ value of 3.1 nM, which is close to the K_{d} value of 2.1 nM calculated from saturation binding with [³H]-Afidopyropen (FIG. 8). Although pymetrozine and N-deacetylated pyrifluquinazon clearly displaced [³H]-afidopyropen from Nan-only sites, poor solubility of these compounds at high concentrations, prevented competition binding curves from reaching the bottom plateaus, hampering accurate determination of their Kᵢ values. Nevertheless, all three compounds clearly exhibited the same potency ranking (Afidopyropen>N-deacetylated pyrifluquinazon≥pymetrozine) for low affinity Nan-only sites as for high affinity Nan-Iav complexes. It appears, therefore, that co-expression of Iav protein increases binding affinities for all 3 insecticides, despite significant structural differences between Afidopyropen on one hand, and pymetrozine and N-deacetylated pyrifluquinazon on the other hand (FIG. 1). The inventors found that the presence of Inactive protein increased binding affinity for N-deacetylated pyrifluquinazon and pymetrozine, in addition to afidopyropen.

In summary, using the combination of functional and binding assays in the examples above, the inventors demonstrated the following: i) the insect TRPV channel is a direct molecular target of the candidate insecticide, Afidopyropen; ii) Afidopyropen binding site in TRPV protein is shared with two commercial insecticides, pymetrozine and N-deacetylated pyrifluquinazon indicating the same mode of action of all three compounds; iii) binding affinities of all three compounds were measured; iv) Nanchung protein forms the main binding interface for all three compounds, whereas presence of Inactive protein increases binding affinity. (v) new methods were developed which can be used for identifying candidate compounds that may be used as insecticides which act by directly targeting insect TRPV channels (vi) afidopyropen may be used in these methods to assist in the identification of new candidate compounds having same mode of action.

Pymetrozine and GSK10116790A were obtained from Sigma, pyrifluquinazon from ChemService Inc., (West Chester, Pa.), N-deacetylated pyrifluquinazon from Wako Pure Chemical Industries, LTD (Richmond, Va.), and Afidopyropen was synthesized at BASF. Tritium labeled Afidopyropen (19 Ci/mmol) was custom-made by RC TRITEC AG (Teufen, Germany).

Leg Extension Assay

American grasshoppers (Schistocerca americana) were obtained from Dr. Hojun Song (University of Central Florida). Test compounds (10 mM in 100% DMSO), or vehicle (100% DMSO) were applied by injection through the abdominal intersegmental cuticle (3 μl per animal), using Hamilton syringe. The effects of compounds were assessed 40-60 minutes post-injection.

Heterologous Expression of Nan and Iav in CHO-K1 Cells

To produce Nan, Iav and mTRPV4 with fused FLAG tag and AcGFP fluorescent protein at their carboxyl termini, complementary DNAs (cDNAs) encoding Drosophila Nan (NCBI NM_001274904.1) and Iav (NCBI NM_132125.1) were synthesized by Life Technologies (Grand Island, N.Y.), with the addition of sequences encoding FLAG antibody tags at the C-termini. cDNA for mouse TRPV4 (NCBI NM_001274904.1) containing a C-terminal FLAG epitope sequence was purchased from Origene (Rockville, Md.). Insect cDNAs were codon-optimized for expression in mammalian cells. The Nan-FLAG and Iav-FLAG cDNAs were subcloned into the Bgl II and HindIII sites of the modified pAcGFP1-Hyg-N1 vector (Clontech, Kyoto, Japan), which contained a FLAG tag at the C-terminus of the AcGFP moiety (pAcGFP1-Hyg-N1-FLAG). The mTRPV4-FLAG cDNA was PCR amplified using the primers 5′-GGACTTTCCAAAATGTCG-3′ (SEQ ID NO:108) and 5′-CCGGCCGTTTATCACTACAGAATTCGAAGCTTAACCTTATCGTCGTCATCCTTGT A-3′ (SEQ ID NO: 109), subsequently digested with BglII and HindIII, and sub-cloned into the pAcGFP1-Hyg-N1-FLAG vector. These cloning procedures added an AcGFP protein flanked by two FLAG epitope tags to the carboxyl termini of each Nan, Iav and mouse TRPV4.

To produce Nan with fused HA epitopes and mCherry fluorescent protein at its carboxyl terminus, codon-optimized nan cDNA was PCR amplified using the primers 5′-GCTGGTTTAGTGAACCGTCAG-3′ and 5′-CCGCGGTACCGTCGACTGCAGAATTCGAAGCTTAGCGTAATCTGGAACATCGTA TGGGTAGAAGTTGTTGTTGTCGCTGCACTCGGACTTGGG-3′, adding an HA epitope tag to the C-terminus of Nan. The PCR product was digested with XhoI and HindIII, and subcloned into the modified pmCherry-N1 vector (Clontech), which contained an HA tag at the C-terminus of the mCherry moiety. This cloning procedure added mCherry protein flanked by two HA epitope tags to the C-terminus of Nan.

To produce adenoviruses, epitope-tagged Nan and Iav were digested with XhoI and NotI and subcloned to the same sites of the adenovirus shuttle vector pENTCMV1-TetO (Welgen Inc., Worceter, Mass.). Epitope-tagged mTRPV4 was digested with BglII, blunt-ended with Klenow and digested with XbaI. The fragment was subcloned into PmeI and XbaI sites of the pENTCMV1-TetO. pENTCMV1-TetO vectors containing tagged Nan, Iav or mTRPV4 expression constructs were treated with LR Clonase II (Life Technologies) and ligated to a pAdREP plasmid (Welgen), which contains the remaining adenovirus genome. The pENTCMV1-TetO vector contains two Tet repressor binding sites within a modified CMV promoter, which repress transcription of the gene of interest in cells expressing the Tet repressor. The recombination products were transformed into Escherichia coli cells, positive clones were selected, and the cosmid DNA was isolated. The cosmid DNA was digested with Pac I and then transfected into HEK293-TetR cells, which produce the Tet repressor (Postle et al., 1984), preventing expression of TRP channels by the adenovirus packaging cells. Adenoviruses were purified from large-scale cultures and viral titers were calculated as viral particles/mL=Absorption at 260 nm×1.1×10¹⁴.

Heterologous Expression of Nan and Iav in Frog (Xenopus laevis) Oocytes

Nan and Iav coding sequences were RT-PCR amplified from pea aphid (Acyrthosiphon pisum) antennal total RNA using the QIAGEN Onestep RT-PCR Kit (cat#210212) and the primers nan-Ap start 5′-GCCACCATGGGTAACACCGAGAGCAATG-3′, nan-Ap stop 5′-TTATTCATTTGCTTTTAGATTAAATGTCCC-3′, iav-Ap start 5′-GCCACCATGGGAAATTCTTGTGCGAG-3′ and iav-Ap stop 5′-TCAGAAATCGTCGTTGGG-3′, and cloned into the oocyte expression vector pGH19. Linear cDNA templates for transcription were produced by PCR amplification from these constructs using the vector specific primers 5′-GTTGTAAAACGACGGCCAGTGAATTG-3′ and 5′-GACACTATAGAATACTCAAGCTAGCCTCGAGG-3′. These primers were designed to include the T7 promoter and the 3′UTR sequence from the Xenopus globin-XI gene found in pGH19 in the PCR product. The PCR conditions were 1 cycle at 95° C. for 4 minutes, 35 cycles with 95° C. for 30 seconds, 55° C. for 30 seconds and 65° C. for 4 minutes, and 1 cycle at 68° C. for 6 minutes.

In vitro transcription was performed with the T7 polymerase mMessage mMachine kit (Ambion). Xenopus laevis ovaries were obtained from Xenopus 1, Inc. (Dexter, Mich.). The lobes were partially dissected and digested with collagenase type 1 (Alfa Aesar) dissolved in Oocyte Ringers solution (pH 7.5, in mM: NaCl 82.5, KCl 2, MgCl2 1, HEPES 5) until defolliculated. Defolliculated oocytes were washed with and incubated in ND-96⁺ (pH 7.5, in mM: NaCl 96, KCl 2, MgCl2 1, CaCl2 1.8, HEPES 5, Na Pyruvate 2.5 and Gentamycin 50 ug/mL) for two hours before RNA injection. Stage IV and V oocytes were injected with RNA from Nan, Iav or both using a Nanoject automatic injector (3-00-203-X, Drummond Scientific). For one to one expression of each subunit, 0.15 ng of RNA for each subunit was co-injected in 50 nL of RNAse free water (3 ng/uL). For single subunit RNA injections, 30 ng of RNA was injected in 50 nL (600 ng/uL) water to ensure maximal expression of the single subunit. Prior to any measure or comparison steps, oocytes were incubated at 17° C. in ND-96⁺ and the media was changed daily. Further testing steps were made 3 days after RNA injection and/or after sufficient protein production by the oocytes.

Calcium Imaging and Membrane Depolarization Assays

Titers of adenoviruses which allow for equal expression levels of recombinant TRP channels, and optimal cell responses were established previously (Nesterov et al., 2015). Hamster CHO-K1 cells (ATCC® CCL-61™) were infected with adenoviruses expressing AcGFP-Iav, mCherry-Nan, 1:1 mixture of AcGFP-Iav and mCherry-Nan, (4000 viral particles per cell) or AcGFP-mTRPV4 (400 viral particles per cell). The cells were seeded on poly-D-lysine coated 96-well plates (Greiner Bio-One, Frickenhausen, Germany) in 100 μl of media, at a density of 40,000 cells per well. The cells were kept overnight at 37° C., followed by 3 days at 25° C. The medium was changed two days after seeding. Both Ca²⁺ mobilization and changes of membrane potential were measured using a FLIPR-TETRA instrument (Molecular Devices, Sunnyvale, Calif.). Ca²⁺ mobilization was measured using fluo-4AM (Life Technologies). The cells were loaded with 50 μl of Hank's buffered salt solution (HBSS) containing 4 μM fluo-4AM, 5 mM probenecid, 20 mM CaCl₂, and 0.02% pluoronic for 2 hours at 25° C. The dye was then discarded, 50 μl of HBSS were added. The test compounds were dissolved in DMSO and added to the cells in 50 μl HBSS, yielding a final DMSO concentration of 0.2%. Fluorescence was monitored for 10 minutes at 1 second intervals at 470-495 nm/515-575 nm excitation/emission wavelengths. Changes of membrane potential were measured using a similar procedure, except that the cells were loaded for 2 hours with a proprietary membrane potential dye (Molecular Devices, Cat # R8042) dissolved in HBSS. The dye loading solution was discarded and replaced with 50 ul of 20 mM HEPES, 11.1 mM Glucose, 125 mM NaCl, and 2.5 mM KCl, and 20 mM CaCl₂. The test compounds dissolved in DMSO were added to the cells in 50 μl of the same solution, yielding a final DMSO concentration of 0.2%. Fluorescence was monitored for 10 minutes at 1 second intervals at 510-545 nm/565-625 nm excitation/emission.

EC₅₀ values were calculated using GraphPad Prism (GraphPad Software).

Two-Electrode Voltage Clamping of Xenopus laevis Oocytes.

Oocytes were placed in a RC-8 recording chamber (Warner Instruments) filled with ND-96 bath solution (in mM: NaCl 96, KCl 2, MgCl2 1, CaCl2 1.8, and HEPES 5, pH 7.5,). Glass microelectrodes filled with 3 M KCl and with resistances between 0.9-1.4 MΩ were used to voltage clamp the oocytes through a Turbo Tec 10-C amplifier (npi) and ITC-16 computer interface (InstruTech) controlled by PatchMaster recording software v.2x73.5 (HEKA). Oocytes were perfused with ND-96 saline at 2 mL/min (gravity fed). N-deacetylatedpyrifluquinazon (dPFQ) was delivered through the same perfusion line directly over the cell by switching out the bath solution with 10 uM dPFQ diluted in ND-96 and 0.1% DMSO. Between voltage steps, oocytes were held at −20 mV. Every 10 seconds, the voltage was stepped to +40 mV and the current, after equilibration, was measured and plotted over time as chart recordings (FIG. 4a). Current voltage relationships for single and co-expressed subunits were determined by measuring currents elicited by stepping the voltage from the holding potential of −20 mV to voltages between −100 through +60 mV. Chart recordings and IV tests were exported from Patchmaster and analyzed and plotted with Prism 6 (GraphPad). Chart recording currents were normalized to the initial current for each cell (I/I₀). N-deacetylated pyrifluquinazon evoked both inward and outward current responses only in oocytes expressing both Nan and Iav (FIG. 4a), but neither current responses inward or outward were evoked in oocytes expressing Nan or Iav alone. Accordingly, a method for determining whether or not a candidate compound may be a modulator of an insect TRPV channel may include providing a first cell expressing an insect TRPV channel, wherein that first cell may be a Xenopus laevis oocyte; contacting the first cell with a candidate compound, wherein the candidate compound may be dPFQ; and assaying for modulation of the insect TRPV channel, wherein the modulation identifies the candidate compound as the modulator of the insect TRPV channel and further wherein the assaying step comprises at least of one the following steps: comparing the electrical current between the first cell in the absence of the candidate compound with the electrical current in the first cell in the presence of the candidate compound; and/or comparing the electrical current between the first cell in the presence of the candidate compound and having one or more Nanchung proteins and one or more Inactive proteins with the electrical current of the first cell in the presence of the candidate compound and not having one or more Nanchung proteins and/or one or more Inactive proteins.

Membrane Preparation

CHO-K1 cells were transduced with adenoviruses expressing AcGFP-tagged Nan and Iav either alone, or as 1:1 mixture (4000 viral particles per cell). The adenovirus-transduced cells were seeded at 10×10⁶ cells per T175 flask (Greiner) and kept at 37° C. 18 hrs followed by incubation at 25° C. for 72 hrs. The media was changed at 24 hrs and 72 hrs post transduction. The cells were rinsed twice with PBS, and incubated in Versene solution (Life Technologies) for 10 min at room temperature. Detached cells were pelleted at 300×g for 10 min and resuspended in 8 mL of ice-cold 20 mM Hepes (pH 7.3) containing 1% (wt/vol) protease inhibitors mixture (Halt; Thermo Fisher). After 10 min on ice, the cells were homogenized by sonication. The samples were centrifuged at 300×g for 10 min, and supernatants were collected and centrifuged for 2 h at 50,000×g. Membranes were resuspended in 0.5 mL of 50 mM HEPES, 100 mM NaCl, 1 mM CaCl₂, 5 mM MgCl₂, pH 7.4, and stored at −80° C. Protein content was determined using the Bradford Method.

[³H]-Afidopyropen Binding Assay

Equilibrium binding assays were performed in the binding buffer (50 mM HEPES, 100 mM NaCl, 1 mM CaCl₂, 5 mM MgCl₂, pH 7.4). The reactions were performed in 96-well U-bottom polypropylene plates. 100 μL membranes resuspended in the binding buffer containing Tween-20 (0.0025% final concentration in the assay), were combined with either 200 μl (saturation binding experiments), or 800 μl (ligand displacement experiments) of binding buffer containing [³H]-afidopyropen, dissolved in ethanol, and, where indicated, test compounds, dissolved in DMSO. The final concentrations of ethanol and DMSO were kept at 0.2% and 0.25% respectfully in both saturation binding and competition experiments. Typically, 60-100 μg of membranes were used per data point. Followed by 3 hours of incubation at room temperature, assay mixes were filtered through Multiscreen HTS+96 Hi Flow FC filters (EMD Millipore), pre-wetted with 0.1% polyethyleneimine. Filters were washed three times with 200 μl of ice-cold binding buffer, scintillation cocktail was added and radioactivity was measured using Wallac 1450 Microbeta counter. Non-specific binding was measured in the presence of 500-fold excess of unlabeled Afidopyropen. Typically, ligand depletion accounted for less than 10% of total ligand. In saturation binding studies, at the lowest concentrations of [³H]-afidopyropen, equal to 49 pM and 97.7 pM, ligand depletion was 19% and 14%, respectfully. That was corrected by assuming that concentration of free ligand was equal to total minus bound concentration. Nonlinear regression analysis was performed using GraphPad Prism (GraphPad Software), and Kᵢ values were calculated by Cheng-Prusoff method (Cheng and Prusoff, 1973).

It is therefore intended that the foregoing detailed description be regarded as illustrative rather than limiting, and that it be understood that it is the following claims, including all equivalents, that are intended to define the spirit and scope of this invention.

### REFERENCES

Ausborn, J., Wolf, H., Mader, W., and Kayser, H. (2005). The insecticide pymetrozine selectively affects chordotonal mechanoreceptors. The Journal of experimental biology 208, 4451-4466.
Casida, J. E., and Durkin, K. A. (2013). Neuroactive insecticides: targets, selectivity, resistance, and secondary effects. Annual review of entomology 58, 99-117.
Cheng, Y., and Prusoff, W. H. (1973). Relationship between the inhibition constant (K1) and the concentration of inhibitor which causes 50 percent inhibition (I50) of an enzymatic reaction. Biochemical pharmacology 22, 3099-3108.
Kamikouchi, A., Inagaki, H. K., Effertz, T., Hendrich, O., Fiala, A., Gopfert, M. C., and Ito, K. (2009). The neural basis of Drosophila gravity-sensing and hearing. Nature 458, 165-171.
Kavlie, R. G., and Albert, J. T. (2013). Chordotonal organs. Current biology: CB 23, R334-335.
Leichter, C. A., Thompson, A., Johnson, B. R., and Scott, J. G. (2013). The high potency of ME-5343 to aphids is due to a unique mechanism of action. Pesticide Biochemistry and Physiology, 169-175.
Maienfisch, P. (2012). Selective feeding blockers: pymetrozine, flonicamid, and pyrifluquinanzon. In Modern Crop Protection Compounds W. Kramer, U. Schirmer, P. Jenschke, and M. Witschel, eds. (New York: John Wiley and Sons), pp. 1327-1346.
Marshall, I. C., Owen, D. E., and McNulty, S. (2013). Measuring Ca(2)(+) changes in multiwell format using the Fluorometric Imaging Plate Reader (FLIPR®). Methods in molecular biology 937, 103-109.
Nesterov, A., Kandasamy, R., Wedel, B., and Salgado, V. L. (2013). Methods for determining modulators of insect transient receptor potential V (TRPV) channel.
Nesterov, A., Spalthoff, C., Kandasamy, R., Katana, R., Rankl, N. R., Andrés, M., Jade, P., Dorsch, J. A., Stam, L. F., Braun, F., et al. (2015). TRP channels in insect stretch receptors as insecticide targets. Neuron 86, 665-671.
Postle, K., Nguyen, T. T., and Bertrand, K. P. (1984). Nucleotide sequence of the repressor gene of the TN10 tetracycline resistance determinant. Nucleic acids research 12, 4849-4863.
Sun, Y., Liu, L., Ben-Shahar, Y., Jacobs, J. S., Eberl, D. F., and Welsh, M. J. (2009). TRPA channels distinguish gravity sensing from hearing in Johnston's organ. Proceedings of the National Academy of Sciences of the United States of America 106, 13606-13611.
Thorneloe, K. S., Sulpizio, A. C., Lin, Z., Figueroa, D. J., Clouse, A. K., McCafferty, G. P., Chendrimada, T. P., Lashinger, E. S., Gordon, E., Evans, L., et al. (2008). N-((1S)-1-{[4-((2S)-2-{[(2,4-dichlorophenyl)sulfonyl]amino}-3-hydroxypropanoyl)-1-piperazinyl]carbonyl}-3-methylbutyl)-1-benzothiophene-2-carboxamide (GSK1016790A), a novel and potent transient receptor potential vanilloid 4 channel agonist induces urinary bladder contraction and hyperactivity: Part I. The Journal of pharmacology and experimental therapeutics (2008) August: 326, 432-442.
Venkatachalam, K., and Montell, C. (2007). TRP channels. Annual review of biochemistry 76, 387-417.
Zheng, W., Spencer, R. H., and Kiss, L. (2004). High throughput assay technologies for ion channel drug discovery. Assay and drug development technologies 2, 543-552.
Kim J, Chung Y D, Park D Y, Choi S, Shin D W, Soh H, Lee H W, Son W, Yim J, Park C S, Kernan M J, Kim C. A TRPV family ion channel required for hearing in Drosophila. Nature. 2003 Jul. 3; 424(6944):81-4. Epub 2003 Jun. 18. PubMed PMID: 12819662.
Gong Z, Son W, Chung Y D, Kim J, Shin D W, McClung C A, Lee Y, Lee H W, Chang D J, Kaang B K, Cho H, Oh U, Hirsh J, Kernan M J, Kim C. Two interdependent TRPV channel subunits, inactive and Nanchung, mediate hearing in Drosophila. J Neurosci. 2004 Oct. 13; 24(41):9059-66. PubMed PMID: 15483124.
Chang G C, Snyder W E. Pymetrozine causes a nontarget pest, the Colorado potato beetle (Coleoptera: Chrysomelidae), to leave potato plants. J Econ Entomol. 2008 February; 101(1):74-80. PubMed PMID: 18330119.
Douglas J T. Adenovirus-mediated gene delivery: an overview. Methods Mol Biol. 2004; V246:3-14. Review. PubMed PMID: 14970581.

1-38: (canceled)

39. A method for determining whether a compound binds directly to one or more Nanchung proteins, comprising:
(a) providing one or more Nanchung proteins;
(b) contacting the one or more Nanchung, proteins with one or more compounds; and
(c) determining the binding affinity of the one or more compounds to the one or more Nanchung proteins.

40. The method of claim 39, further comprising providing one or more TRP proteins and contacting said Nanchung proteins therewith, wherein the one or more TRP proteins form a complex with the one or more Nanchung proteins.

41. The method of claim 40, wherein the one or more TRP proteins are one or more TRPV proteins.

42. The method of claim 41, wherein the one or more TRPV proteins is an Inactive protein.

43. The method of claim 39, wherein the one or more Nanchung proteins form one or more TRP and/or TRPV channels.

44. The method of claim 39, wherein the one or more compounds includes at least one labeled compound.

45. The method of claim 39, wherein the one or more compounds includes at least one labeled compound and at least one candidate compound.

46. A method for classifying a compound, comprising:
(1) performing a binding assay step, wherein one or more Nanchung proteins are contacted by one or more candidate compounds and determining the binding affinity for the one or more candidate compounds to the one or more Nanchung proteins,
(2) identifying a mode of action classification for the insecticide compound comprising the compound.

47. The method of claim 46, wherein the method for classifying a candidate compound further comprises:
performing a modulator assay step, wherein one or more TRP and/or TRPV channels are contacted with one or more candidate compounds and an assay is used to determine whether the one or more candidate compounds acts as a modulator of the one or more TRP and/or TRPV channels.

48. The method of claim 46, wherein the binding assay step comprises one or more of the following steps:
(a) providing one or more of the following: Nanchung proteins, Inactive proteins, TRP and/or TRPV proteins, protein complexes and/or channels;
(b) contacting the one or more Nanchung proteins, TRP, and/or TRPV protein complexes and/or channels with one or more compounds;
(c) measuring the amount of the one or more compounds which are bound to the one or more Nanchung proteins, TRP, and/or TRPV protein complexes and/or channels;
(d) calculating the equilibrium disassociation constant (K_{d}) for the one or more compounds, wherein the K_{d} may be determined using one or more of the following methods: (I)(a) maximum binding (Bₘₐₓ) and (b) the concentration of compound needed to reach 50% of maximum binding, (II) measuring association (Kon) and dissociation (Koff) rates of the compound where Kd=Koff/Kon, (III) fluorescence polarization assay, (IV) surface plasmon resonance, (V) isothermal calorimetry, and (VI) Schild analysis,
(e) contacting the one or more Nanchung proteins, TRP, and/or TRPV protein complexes and/or channels with a labeled compound having high affinity to the one or more Nanchung proteins, TRP, and/or TRPV protein complexes and/or channels;
(f) contacting the one or more one or more Nanchung proteins, TRP, and/or TRPV protein complexes and/or channels with at least one candidate compound in the presence of the one or more labeled compounds;
(g) increasing the amount of the at least one candidate compound available to contact the one or more Nanchung proteins, TRP, and/or TRPV protein complexes and/or channels until binding of the labeled compound is near 0%;
(h) calculating the equilibrium inhibition constant (Kᵢ) and/or the equilibrium dissociation constant (K_{d}) for competition assay using one or more of the following methods: (I) (a) determining amount of bound labeled compound in the absence of the candidate compound and (b) determining concentration of the candidate compound that is required to reduce the amount of bound labeled compound by 50%, (II) fluorescent polarization assay, and/or (III) Schild analysis;
(i) comparing the inhibition constant of the candidate compounds to other candidate compounds inhibition constants;
(j) comparing the inhibition constant of the candidate compounds to the disassociation consonant of the labeled compound; and
(k) identifying one or more candidate compounds having a high affinity to the one or more Nanchung proteins, Inactive proteins, TRP and/or TRPV proteins, protein complexes and/or channels.

49. The method of claim 46, wherein the candidate compound is selected from the group consisting of small organic molecules, small inorganic molecules, polysaccharides, peptides, proteins, nucleic acids, extracts made from biological materials, and any combination thereof.

50. A composition comprising one or more compounds that directly bind to one or more insect TRP and/or TRPV protein complexes.

51. A composition comprising one or more compounds selected by the method of claim 39.

52. A composition comprising one or more compounds selected by the method of claim 46.

53. A method of insect control comprising applying the composition of claim 50 to one or more of: an insect, an insect habitat, a bait, a trap and/or an insect feeding source.

54. A method of insect control comprising applying the composition of claim 51 to one or more of: an insect, an insect habitat, a bait, a trap and/or an insect feeding source.

55. A method of insect control comprising applying the composition of claim 52 to one or more of: an insect, an insect habitat, a bait, a trap and/or an insect feeding source.

56. The method of claim 53, wherein the compound is an inhibitor of an insect TRP channel.

57. The method of claim 53, wherein the compound is an activator of an insect TRP channel.

58. A method for determining whether a compound binds directly to the same site on one or more Nanchung proteins, comprising:
(a) providing one or more Nanchung proteins;
(b) contacting the one or more Nanchung proteins with one or more compounds, wherein the one or more compounds is selected from the group consisting of: afidopyropen, pymetrozine, pyrifluquinazon and/or N-deacetylated-pyrifluquinazon;
(c) contacting the one or more Nanchung proteins with one or more candidate compounds; and
(d) determining the binding affinity of the one or more candidate compounds to the one or more Nanchung proteins.
